(19) United States
(12) Patent Application Publication
(10) Pub. No.: US 2014/0273163 A1
(43) Pub. Date: Sep. 18, 2014

(54) OLIGOSACCHARIDE COMPOSITIONS, GLYCOPROTEINS AND METHODS TO PRODUCE THE SAME IN PROKARYOTES

(71) Applicant: Glycobia, Inc., Ithaca, NY (US)

(72) Inventors: Adam C Fisher, Ithaca, NY (US); Judith H Merritt, Ithaca, NY (US); Brian S Hamilton, Ithaca, NY (US); Juan D Valderrama-Rincon, Bogota (CO); Matthew P DeLisa, Ithaca, NY (US)

(73) Assignee: Glycobia, Inc., Ithaca, NY (US)

(21) Appl. No.: 14/210,559

(22) Filed: Mar. 14, 2014

Related U.S. Application Data
(60) Provisional application No. 61/785,586, filed on Mar. 14, 2013.

(51) Int. Cl. C12N 15/70

(52) U.S. Cl. CPC C12N 15/70
USPC 435/252.33

(57) ABSTRACT

Disclosed are methods and compositions to produce various oligosaccharide compositions and glycoproteins. Prokaryotic hosts cells are cultured under conditions effective to produce human-like e.g., high-mannose, hybrid and complex glycosylation patterns by introducing glycosylation pathways into the host cells.

### STATEMENT OF FEDERALLY SPONSORED RESEARCH AND DEVELOPMENT

This invention was made with government support under grant numbers 1R43GM088905-01, 2R44GM088905-02 and 5R44GM088905-03 by the National Institutes of Health. The government has certain rights in this invention.

### SEQUENCE LISTING

The instant application contains a Sequence Listing which has been submitted electronically in ASCII format and is hereby incorporated by reference in its entirety. Said ASCII copy, created on Mar. 6, 2014, is named GLY-103 SL.txt and is 94,020 bytes in size.

### FIELD OF INVENTION

The present invention generally relates to the field of glycobiology and protein engineering. More specifically, the embodiments described herein relates to oligosaccharide compositions and therapeutic glycoprotein production in prokaryotes.

### BACKGROUND OF THE INVENTION

### Glycotherapeutics

Protein-based therapeutics currently represent one in every four new drugs approved by the FDA (Walsh, G., “Biopharmaceutical Benchmarks,” Nat Biotechnol 18:831-3 (2000); Walsh, G, “Biopharmaceutical Benchmarks,” Nat Biotechnol 21:865-70 (2003); and Walsh, G, “Biopharmaceutical Benchmarks,” Nat Biotechnol 24:769-76 (2006)).

While several protein therapeutics can be produced using a prokaryotic expression system such as E. coli (e.g., insulin), the vast majority of therapeutic proteins require additional post-translational modifications, thought to be absent in prokaryotes, to attain their full biological function. In particular, N-linked protein glycosylation is predicted to affect more than half of all eukaryotic protein species (Apweiler et al., “On the Frequency of Protein Glycosylation, as Deduced From Analysis of the SWISS-PROT Database,” Biochim Biophys Acta 1473:4-8 (1999)) and is often essential for proper folding, pharmacokinetic stability, tissue targeting and efficacy for a large number of proteins (Helenius et al., “Intracellular Functions of N-linked Glycans,” Science 291:2364-9 (2001)). Since most bacteria do not glycosylate their own proteins, expression of most therapeutically relevant glycoproteins, including antibodies, is relegated to mammalian cells. However, mammalian cell culture suffers from a number of drawbacks including: (i) extremely high manufacturing costs and low volumetric productivity of eukaryotic hosts, such as CHO cells, relative to bacteria; (ii) retroviral contamination; (iii) the relatively long time required to generate stable cell lines; (iv) relative inability to rapidly generate stable, “high-producing” eukaryotic cell lines via genetic modification; and (v) high product variability created by glycoform heterogeneity that arises when using host cells, such as CHO, that have endogenous non-human glycosylation pathways (Choi et al., “Use of Combinatorial Genetic Libraries to Humanize N-linked Glycosylation in the Yeast Pichia pastoris,” Proc Natl Acad Sci USA 100:5022-7 (2003)). Expression in E. coli, on the other hand, does not suffer from these limitations.

Expression of Therapeutic Proteins in E. coli

Many therapeutic recombinant proteins are currently expressed using E. coli as a host organism. One of the best examples is human insulin, which was first produced in E. coli by Eli Lilly in 1982. Since that time, a vast number of human therapeutic proteins have been approved in the U.S. and Europe that rely on E. coli expression, including human growth hormone (hGH), granulocyte macrophage colony stimulating factor (GM-CSF), insulin-like growth factor (IGF-1, IGFBP-3), keratinocyte growth factor, interferons (IFN-α, IFN-β1b, IFN-γ1b), interleukins (IL-1, IL-2, IL-11), tissue necrosis factor (TNF-α), and tissue plasminogen activator (tPA). However, almost all glycoproteins are produced in mammalian cells. When a protein that is normally glycosylated is expressed in E. coli, the lack of glycosylation in that host can yield proteins with impaired function. For instance, aglycosylated human monoclonal antibodies (mAbs) (e.g., anti-tissue factor IgG1) can be expressed in soluble form and at high levels in E. coli (Simmons et al., “Expression of Full-length Immunoglobulins in Escherichia coli: Rapid and Efficient Production of Aglycosylated Antibodies,” J Immunol Methods 263:133-47 (2002)). However, while E. coli-derived mAbs retained tight binding to their cognate antigen and neonatal receptor and exhibited a circulating half-life comparable to mammalian cell-derived antibodies, they were incapable of binding to C1q and the FcγRI receptor due to the absence of N-glycan.

### Eukaryotic and Prokaryotic N-Linked Protein Glycosylation

N-linked protein glycosylation is an essential and conserved process occurring in the endoplasmic reticulum (ER) of eukaryotic organisms (Burda et al., “The Dolichol Pathway of N-linked Glycosylation,” Biochim Biophys Acta 1426:239-57 (1999)). It is important for protein folding, oligomerization, quality control, sorting, and transport of secretory and membrane proteins (Helenius et al., “Intracellular Functions of N-linked Glycans,” Science 291:2364-9 (2001)). The eukaryotic N-linked protein glycosylation pathway can be divided into two different processes: (i) the assembly of the lipid-linked oligosaccharide at the membrane of the endoplasmic reticulum and (ii) the transfer of the oligosaccharide from the lipid anchor dolichol pyrophosphate to selected asparagine residues of nascent polypeptides. The characteristics of N-linked protein glycosylation, namely (i) the use of dolichol pyrophosphate (Dol-PP) as carrier for oligosaccharide assembly, (ii) the transfer of only the completely assembled Glc₃Man₉GlcNAc₂ oligosaccharide, and (iii) the recognition of asparagine residues characterized by the sequence N-X-S/T where N is asparagine, X is any amino acid except proline, and S/T is serine/threonine (Gavel et al., “Sequence Differences Between Glycosylated and Non-glycosylated Asn-X-Thr/Ser Acceptor Sites: Implications for Protein Engineering,” Protein Eng 3:433-42 (1990)) are highly conserved in eukaryotes. The oligosaccharyltransferase (OST) catalyzes the transfer of the oligosaccharide from the lipid donor dolichylpyrophosphate to the acceptor protein. In yeast, eight different membrane proteins have been identified that constitute the complex in vivo (Kelleher et al., “An Evolving View of the Eukaryotic Oligosaccharyltransferase,” Glycobiology 16:47R-62R (2006)). STT3 is thought to represent the catalytic subunit of the OST (Nilsson et al., “Photocross-linking of Nascent Chains to the STT3 Subunit of the Oligosaccharyltransferase Complex,” J Cell Biol 161:715-25 (2003) and Yan et al., “Studies on the Function of Oligosaccharyl Transferase Subunits. Stt3p is Directly Involved in the Glycosylation Process,” J Biol Chem 277:47692-700 (2002)). It is the most conserved subunit in the OST complex (Burda et al., “The Dolichol Pathway of N-linked Glycosylation,” Biochim Biophys Acta 1426:239-57 (1999)).

Conversely, the lack of glycosylation pathways in bacteria has greatly restricted the utility of prokaryotic expression hosts for making therapeutic proteins, especially since by certain estimates “more than half of all proteins in nature will eventually be found to be glycoproteins” (Apweiler et al., “On the Frequency of Protein Glycosylation, as Deduced From Analysis of the SWISS-PROT Database,” Biochim Biophys Acta 1473:4-8 (1999)). Recently, however, it was discovered that the genome of a pathogenic bacterium, C. jejuni, encodes a pathway for N-linked protein glycosylation (Szymanski et al., “Protein Glycosylation in Bacterial Mucosal Pathogens,” Nat Rev Microbiol 3:225-37 (2005)). The genes for this pathway, first identified in 1999 by Szymanski and coworkers (Szymanski et al., “Evidence for a System of General Protein Glycosylation in Campylobacter jejuni,” Mol Microbiol 32:1022-30 (1999)), comprise a 17-kb locus named pgl for protein glycosylation. Following discovery of the pgl locus, in 2002 Linton et al. identified two C. jejuni glycoproteins, PEB3 and CgpA, and showed that C. jejuni-derived glycoproteins such as these bind to the N-acetyl galactosamine (GalNAc)-specific lectin soybean agglutinin (SBA) (Linton et al., “Identification of N-acetylgalactosamine-containing Glycoproteins PEB3 and CgpA in Campylobacter jejuni,” Mol Microbiol 43:497-508 (2002)). Shortly thereafter, Young et al. identified more than 30 potential C. jejuni glycoproteins, including PEB3 and CgbA, and used mass spectrometry and NMR to reveal that the N-linked glycan was a heptasaccharide with the structure GalNAc-α1,4-GalNAc-α1,4-[Glcβ1,3]GalNAc-α1,4-GalNAc-α1,4-GalNAc-α1,3-Bac-β1,N-Asn (GalNAc₅GlcBac, where Bac is bacillosamine or 2,4-diacetamido-2,4,6-trideoxyglucose) (Young et al., “Structure of the N-linked Glycan Present on Multiple Glycoproteins in the Gram-negative Bacterium, Campylobacter jejuni,” J Biol Chem 277:42530-9 (2002)). The branched heptasaccharide is synthesized by sequential addition of nucleotide-activated sugars on a lipid carrier undecaprenylpyrophosphate (Und-PP) on the cytoplasmic side of the inner membrane (Feldman et al., “Engineering N-linked Protein Glycosylation with Diverse O Antigen Lipopolysaccharide Structures in Escherichia coli,” Proc Natl Acad Sci USA 102:3016-21 (2005)) and, once assembled, is flipped across the membrane by the putative ATP-binding cassette (ABC) transporter WlaB (Alaimo et al., “Two Distinct But Interchangeable Mechanisms for Flipping of Lipid-linked Oligosaccharides,” Embo J 25:967-76 (2006) and Kelly et al., “Biosynthesis of the N-linked Glycan in Campylobacter jejuni and Addition Onto Protein Through Block Transfer,” J Bacteriol 188:2427-34 (2006)). Next, transfer of the heptasaccharide to substrate proteins in the periplasm is catalyzed by an OST named PglB, a single, integral membrane protein with significant sequence similarity to the catalytic subunit of the eukaryotic OST STT3 (Young et al., “Structure of the N-linked Glycan Present on Multiple Glycoproteins in the Gram-negative Bacterium, Campylobacter jejuni,” J Biol Chem 277:42530-9 (2002)). PglB attaches the heptasaccharide to asparagine in the motif D/E-X₁-N-X₂-S/T (where D/E is aspartic acid/glutamic acid, X₁ and X₂ are any amino acids except proline, N is asparagine, and S/T is serine/threonine), a sequon similar to that used in the eukaryotic glycosylation process (N-X-S/T) (Kowarik et al., “Definition of the Bacterial N-glycosylation Site Consensus Sequence,” Embo J 25:1957-66 (2006)).

### Glycoengineering of Microorganisms

A major problem encountered when expressing therapeutic glycoproteins in mammalian, yeast, or even bacterial host cells is the addition of non-human glycans. For instance, yeast, one of the two most frequently used systems for the production of therapeutic glycoproteins, transfer highly immunogenic mannan-type N-glycans (containing up to one hundred mannose residues) to recombinant glycoproteins. Mammalian expression systems can also modify therapeutic proteins with non-human sugar residues, such as the N-glycosylneuraminic acid (Neu5Gc) form of sialic acid (produced in CHO cells and in milk) or the terminal α(1,3)-galactose (Gal) (produced in murine cells). Repeated administration of therapeutic proteins carrying non-human sugars can elicit adverse reactions, including an immune response in humans.

As an alternative to using native glycosylation systems for producing therapeutic glycoproteins, the availability of glyco-engineered expression systems could open the door to customizing the glycosylation of a therapeutic protein and could lead to the development of improved therapeutic glycoproteins. Such a system would have the potential to eliminate undesirable glycans and perform human glycosylation to a high degree of homogeneity. The yeast Pichia pastoris has been glyco-engineered to provide an expression system with the capacity for glycosylation for specific therapeutic functions (Gerngross, T. U., “Advances in the Production of Human Therapeutic Proteins in Yeasts and Filamentous fungi,” Nat Biotechnol 22:1409-14 (2004); Hamilton et al., “Glycosylation Engineering in Yeast: The Advent of Fully Humanized Yeast,” Curr Opin Biotechnol 18:387-92 (2007); and Wildt et al., “The Humanization of N-glycosylation Pathways in Yeast,” Nat Rev Microbiol 3:119-28 (2005)).

For example, a panel of glyco-engineered P. pastoris strains was used to produce various glycoforms of the monoclonal antibody Rituxan (an anti-CD20IgG1 antibody) (Li et al., “Optimization of Humanized IgGs in Glycoengineered Pichia pastoris,” Nat Biotechnol 24:210-5 (2006)). Although these antibodies share identical amino acid sequences to commercial Rituxan, specific glycoforms displayed ˜100-fold higher binding affinity to relevant FcγRIII receptors and exhibited improved in vitro human B-cell depletion (Li et al., “Optimization of Humanized IgGs in Glycoengineered Pichia pastoris,” Nat Biotechnol 24:210-5 (2006)). The tremendous success and potential of glyco-engineered P. pastoris is not without some drawbacks. For instance, in yeast and all other eukaryotes N-linked glycosylation is essential for viability (Herscovics et al., “Glycoprotein Biosynthesis in Yeast,” FASEB J 7:540-50 (1993) and Zufferey et al., “STT3, a Highly Conserved Protein Required for Yeast Oligosaccharyl Transferase Activity In Vivo,” EMBO J 14:4949-60 (1995)). Gerngross and coworkers systematically eliminated and re-engineered many of the unwanted yeast N-glycosylation reactions (Choi et al., “Use of Combinatorial Genetic Libraries to Humanize N-linked Glycosylation in the Yeast Pichia pastoris,” Proc Natl Acad Sci USA 100:5022-7 (2003)). However, elimination of the mannan-type N-glycans is only half of the glycosylation story in yeast. This is because yeast also perform O-linked glycosylation whereby O-glycans are linked to Ser or Thr residues in glycoproteins (Gentzsch et al., “The PMT Gene Family: Protein O-glycosylation in Saccharomyces cerevisiae is Vital,” EMBO J 15:5752-9 (1996)). As with N-linked glycosylation, O-glycosylation is essential for viability (Gentzsch et al., “The PMT Gene Family: Protein O-glycosylation in Saccharomyces cerevisiae is Vital,” EMBO J 15:5752-9 (1996)) and thus cannot be genetically deleted from glyco-engineered yeast. Since there are differences between the O-glycosylation machinery of yeast and humans, the possible addition of O-glycans by glyco-engineered yeast strains has the potential to provoke adverse reactions including an immune response.

Aebi and his coworkers transferred the C. jejuni glycosylation locus into E. coli and conferred upon these cells the extraordinary ability to post-translationally modify proteins with N-glycans (Wacker et al., “N-linked Glycosylation in Campylobacter jejuni and its Functional Transfer into E. coli,” Science 298:1790-3 (2002)). However, despite the functional similarity shared by the prokaryotic and eukaryotic glycosylation mechanisms, the oligosaccharide chain attached by the prokaryotic glycosylation machinery (GalNAc₅GlcBac) is structurally distinct from that attached by eukaryotic glycosylation pathways (Szymanski et al., “Protein Glycosylation in Bacterial Mucosal Pathogens,” Nat Rev Microbiol 3:225-37 (2005); Young et al., “Structure of the N-linked Glycan Present on Multiple Glycoproteins in the Gram-negative Bacterium, Campylobacter jejuni,” J Biol Chem 277:42530-9 (2002); and Weerapana et al., “Asparagine-linked Protein Glycosylation: From Eukaryotic to Prokaryotic Systems,” Glycobiology 16:91R-101R (2006)). Numerous attempts (without success) have been made to reprogram E. coli with a eukaryotic N-glycosylation pathway to express N-linked glycoproteins with structurally homogeneous human-like glycans.

More recently, Vaderrama-Rincon et al. “An engineered eukaryotic protein glycosylation pathway in E. coli,” Nat Chem Bio 8, 434-436 (2012) showed that prokaryotic host cells can be glycoengineered with eukaryotic glycosyltransferases. Specifically, expression of UDP-GlcNAc transferases and GDP-mannose transferases in a prokaryotic host cell demonstrated the production of the trimannosyl core structure, which is the basis of nearly all eukaryotic N-linked oligosaccharide structures. Fully elaborated human-like glycans, however, still require additional glycol-engineering.

What is needed, therefore, is a method to produce human-like glycans such as high-mannose, hybrid and complex types.

### SUMMARY OF THE INVENTION

The invention provides methods and materials for the production of oligosaccharide compositions and for the production of recombinant glycoproteins in prokaryotic host cells. Various glycoprotein compositions comprising specific N-glycans are produced using the methods of the invention. In certain embodiments, desired glycoforms are produced as the predominant species.

The invention also provides methods and materials for the production of vaccines antigens comprising specific oligosaccharide compositions, for example, to induce immunity or immunological tolerance (e.g., anergy) within a subject. Various aspects of the present invention are directed to antigen-carbohydrate conjugates able to bind lectins expressable on the surfaces of dendritic cell and/or other antigen-presenting cell.

A first aspect of the invention relates to a method of producing an oligosaccharide composition, said method comprising: culturing a recombinant prokaryotic host cell that produces an oligosaccharide composition having a terminal mannose residue to express one or more N-acetylglucosaminyl transferase enzyme activity (EC 2.4.1.101; EC 2.4.1.143; EC 2.4.1.145; EC 2.4.1.155; EC 2.4.1.201) that catalyzes the transfer of a UDP-GlcNAc residue onto said terminal mannose residue, said culturing step carried out under conditions effective to produce an oligosaccharide composition having a terminal GlcNAc residue.

A second aspect of the invention relates to a method of producing an oligosaccharide composition, said method comprising: culturing a host cell to express one or more galactosyltransferase enzyme activity (EC 2.4.1.38) that catalyzes the transfer of a UDP-Galactose residue onto said terminal GlcNAc residue, said culturing step carried out under conditions effective to produce an oligosaccharide composition having a terminal galactose residue.

A third aspect of the invention relates to a method of producing an oligosaccharide composition, said method comprising: culturing the host cell to express one or more sialyltransferase enzyme activity (EC 2.4.99.4 and EC 2.4.99.1) that catalyzes the transfer of a CMP-NANA residue onto said terminal galactose residue, said culturing step carried out under conditions effective to produce an oligosaccharide composition having a terminal sialic acid residue.

Other aspects of the invention relate to expression of one or more of the enzymes as solubility enhanced fusion proteins. Further aspects of the invention include transfer of the glycans onto a gene encoding a protein of interest, whereby the host cell produces a glycosylated protein.

Additional aspects include culturing conditions and overexpression of additional enzymes for the production of predominant glycoforms. Featured aspects of the invention provide prokaryotic host cells to express various glycosyltransferase activities to produce high-mannose, hybrid and/or complex oligosaccharide compositions as well as high-mannose, hybrid and/or complex glycosylated proteins.

Generally, the present invention commercializes technologies for the design, discovery, and development of glycoprotein therapeutics and diagnostics. Specifically, the present invention provides for the development of an efficient, low-cost strategy for efficient production of authentic human glycoproteins in microbial cells. In various aspects, the glyco-engineered bacteria of the invention are capable of stereospecific production of N-linked glycoproteins. In one embodiment, bacteria are transformed with genes encoding a novel glycosylation pathway that is capable of efficiently glycosylating target proteins at specific asparagine acceptor sites (e.g., N-linked glycosylation). Using these specially engineered cell lines, various recombinant protein-of-interest can be expressed and glycosylated.

Further, the invention provides methods for engineering permutations of oligosaccharide structures in prokaryotes, which is expected to alter e.g., pharmacokinetic properties of proteins and elucidate the role of glycosylation in biological phenomena. The invention, therefore, provides biotechnological synthesis of therapeutic proteins, novel glycoconjugates, immunostimulating agents (e.g., vaccines) for research, industrial, and therapeutic applications.

### BRIEF DESCRIPTION OF THE DRAWINGS

FIG. 1. Production of a high-mannose type Man₅GlcNAc₂ glycoform. MALDI-TOF mass spectra of lipid-released glycans (A) extracted from GLY02 consistent with the expected Man₅GlcNAc₂ (m/z 1257.6) glycoform and (B) further treated with an α1,2-mannosidase consistent with the expected Man₃GlcNAc₂ glycoform (m/z 933.4).

FIG. 2. Production of a hybrid GlcNAcMan₃GlcNAc₂ glycoform. MALDI-TOF mass spectra of lipid-released glycans (A) extracted from GLY03 consistent with the expected GlcNAcMan₃GlcNAc₂ glycoform (m/z 1136.5) and (B) further treated with a β-N-acetylglucosaminidase consistent with the expected Man₃GlcNAc₂ glycoform (m/z 933.5).

FIG. 3. Production of a complex GlcNAc₂Man₃GlcNAc₂ glycoform. MALDI-TOF mass spectrum of lipid-released glycans extracted from GLY06.1 consistent with the expected GlcNAc₂Man₃GlcNAc₂ glycoform (m/z 1339.8).

FIG. 4. Production of a hybrid, branched glycoform. MALDI-TOF mass spectra of lipid-released glycans (A) extracted from GLY05 consistent with the expected GlcNAc₂Man₃GlcNAc₂ glycoform (m/z 1339.7) and (B) further treated with a β-N-acetylglucosaminidase consistent with the expected Man₃GlcNAc₂ glycoform (m/z 933.5).

FIG. 5. Production of a multiple-antennary glycoform. MALDI-TOF mass spectrum of (A) glycans synthesized ex vivo and (B) lipid-released glycans extracted from GLY06.4 consistent with the expected GlcNAc₃Man₃GlcNAc₂ (m/z 1543.1).

FIG. 6. Production of a GalGlcNAcMan₃GlcNAc₂ glycoform. MALDI-TOF mass spectrum of lipid-released glycans extracted from GLY04.1 consistent with the expected GalGlcNAcMan₃GlcNAc₂ glycoform (m/z 1298.7).

FIG. 7. Production of a Gal₂GlcNAc₂Man₃GlcNAc₂ glycoform. MALDI-TOF mass spectrum of (A) glycans synthesized ex vivo and (B) lipid-released glycans extracted from GLY04.2 consistent with expected Gal₂GlcNAc₂Man₃GlcNAc₂ (m/z 1662.2).

FIG. 8. Production of a NANAGalGlcNAcMan₃GlcNAc₂ glycoform. MALDI-TOF mass spectrum in negative ion mode of glycans synthesized ex vivo consistent with the expected NANAGalGlcNAcMan₃GlcNAc₂ (m/z 1565.7).

FIG. 9. Increased glycan yield. (A) Fluorophore-assisted carbohydrate electrophoresis (FACE) of lipid-released glycan extracted from E. coli ran with a Man₃GlcNAc₂ glycan standard (M3GN2 Std): with (GLY01.2) or without (GLY01) overexpression of ManC/B (left) consistent with the Man₃GlcNAc₂ glycoform and with (GLY01.3) or without (GLY01.1) the overexpression of GlmS (right) consistent with the GlcNAcMan₃GlcNAc₂ glycoform (GNM3GN2). (B) Quantity of lipid-released glycan extracted from GLY01.2 with overexpression of ManC/B and glycerol supplementation, as indicated. (C) FACE of lipid-released glycan extracted from GLY01.2 with either 0.2% glycerol or pyruvate supplementation.

FIG. 10. Increased product formation. MADLI-TOF mass spectra of lipid-released glycans extracted from strain (A) GLY01, (B) GLY02.3, and (C) GLY01.1 without overexpression of ManC/B and (D) GLY01.2, (E) GLY02.1, and (F) GLY01.5 with overexpression of ManC/B. The loss of peaks corresponding to intermediate glycoforms was observed with the addition of ManC/B.

FIG. 11. Glycosylated glucagon production. MALDI-TOF MS of partially purified glucagon appended with a C-terminal glycosylation site from various glycoengineered strains, which produce M3, M5, GlcNAcMan₃GlcNAc₂, and GalGlcNAcMan₃GlcNAc₂ glycopeptides.

FIG. 12. Glycosylated antigens. Western blot of partially purified (A) MBP-3473 and (B) MBP-1275 proteins originally from extraintestinal pathogenic E. coli (ExPEC) appended with four consecutive C-terminal glycosylation sites and expressed in GLY01 detected with anti-hexahistidine antibody (“hexahistidine” disclosed as SEQ ID NO: 35) (left) and the Concanavalin A lectin specific for terminal alpha-mannose (right). Figure discloses “6×His” as SEQ ID NO: 35.

### DETAILED DESCRIPTION OF THE INVENTION

### Definitions

The following definitions of terms and methods are provided to better describe the present disclosure and to guide those of ordinary skill in the art in the practice of the present disclosure.

All publications, patents and other references mentioned herein are hereby incorporated by reference in their entireties.

EC numbers are established by the Nomenclature Committee of the International Union of Biochemistry and Molecular Biology (NC-IUBMB) (available at http://www.chem.qmul.ac.uk/iubmb/enzyme/). The EC numbers referenced herein are derived from the KEGG Ligand database, maintained by the Kyoto Encyclopedia of Genes and Genomics, sponsored in part by the University of Tokyo. Unless otherwise indicated, the EC numbers are as provided in the database as of March 2013.

The accession numbers referenced herein are derived from the NCBI database (National Center for Biotechnology Information) maintained by the National Institute of Health, U.S.A. Unless otherwise indicated, the accession numbers are as provided in the database as of March 2013.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The term “claim” in the provisional application is synonymous with embodiments or preferred embodiments.

As used herein, “comprising” means “including” and the singular forms “a” or “an” or “the” include plural references unless the context clearly dictates otherwise. For example, reference to “comprising a cell” includes one or a plurality of such cells. The term “or” refers to a single element of stated alternative elements or a combination of two or more elements, unless the context clearly indicates otherwise.

The term “human-like” with respect to a glycoproteins refers to proteins having attached N-acetylglucosamine (GlcNAc) residue linked to the amide nitrogen of an asparagine residue (N-linked) in the protein, that is similar or even identical to those produced in humans.

“N-glycans” or “N-linked glycans” refer to N-linked oligosaccharide structures. The N-glycans can be attached to proteins or synthetic glycoprotein intermediates, which can be manipulated further in vitro or in vivo. The predominant sugars found on glycoproteins are glucose (Glu), galactose (Gal), mannose (Man), fucose (Fuc), N-acetylgalactosamine (GalNAc), N-acetylglucosamine (GlcNAc), and sialic acid (e.g., N-acetyl-neuraminic acid (NeuAc or NANA)). Hexose (Hex) may also be found. N-glycans differ with respect to the number of branches (“antennae” or “arms”) comprising peripheral sugars (e.g., GlcNAc, galactose, fucose and sialic acid) that are added to the “triamannosyl core”. The term “triamannosyl core”, also referred to as “M3”, “M3GN2”, the “triamannose core”, the “pentasaccharide core” or the “paucimannose core” reflects Man₃GlcNAc₂ oligosaccharide structure where Manα1,3 arm and the Manα1,6 arm extends from the di-GlcNAc structure (GlcNAc₂):β1,4GlcNAc-β1,4GlcNAc. N-glycans are classified according to their branched constituents (e.g., high-mannose, complex or hybrid).

A “high-mannose” type N-glycan comprises four or more mannose residues on the di-GlcNAc oligosaccharide structure. “M4” reflects Man₄GlcNAc₂. “M5” reflects Man₅GlcNAc₂.

A “hybrid” type N-glycan has at least one GlcNAc residue on the terminal end of the α1,3 mannose (Man α1,3) arm of the trimannose core and zero or more mannoses on the α1,6 mannose (Man α1,3) arm of the trimannose core. The various N-glycans are also referred to as “glycoforms”. An example of a hybrid glycan is “GNM3GN2”, which is GlcNAcMan₃GlcNAc₂.

A “complex” type N-glycan typically has at least one GlcNAc residue attached to the Manα1,3 arm and at least one GlcNAc residue attached to the Manα1,6 arm of the trimannose core. Complex N-glycans may also have galactose or N-acetylgalactosamine residues that are optionally modified with sialic acid or derivatives (e.g., “Neu” refers to neuraminic acid and “Ac” refers to acetyl). Complex N-glycans may also have intrachain substitutions comprising “bisecting” GlcNAc and core fucose. Complex N-glycans may also have multiple antennae on the trimannose core, often referred to as “multiple antennary glycans” or also termed “multi-branched glycans,” which can be tri-antennary tetra-antennary or penta-antennary glycans.

The term “G0” refers to GlcNAc₂Man₃GlcNAc₂. The term “G0(1)” refers to GlcNAc₃Man₃GlcNAc₂, the term “G0(2)” refers to GlcNAc₄Man₃GlcNAc₂ and the term “G0(3)” refers to GlcNAc₅Man₃GlcNAc₂. The terms “G1” refers to GalGlcNAc₂Man₃GlcNAc₂, “G2” refers to Gal₂GlcNAc₂Man₃GlcNAc₂, “G3” refers to Gal₃GlcNAc₃₋₅Man₃GlcNAc₂, “G4” refers to Gal₄GlcNAc₄₋₅Man₃GlcNAc₂, “G5” refers to Gal₅GlcNAc₅Man₃GlcNAc₂. The terms “S1” refers to NANAGal₁₋₅GlcNAc₁₋₅Man₃GlcNAc₂, “S2” refers to NANA₂Gal₂₋₅GlcNAc₂₋₅Man₃GlcNAc₂. “S3” refers to NANA₃Gal₃₋₅GlcNAc₃₋₅Man₃GlcNAc₂, “S4” refers to NANA₄Gal₄₋₅GlcNAc₄₋₅Man₃GlcNAc₂, “S5” refers to NANA₅Gal₅GlcNAc₅Man₃GlcNAc₂.

As used herein, the term “predominantly” or variations such as “the predominant” or “which is predominant” will be understood to mean the glycan species as measured that has the highest mole percent (%) of total N-glycans after the glycoprotein has been removed (e.g., treated with PNGase and the glycans released) and are analyzed by mass spectroscopy, for example, MALDI-TOF MS. In other words, the phrase “predominantly” is defined as an individual entity, such as a specific glycoform, present in greater mole percent than any other individual entity. For example, if a composition consists of species A in 40 mole percent, species B in 35 mole percent and species C in 25 mole percent, the composition comprises predominantly species A. The term “enriched”, “uniform”, “homogenous” and “consisting essentially of” are also synonymous with predominant in reference to the glycans.

The mole % of N-glycans as measured by MALDI-TOF-MS in positive mode refers to mole % saccharide transfer with respect to mole % total N-glycans. Certain cation adducts such as K+ and Na+ are normally associated with the peaks eluted increasing the mass of the N-glycans by the molecular mass of the respective adducts.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” or “substantially pure” nucleic acid or polynucleotide (e.g., RNA, DNA, or a mixed polymer) or glycoprotein is one which is substantially separated from other cellular components that naturally accompany the native polynucleotide in its natural host cell, e.g., ribosomes, polymerases and genomic sequences with which it is naturally associated. The term embraces a nucleic acid, polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleic acid, polynucleotide or glycoprotein so described has itself been physically removed from its native environment. For instance, an endogenous nucleic acid sequence in the genome of an organism is deemed “isolated” if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous coding sequence is considered “isolated” if it contains an insertion, deletion, or a point mutation introduced artificially, e.g., by human intervention. An “isolated nucleic acid” also includes a nucleic acid integrated into a host cell chromosome at a heterologous site and a nucleic acid construct present as an episome. Moreover, an “isolated nucleic acid” can be substantially free of other cellular material or substantially free of culture medium when produced by recombinant techniques or substantially free of chemical precursors or other chemicals when chemically synthesized.

Glycosylation Engineering

Using the novel expression system and methods as provided herein, various aspects of the invention are provided for the production of high-mannose, hybrid and complex glycans through glycoengineering of prokaryotic host cells. One aspect of the present invention relates to a recombinant prokaryotic host comprising a biosynthetic pathway to express N-linked glycoproteins with structurally homogeneous human-like glycans. Applications of the present invention include improved biochemical and pharmacokinetic stability for therapeutic proteins. Additional embodiments provide methods and compositions for producing carbohydrate-conjugated vaccines capable of eliciting protective immunity in subjects. A rapid, microbial-based manufacturing process to produce safe and more effective glycoproteins and vaccines is an object of the present invention.

High-Mannose Type Glycan Production in Prokaryotes

Building from the trimannosyl core, the present invention provides methods for the recombinant expression of a mannosyltransferase enzyme to produce a high-mannose type glycan as shown in FIG. 1. In one embodiment, the method provides culturing a recombinant prokaryotic host cell to express one or more alpha-1,2-mannosyltransferase enzyme activities (EC 2.4.1.131) that catalyzes the transfer of one or two GDP-Mannose residues onto a trimannose oligosaccharide composition in a prokaryotic host cell. Example 3 describes expression of a α-1,2-mannosyltransferase enzyme activity (EC 2.4.1.131). Preferred α-1,2-mannosyltransferase enzyme activity is encoded by a S. cerevisiae alg11 fused to GST, a solubility enhancer. Table 1 lists a variety of solubility enhancers.

Accordingly, the invention provides a method of producing a high-mannose type oligosaccharide composition, said method comprising: culturing a recombinant prokaryotic host cell that produces an oligosaccharide composition having a terminal mannose residue to express one or more alpha-1,2-mannosyltransferase enzyme activity (EC 2.4.1.131) that catalyzes the transfer of a GDP-Mannose residue onto the terminal mannose residue, said culturing step carried out under conditions effective to produce an oligosaccharide composition having at least 4 mannose residues. In certain embodiments, the oligosaccharide composition comprises at least 2 additional mannose residues on the trimannose core. In preferred embodiments, vaccine candidates are recombinantly expressed in the prokaryotic host cell where they are N-linked to the M5 glycoform. The expected structure of the major glycoform shown in FIG. 1 is Manα1-2 Manα1-2Manα1-3(Manα1-6)-Manβ1-4-GlcNAcβ1-4-GlcNAc.

In the prokaryotic host cell of the invention, the glycosylation enzymes act on lipid-linked glycans prior to the glycosylation of the glycoprotein. In eukaryotes, the alpha-1,2-mannosyltransferase acts on the trimannose core glycan linked to dolichol pyrophosphate on the cytosolic side of the endoplasmic reticulum membrane. The Man₅GlcNAc₂-dolichol pyrophosphate is then flipped into the endoplasmic reticulum by an endogenous flippase enzyme that is highly specific for Man₅GlcNAc₂-dolichol pyrophosphate to ensure the complete assembly of the oligosaccharide prior to flipping (Sanyal S, Menon A K (2009) Specific transbilayer translocation of dolichol-linked oligosaccharides by an endoplasmic reticulum flippase. Proc Natl Acad Sci USA 106:767-772). In prokaryotes, it has been shown that the Man₃GlcNAc₂ lipid can be flipped (Valderrama-Rincon, et. al. “An engineered eukaryotic protein glycosylation pathway in Escherichia coli,” Nat. Chem. Biol. AOP (2012)) and there is no known specificity for flipping, jeopardizing assembly of the oligosaccharide beyond the trimannose core. Therefore, it is an object of the invention to produce a high-mannose type oligosaccharide composition including Man₇₋₉GlcNAc₂, Man₆GlcNAc, Man₅GlcNAc₂ and Man₄GlcNAc₂ in a prokaryotic system that transfers mannose residues onto the M3 oligosaccharide substrates and, furthermore, catalyzes the flipping activity of the oligosaccharides into the periplasm. In preferred embodiments, the host cell produces 50 mole % or more of the high-mannose type glycans.

GnT Expression in Prokaryotes

In certain aspects, a method is provided for producing an oligosaccharide composition, said method comprising: culturing a recombinant prokaryotic host cell that produces an oligosaccharide composition having a terminal mannose residue to express one or more N-acetylglucosaminyl transferase enzyme activity (EC 2.4.1.101; EC 2.4.1.143; EC 2.4.1.145; EC 2.4.1.155; EC 2.4.1.201) that catalyzes the transfer of a UDP-GlcNAc residue onto said terminal mannose residue, said culturing step carried out under conditions effective to produce an oligosaccharide composition having a terminal GlcNAc residue. In eukaryotes, N-acetylglucosaminyl transferases act on oligosaccharides that are covalently linked to asparagine residues of glycosylated proteins. In prokaryotes, oligosaccharides are produced independently of the protein glycosylation process jeopardizing the production of hybrid and complex oligosaccharides.

To produce a hybrid glycoform, UDP-GlcNAc residue is transferred onto the Manα1,3 arm of the trimannosyl core oligosaccharide structure, the acceptor substrate. In an exemplary embodiment, the invention provides a prokaryotic host cell transformed with a gene encoding N. tabacum GnTI fused to MBP a solubility enhancer in a host cell expressing Alg13, Alg14, Alg1 and Alg2. A hybrid glycoform GlcNAcMan₃GlcNAc₂ is produced as shown in FIG. 2A. The expected structure of the glycoform shown is β1-2-GlcNAcManα1-3(Manα1-6)-Manβ1-4-GlcNAcβ1-4-GlcNAc.

To produce a complex glycoform, UDP-GlcNAc residue is transferred onto both the Manα1,3 and Manα1,6 arm of the trimannosyl core oligosaccharide structure, the acceptor substrate. In this embodiment, a prokaryotic host cell is transformed with a gene encoding human GnTII fused to MBP in a host cell expressing Alg13, Alg14, Alg1, Alg2 and GnTI. A complex GlcNAc₂Man₃GlcNAc₂ (G0) glycoform is produced as shown in FIG. 3 and the expected structure is β1-2-GlcNAcManα1-3(β1-2-GlcNAc Manα1-6)-Manβ1-4-GlcNAcβ1-4-GlcNAc.

In further aspects of the invention, multiple-antennary glycans are produced. For instance, a prokaryotic host cell is transformed with a gene encoding bovine GnTIV fused to MBP in a host cell expressing Alg13, Alg14, Alg1, Alg2 and GnTI. FIG. 4A demonstrates GlcNAc₂Man₃GlcNAc₂ hybrid glycoform produced using the methods of the invention wherein two UDP-GlcNAc residues are transferred onto the Manα1,3 arm of the trimannosyl core. The expected structure of the glycoform shown is β1-2-GlcNAc(β1-2-GlcNAc) Manα1-3(Manα1-6)-Manβ1-4-GlcNAcβ1-4-GlcNAc.

In alternative embodiments, glycans can also be formed ex vivo, e.g., through enzymatic synthesis of oligosaccharides as described in Example 7. For instance FIG. 5A depicts a MS of complex, multiple-antennary glycans comprising GlcNAc₃Man₃GlcNAc₂ glycoform, which was produced by expressing GnTI, GnTII, GnTIV (ex vivo), Alg13, Alg14, Alg1 and Alg2 resulting in the transfer of two UDP-GlcNAc residues onto the Manα1,3 arm and one UDP-GlcNAc residue onto the Manα1,6 arm of the trimannosyl core oligosaccharide structure. FIG. 5B depicts a MS of complex, multiple-antennary glycans comprising GlcNAc₃Man₃GlcNAc₂ glycoform, which was produced recombinantly in GLY06.4. The expected structure of the glycoform shown is (β1-2-GlcNAcManα1-3)β1-2-GlcNAc(β1-2-GlcNAc Manα1-6)-Manβ1-4-GlcNAcβ1-4-GlcNAc.

Additional GnT activities such as GnTV (EC 2.4.1.155) and GnTVI (2.4.1.201) can be expressed in the prokaryotic system. As a result, multiple antennary glycans of up to 5 branches on the trimannose core are possible using the methods of the invention. Multiple branched glycans enable, for example, enhanced sialylation on erythropoietin, increasing serum half-life and potentcy (Elliot, Nature Biotech 2003; Misaizu, Blood 1995). Glycosyltransferase Solubility Enhancers

While various GnTs can be expressed in a host cell, in preferred embodiments, GnTs are fused to, for example, MBP and expressed as a fusion protein to transfer a terminal UDP-GlcNAc residue onto the trimannosyl core, in effect, enhancing solubility of the glycosyltransferase. Table 1 provides a list of membrane targeting domains and solubility enhancers.

TABLE 1
Solubility Enhancers

| FUSION | Glycan Synthesis | |
|---|---|---|
| PARTNER | Alg11 | GnTI |
| None | − | − |
| DsbA | − | + |
| GlpF | +/− | + |
| GST | + | + |
| MBP | +/− | + |
| (EC# P0AEX9) | | |
| MstX | + | + |
| NusA | − | N/A |
| TrxA | − | N/A |

Using a library of fusions, glycans such as GlcNAc₍₁₋₅₎Man₃GlcNAc₂ are produced in the prokaryotic system of the present invention. In certain aspects of the invention, MBP-fused glycosyltransferases are expressed in a prokaryotic host. Other membrane targeting domains and solubility enhancers, such as MstX can also be expressed. Such N-acetylglucosaminyl transferase-MBP or N-acetylglucosaminyl transferase-MstX fusions are screened for the addition of UDP-GlcNAc residue onto the acceptor oligosaccharide substrate. In preferred embodiments, the following fusions: N. tabacum GnTI-MBP, H. Sapiens GnTII-MBP, B. taurus GnT IV-MBP confer UDP-GlcNAc transfer onto the trimannosyl core. Accordingly, a library of GnT fusions can be made to produce hybrid, complex and multi-antennary glycans in prokaryotic host cells. Various GnT fusion constructs can be made using the methods of the present invention. Such fusion constructs are within the scope of invention and can be screened for better activity or enhanced solubility.

Galactosyltransferase Expression in Prokaryotes

In further aspects of the invention, a method is provided for producing an oligosaccharide composition, said method comprising: culturing the host cell to express one or more galactosyltransferase enzyme activity (EC 2.4.1.38, EC 2.7.8.18) that catalyzes the transfer of a UDP-Galactose residue onto said terminal GlcNAc residue, said culturing step carried out under conditions effective to produce an oligosaccharide composition having a terminal galactose residue. FIG. 6 depicts a MS of the hybrid glycoform GalGlcNAcMan₃GlcNAc₂ produced in E. coli. Example 5 describes expression of Helicobacter pylori β-1,4GalT in E. coli, which transfers a UDP-Galactose residue onto the GlcNAcMan₃GlcNAc₂ acceptor oligosaccharide.

To produce a hybrid galactosylated glycoform in a prokaryote, UDP-Galactose residue is transferred onto the β-1,2GlcNAcManα1,3 of the trimannosyl core and both β-1,2GlcNAcManα1,3 and β-1,2GlcNAcManα1,6 arms of the trimannosyl core for the complex glycoform. In such embodiments, a prokaryotic host cell is transformed with a gene encoding H. pylori GalT in a host cell expressing the Alg13, Alg14, Alg1, Alg2, GnTI and GnTII. Example 8 provides methods for producing a complex Gal₂GlcNAc₂Man₃GlcNAc₂ glycoform. FIG. 7B shows a peak at m/z 1662.2, which correlates with the mass of the complex galactosylated glycan Gal₂GlcNAc₂Man₃GlcNAc₂. Additional galactosylated glycoforms can be produced including: Gal₍₁₋₄₎GlcNAc₂Man₃GlcNAc₂. The expected structure of the hybrid terminal galactose glycan is β1-4Galβ1-2-GlcNAcManα1-3(Manα1-6)-Manβ1-4-GlcNAcβ1-4-GlcNAc and the complex terminal galactose glycan is β1-4Galβ1-2-GlcNAcManα1-3(β1-4Galβ1-2-GlcNAc Manα1-6)-Manβ1-4-GlcNAcβ1-4-GlcNAc.

Galactosyltransferases from various other organisms can be expressed, which include but are not limited to Helicobacter pylori, Neisseria meningitides, Neisseria gonorrhoeae, Leishmania donovani, Homo sapiens (GALT), Bos Taurus, Drosophia, melanogaster, Rattus norvegicus (GalT I), Mus musculus, Cricetulus griseus, Equus caballus, Macropus eugenii (4β-GalT), Danio rerio (GalT I) and Sus scrofa, Ovis aries.

In some embodiments, various galactosyltransferase enzyme activities are fused to solubility enhancers such as MBP or mstX and screened for addition of UDP-Galactose onto the acceptor oligosaccharide substrate. Unlike the GnTs, the human and bovine GalT-mstX fusions did not appear to transfer UDP-Galactose onto the terminal GlcNAc oligosaccharide substrate.

In more preferred embodiments, oxidative bacterial strains are used for the expression of H. pylori β-1,4-GalT.

In an exemplary embodiment, the following enzymes are expressed in a prokaryotic host: Alg13, Alg14, Alg1, Alg2, Nicotiana tabaccum GnTI, human GnTII, bovine GnTIV, Helicobacter pylori β-1,4GalT. The GnTs and the GalT are expressed in an oxidative bacterial host.

Sialyltransferase Expression in Prokaryotes

Full complex oligosaccharide structures end in a terminal sialic acid, e.g., NANA residues. Expression of sialyltransferases in prokaryotes has been a considerable interest. While several groups have undertaken the task of sialic acid transfer for glycoprotein production for many years, to date, no reports exist for production of sialic acid transfer to produce a human-like glycan in prokaryotes.

Accordingly, the present invention provides methods to produce oligosaccharide compositions by culturing a recombinant prokaryotic host to express one or more sialyltransferase enzyme activity (EC 2.4.99.4 and EC 2.4.99.1) that catalyzes the transfer of a CMP-NANA residue onto said terminal galactose residue, said culturing step carried out under conditions effective to produce an oligosaccharide composition having a terminal sialic acid residue. Various sialyltransferases are expressed using the methods of the invention, either in vivo or ex vivo. In one embodiment, an α-2,3 sialyltransferase (EC 2.4.99.4) is expressed in a host cell or in the culture medium. In further embodiments, an α-2,6 sialyltransferase (EC 2.4.99.1) is expressed in a host cell or in the culture medium.

In preferred embodiments, the following enzymes are expressed in a prokaryotic host: Alg13, Alg14, Alg1, Alg2, Nicotiana tabaccum GnTI, bovine GnTIV, Helicobacter pylori β-1,4-GalT and P. damselae ST6. The method allows for a combination of in vivo and ex vivo reactions that demonstrate the proper transfer of CMP-NANA onto the acceptor oligosaccharide substrates. As shown in FIG. 8, the hybrid sialylated glycoform is produced where the expected structure of the glycoform shown is 2,6NANAβ1-4Gal β1-2-GlcNAcManα1-3(Manα1-6)-Manβ1-4-GlcNAcβ1-4-GlcNAc.

Sugar Nucleotide Precursors

In yet other embodiments, the method provides for culturing the host cell to increase sugar nucleotide precursors. For instance, enzymes that catalyze GDP-Mannose synthesis are expressed in the system. Phosphomannomutase enzyme activity (ManB) (EC 5.4.2.8) and mannose-1-phosphate guanylyltransferase enzyme activity (ManC) (EC 2.7.7.13) are introduced in the host cell of the invention. FIG. 9A (left) shows increased production of the trimannosyl core when ManC/B is overexpressed.

In additional embodiments, a sufficient pool of glycosyl donors in the cytoplasm is generated. UDP-GlcNAc, the substrate for GnTI and GnTII, is naturally present in the E. coli cytoplasm but the host cell can be engineered for increased UDP-GlcNAc synthesis. In such embodiments, the method provides for culturing the host cell to increase UDP-GlcNAc by expressing one or more glutamine-fructose-6-phosphate transaminase enzyme activities: GlmS (EC 2.6.1.16), GlmU (EC 2.7.7.23 & EC 2.3.1.157), GlmM (EC 5.4.2.10), which catalyze UDP-GlcNAc synthesis. FIG. 9A (right) shows an increase in GlcNAcMan₃GlcNAc₂ when GlmS was overexpressed. Addition of glycerol with ManC/B results in increased glycan yield as shown in FIG. 9B. Pyruvate also appears to increase glycan yield as shown in FIG. 9C.

Overexpression of ManC/B had a dramatic effect on the homogeneity of the glycans produced as evidenced in FIG. 10. Overexpression of ManC/B appears to have removed the peaks that may be due to the incomplete nucleotide sugar transfer of the reaction. Accordingly, as demonstrated by the predominant M3 glycoform (D), the M5 glycoform (E) and the GNM3GN2 glycoform (F), the host cell of the invention is capable of controlling the precise glycoform produced.

In yeast, Bobrowicz et al., showed increased production of terminally galactosylated glycans Pichia through expression of UDP-galactose transporter, UDP-galactose 4-epimerase and β1,4GalT in P. pastoris. (Bobrowicz et al., Engineering of an artificial glycosylation pathway blocked in core oligosaccharide assembly in the yeast Pichia pastoris: production of complex humanized glycoproteins with terminal galactose. Glycobiology 2004 September; 14(9):757-66.). UDP-Galactose is also naturally present in the cytoplasm of E. coli, however studies have shown that the availability of UDP-Galactose can be increased by overexpression of UDP-Gal synthesis genes including uridylate kinase (pyrH), Glc-1-P uridyltransferase (galU), Gal-1-P uridyltransferase (galT), galactokinase (galK), and UDP-galactose epimerase (galE) (Chung, S., et al., Galactosylation and sialylation of terminal glycan residues of human immunoglobulin G using bacterial glycosyltransferases with in situ regeneration of sugar-nucleotides. Enzyme and Microbial Technology, 2005. 39(1): p. 60-66.). Thus, in preferred embodiments one or more genes selected from galETK, galU, and pyrH from E. coli K12 is cloned using yeast-based recombination and subsequently expressed in the host strain to ensure a sufficient UDP-Gal pool of glycosyl donor substrates for transfer of galactose onto the acceptor oligosaccharide composition.

The modulation of CMP-NANA levels has been shown in both yeast and insect cells. Hamilton et al. showed increased cellular CMP-NANA pool for successful sialic acid transfer in P. pastoris using CMP-sialic acid transporter, UDP-GlcNAc 2-epimerase/N-acetylmannosamine kinase, CMP-sialic acid synthase, N-acetylneuraminate-9-phosphate synthase, and sialyltransferase (Hamilton, S. R., et al., Production of complex human glycoproteins in yeast. Science, 301, 1244 (2003)). Lawrence et al., showed coexpression of cytidine monophosphate sialic acid synthase (CMP-SA) and sialic acid phosphate synthase (SAS) gene with N-acetylmannosamine feeding for increased CMP-SA substrate production insect cells (Lawrence et al., Cloning and expression of human sialic acid pathway genes to generate CMP-sialic acids in insect cells. Glycoconj J. 2001 March; 18(3):205-13). Only a select few host cells such as E. coli K1 has endogenous CMP-NANA mechanism, however, many prokaryotes lack the machinery to produce CMP-NANA and it is at least expected that increased CMP-NANA levels is required for proper sialylation in prokaryotes.

The successful expression of eukaryotic proteins, especially membrane proteins, in E. coli and other bacteria is a nontrivial task (Baneyx et al., “Recombinant Protein Folding and Misfolding in Escherichia coli,” Nat Biotechnol 22:1399-1408 ((2004)). Thus, consideration has to be given to numerous issues in order to achieve high expression yields of correctly folded and correctly localized proteins (e.g., insertion into the inner membrane). All of these factors collectively dictate whether the eukaryotic proteins will be functional when expressed inside E. coli cells.

Additional Glycoengineering

Host cells that lack certain enzyme activities are preferred, such host cells that do not express or are attenuated in certain enzymes that compete with sugar biosynthesis (e.g., mannosyltransferases). In a preferred embodiment, the method provides for culturing the host cell that is attenuated in GDP-D-mannose dehydratase enzyme activity (EC 4.2.1.47) as shown in Valderrama-Rincon et al. An E. coli strain that lack the gmd gene encoding GDP-mannose dehydratase (GMD) is constructed that would in effect increase the availability of the substrate for Alg1 and Alg2, GDP-mannose, which is converted to GDP-4-keto-6-deoxymannose by GMD as the first step in the synthesis of GDP-L-fucose (Ruffing, A. & Chen, R. R. Metabolic engineering of microbes for oligosaccharide and polysaccharide synthesis. Microb Cell Fact 5, 25 (2006)). Additional engineering of the host cell may be required to knock-out certain competing pathways.

Codon Optimization

In additional embodiments of the present invention, eukaryotic glycosyltransferases are codon optimized to overcome limitations associated with the codon usage bias between E. coli (and other bacteria) and higher organisms, such as yeast and mammalian cells. Codon usage bias refers to differences among organisms in the frequency of occurrence of codons in protein-coding DNA sequences (genes). A codon is a series of three nucleotides (triplets) that encodes a specific amino acid residue in a polypeptide chain. Codon optimization can be achieved by making specific transversion nucleotide changes, i.e. a purine to pyrimidine or pyrimidine to purine nucleotide change, or transition nucleotide change, i.e. a purine to purine or pyrimidine to pyrimidine nucleotide change. In some instances, the codon optimized polypeptide variants retain the same biological function as the uncodon optimized polypeptides. For expression in E. coli, one or more codons can be optimized as described in, e.g., Grosjean et al., Gene 18:199-209 (1982). As used herein, “*” indicate stop codons.

The nucleic acid molecules, polypeptide molecules and homologs, variants and derivatives of the alg, N-acetylglucosaminyl transferase, galactosyltransferase, sialyltransferase, ManB/C, glmS, oligosaccharyl transferase described herein also comprise polynucleotide and polypeptide variants, which can be naturally occurring or created in vitro including chemical synthesis using known genetic engineering techniques. In some embodiments, the polynucleotide sequences have at least 75%, 77%, 80%, 85%, 90%, or 95% identity to SEQ ID NO:1, 3, 5, 7, 9, 11, 13, 15, 17, 19, 21, 23, 25, 27, 29, 31, 32, 33 or 34. In other embodiments, polypeptide variants have at least about 50%, 55%, 60%, 65%, 70%, 75%, 77%, 80%, 85%, 90%, or 95% homology to SEQ ID NO:2, 4, 6, 8, 10, 12, 14, 16, 18, 20, 22, 24, 26, 28 or 30.

The present invention also encompasses nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing can be performed at temperatures about 5° C. lower than the Tₘ for the specific DNA hybrid under a particular set of conditions.

The polynucleotides or nucleic acid molecules of the present invention refer to the polymeric form of nucleotides of at least 10 bases in length. These include DNA molecules (e.g., linear, circular, cDNA, chromosomal, genomic, or synthetic, double stranded, single stranded, triple-stranded, quadruplexed, partially double-stranded, branched, hair-pinned, circular, or in a padlocked conformation) and RNA molecules (e.g., tRNA, rRNA, mRNA, genomic, or synthetic) and analogs of the DNA or RNA molecules of the described as well as analogs of DNA or RNA containing non-natural nucleotide analogs, non-native inter-nucleoside bonds, or both. The isolated nucleic acid molecule of the invention includes a nucleic acid molecule free of naturally flanking sequences (i.e., sequences located at the 5′ and 3′ ends of the nucleic acid molecule) in the chromosomal DNA of the organism from which the nucleic acid is derived. In various embodiments, an isolated nucleic acid molecule can contain less than about 10 kb, 5 kb, 4 kb, 3 kb, 2 kb, 1 kb, 0.5 kb, 0.1 kb, 50 bp, 25 bp or 10 bp of naturally flanking nucleotide chromosomal DNA sequences of the microorganism from which the nucleic acid molecule is derived.

The heterologous nucleic acid molecule is inserted into the expression system or vector in proper sense (5′→3′) orientation relative to the promoter and any other 5′ regulatory molecules, and correct reading frame. The preparation of the nucleic acid constructs can be carried out using standard cloning methods well known in the art, as described by Sambrook et al., Molecular Cloning: A Laboratory Manual, Cold Springs Laboratory Press, Cold Springs Harbor, N.Y. (1989). U.S. Pat. No. 4,237,224 to Cohen and Boyer, also describes the production of expression systems in the form of recombinant plasmids using restriction enzyme cleavage and ligation with DNA ligase.

Suitable expression vectors include those which contain replicon and control sequences that are derived from species compatible with the host cell. For example, if E. coli is used as a host cell, plasmids such as pUC19, pUC18, or pBR322 may be used. Other suitable expression vectors are described in Molecular Cloning: a Laboratory Manual: 3rd edition, Sambrook and Russell, 2001, Cold Spring Harbor Laboratory Press. Many known techniques and protocols for manipulation of nucleic acids, for example in preparation of nucleic acid constructs, mutagenesis, sequencing, introduction of DNA into cells and gene expression, and analysis of proteins, are described in detail in Current Protocols in Molecular Biology, Ausubel et al. eds., (1992).

Different genetic signals and processing events control many levels of gene expression (e.g., DNA transcription and messenger RNA (“mRNA”) translation) and subsequently the amount of fusion protein that is displayed on the ribosome surface. Transcription of DNA is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, it is desirable to use strong promoters to obtain a high level of transcription and, hence, expression and surface display. Therefore, depending upon the host system utilized, any one of a number of suitable promoters may also be incorporated into the expression vector carrying the deoxyribonucleic acid molecule encoding the protein of interest coupled to a stall sequence. For instance, when using E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Translation of mRNA in prokaryotes depends upon the presence of the proper prokaryotic signals, which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979).

Host Cells

In accordance with the present invention, the host cell is a prokaryote. Such cells serve as a host for expression of recombinant proteins for production of recombinant therapeutic proteins of interest. Exemplary host cells include E. coli and other Enterobacteriaceae, Escherichia sp., Campylobacter sp., Wolinella sp., Desulfovibrio sp. Vibrio sp., Pseudomonas sp. Bacillus sp., Listeria sp., Staphylococcus sp., Streptococcus sp., Peptostreptococcus sp., Megasphaera sp., Pectinatus sp., Selenomonas sp., Zymophilus sp., Actinomyces sp., Arthrobacter sp., Frankia sp., Micromonospora sp., Nocardia sp., Propionibacterium sp., Streptomyces sp., Lactobacillus sp., Lactococcus sp., Leuconostoc sp., Pediococcus sp., Acetobacterium sp., Eubacterium sp., Heliobacterium sp., Heliospirillum sp., Sporomusa sp., Spiroplasma sp., Ureaplasma sp., Erysipelothrix, sp., Corynebacterium sp. Enterococcus sp., Clostridium sp., Mycoplasma sp., Mycobacterium sp., Actinobacteria sp., Salmonella sp., Shigella sp., Moraxella sp., Helicobacter sp, Stenotrophomonas sp., Micrococcus sp., Neisseria sp., Bdellovibrio sp., Hemophilus sp., Klebsiella sp., Proteus mirabilis, Enterobacter cloacae, Serratia sp., Citrobacter sp., Proteus sp., Serratia sp., Yersinia sp., Acinetobacter sp., Actinobacillus sp. Bordetella sp., Brucella sp., Capnocytophaga sp., Cardiobacterium sp., Eikenella sp., Francisella sp., Haemophilus sp., Kingella sp., Pasteurella sp., Flavobacterium sp. Xanthomonas sp., Burkholderia sp., Aeromonas sp., Plesiomonas sp., Legionella sp. and alpha-proteobacteria such as Wolbachia sp., cyanobacteria, spirochaetes, green sulfur and green non-sulfur bacteria, Gram-negative cocci, Gram negative bacilli which are fastidious, Enterobacteriaceae-glucose-fermenting gram-negative bacilli, Gram negative bacilli-non-glucose fermenters, Gram negative bacilli-glucose fermenting, oxidase positive.

In one embodiment of the present invention, the E. coli host strain C41(DE3) is used, because this strain has been previously optimized for general membrane protein overexpression (Miroux et al., “Over-production of Proteins in Escherichia coli: Mutant Hosts That Allow Synthesis of Some Membrane Proteins and Globular Proteins at High Levels,” J Mol Biol 260:289-298 (1996). Further optimization of the host strain includes deletion of the gene encoding the DnaJ protein (e.g., ΔdnaJ cells). The reason for this deletion is that inactivation of dnaJ is known to increase the accumulation of overexpressed membrane proteins and to suppress the severe cytotoxicity commonly associated with membrane protein overexpression (Skretas et al., “Genetic Analysis of G Protein-coupled Receptor Expression in Escherichia coli: Inhibitory Role of DnaJ on the Membrane Integration of the Human Central Cannabinoid Receptor,” Biotechnol Bioeng (2008)). Applicants have observed this following expression of Alg1 and Alg2. Furthermore, deletion of competing sugar biosynthesis reactions is required to ensure optimal levels of N-glycan biosynthesis. For instance, the deletion of genes in the E. coli O16 antigen biosynthesis pathway (Feldman et al., “The Activity of a Putative Polyisoprenol-linked Sugar Translocase (Wzx) Involved in Escherichia coli O Antigen Assembly is Independent of the Chemical Structure of the O Repeat,” J Biol Chem 274:35129-35138 (1999)) will ensure that the bactoprenol-GlcNAc-PP substrate is available for desired mammalian N-glycan reactions. To eliminate unwanted side reactions, the following are representative genes that are deleted from the E. coli host strain: wbbL, glcT, glf, gafT, wzx, wzy, waaL. Yet other strains include MC4100, BL21, ORIGAMI™, Shuffle®.

Methods for transforming/transfecting host cells with expression vectors are well-known in the art and depend on the host system selected, as described in Sambrook et al., Molecular Cloning: A Laboratory Manual, Cold Springs Laboratory Press, Cold Springs Harbor, N.Y. (1989). For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation, and transfection using bacteriophage.

A key advantage of the prokaryotic host cell of invention includes: (i) the massive volume of data surrounding the genetic manipulation of bacteria; (ii) the established track record of using bacteria for protein production ˜30% of protein therapeutics approved by the FDA since 2003 are produced in E. coli bacteria; and (iii) the existing infrastructure within numerous companies for bacterial production of protein drugs.

In comparison to various eukaryotic protein expression systems, the process employed using the methods and composition of the invention provides a scalable, cost-effective, optimal recombinant glycoprotein expression, free of human pathogens, free of immunogenic N- and O-linked glycosylation reactions, capable of rapid cloning and fast growth rate, fast doubling time (˜20 minutes), high growth (high OD), high titer and protein yields (in the range of 50% of the total soluble protein (TSP)), ease of product purification from the periplasm or supernatant, genetically tractable, thoroughly studied, compatible with the extensive collection of expression optimization methods (e.g., promoter engineering, mRNA stabilization methods, chaperone coexpression, protease depletion, etc.).

Another major advantage of prokaryotes, e.g., E. coli as a host for glycoprotein expression is that, unlike yeast and all other eukaryotes, there are no native glycosylation systems. Thus, the addition (or subsequent removal) of glycosylation-related genes is expected to have little to no bearing on the viability of glycoengineered E. coli cells. Furthermore, the potential for non-human glycan attachment to target proteins by endogenous glycosylation reactions is essentially eliminated in these cells.

Accordingly, in various embodiments, an alternative for glycoprotein expression and production of various oligosaccharide compositions (e.g., high-mannose, hybrid, complex) is disclosed where a prokaryotic host cell is used to produce the same and produce N-linked glycoproteins, which provide an attractive solution for circumventing the significant hurdles associated with eukaryotic cell culture. The use of bacteria as a production vehicle that yields structurally homogeneous human-like N-glycans while at the same time dramatically lowering the cost and time associated with protein drug development and manufacturing is an object of the invention.

Site-Specific Transfer of Oligosaccharide onto Target Proteins in Prokaryotes

As described in Valderrama-Rincon et al., to begin “humanizing” the bacterial glycosylation machinery, the Man₃GlcNAc₂ oligosaccharide structure is generated via a recombinant pathway comprising lipid-linked biosynthesis in E. coli. Specifically, one of several eukaryotic glycosyltransferases is functionally expressed in E. coli and the resulting lipid-linked oligosaccharides are transferred onto a protein via an oligosaccharyl transferase. Glycan assembly in the prokaryotic host cells is lipid-linked on undecaprenyl phosphate (Und-P) unlike eukaryotes where they are assembled on dolichol phosphate (Dol-P). In C. jejuni, N-linked glycosylation proceeds through the sequential addition of nucleotide-activated sugars onto a lipid carrier, resulting in the formation of a branched heptasaccharide. This glycan is then flipped across the inner membrane by PglK (formerly WlaB) and the OTase PglB then catalyzes the transfer of the glycan to an asparagine side chain. Bac is 2,4-diacetamido-2,4,6-trideoxyglucose; GalNAc is N-acetylgalactosamine; HexNAc is N-acetylhexosamine; Glc is glucose. See Szymanski et al., “Protein Glycosylation in Bacterial Mucosal Pathogens,” Nat Rev Microbiol 3:225-37 (2005). The PglK flippase is responsible for translocating the lipid-linked C. jejuni heptasaccharide across the inner membrane. Fortuitously, PglK exhibits relaxed specificity towards the glycan structure of the lipid-linked oligosaccharide intermediate (Alaimo et al., “Two Distinct But Interchangeable Mechanisms for Flipping of Lipid-linked Oligosaccharides,” Embo J 25:967-76 (2006) and Wacker et al., “Substrate Specificity of Bacterial Oligosaccharyltransferase Suggests a Common Transfer Mechanism for the Bacterial and Eukaryotic Systems,” Proc Natl Acad Sci USA 103:7088-93 (2006).

In preferred embodiments, the host cell of the invention expresses a flippase enzyme activity (Genbank AN AP009048.1), which translocates the undecaprenol-linked oligosaccharide across the inner membrane. Such enzyme activity may be endogenous or heterologous or engineered to be modified in expression. In additional embodiments, the prokaryotic host cell comprises a flippase activity including pglK and rftl.

Production of a human-like oligosaccharide structure in prokaryotes entails the transfer of various oligosaccharides to N-X-S/T sites on polypeptide chains. This requires functional expression of an integral membrane protein or protein complex known as an oligosaccharyltransferase (OST) that is responsible for the transfer of oligosaccharides to the target protein. Various prokaryotic and eukaryotic OSTs have the ability to transfer the lipid-linked oligosaccharide onto the target protein. The present invention discloses a prokaryotic system that demonstrates the transfer of high-mannose, hybrid and complex glycans onto a protein. Accordingly, the prokaryotic protein expression system comprises at least one OST activity to produce a glycosylated target protein. In such embodiments, the host cell expresses an oligosaccharyl transferase enzyme activity (EC 2.4.1.119) in addition to the glycosyltransferase enzymes. Various OSTs (Table 2) can be expressed and may be endogenous or heterologous or engineered to be modified in expression. In further embodiments, the prokaryotic host cell comprises at least one oligosaccharyl transferase activity, such as PglB from C. jejuni (Aebi et al.) or C. lari (Valderrama-Rincon et al.). The oligosaccharide transferred onto the protein is N-linked to the protein.

TABLE 2
List of Oligosaccharyltransferases.

| Protein | | |
|---|---|---|
| EC # | Organism | Gen Bank |
| CCC13826_0460 | Campylobacter concisus 13826 | EAT99324.2 |
| CFF8240_1383 | Campylobacter fetus subsp. fetus 82-40 | ABK82109.1 |
| CHAB381_0954 | Campylobacter hominis ATCC BAA-381 | ABS52339.1 |
| OrfA (fragment) | Campylobacter jejuni NCTC 11351 | AAD09300.1 |
| WlaF | Campylobacter jejuni 81116 | CAA72355.1 |
| | | ABV52665.1 |
| WlaF | Campylobacter jejuni D450 | AAK97437.1 |
| CJE1268 | Campylobacter jejuni RM1221 | AAW35590.1 |
| JJD26997_0595 | Campylobacter jejuni subsp. doylei | ABS43894.1 |
| | 269.97 | |
| OST (PglB; WlaF) | Campylobacter jejuni subsp. jejuni | AAK97438.1 |
| EC 2.4.1.119 | 81-176 | AAD51383.1 |
| OST | Campylobacter jejuni subsp. jejuni | CAB73381.1 |
| (PglB; WlaF, | NCTC 11168 | NP_282274.1 |
| Cj1126c) | | CAL35243.1 |
| EC 2.4.1.119 | | AAD09293.1 |
| Cla_1253 (PglB) | Campylobacter lari RM2100 RM2100; | ACM64573.1 |
| | ATCC BAA-1060D | |
| Ddes_0746 | Desulfovibrio desulfuricans subsp. | ACL48654.1 |
| | desulfuricans str. ATCC 27774 | |
| Dde_3699 | Desulfovibrio desulfuricans subsp. | ABB40492.1 |
| | desulfuricans str. G20 | |
| DvMF_0846 | Desulfovibrio vulgaris str. ‘Miyazaki F’ | ACL07802.1 |
| Dvul_1810 | Desulfovibrio vulgaris DP4 | ABM28827.1 |
| DVU1252 | Desulfovibrio vulgaris str. | AAS95730.1 |
| | Hildenborough | |
| Geob_1424 | Geobacter sp. FRC-32 | ACM19784.1 |
| Geob_2990 | | |
| NAMH_1652 | Nautilia profundicola AmH | ACM92784.1 |
| NIS_1250 | Nitratiruptor sp. SB155-2 | BAF70358.1 |
| Tmden_1474 | Sulfurimonas denitrificans DSM 1251 | ABB44751.1 |
| SUN_0103 | Sulfurovum sp. NBC37-1 | BAF71063.1 |
| WS0043 (WlaF) | Wolinella succinogenes DSM 1740 | CAE09214.1 |
| | | NP_906314.1 |
| OST, STT3 | Campylobacterales bacterium GD 1 | EDZ62411.1 |
| subunit | | |
| BACPLE_02950 | Bacteroides plebeius DSM 17135 | EDY94544.1 |
| BACPLE_02943 | Bacteroides plebeius DSM 17135 | EDY94539.1 |
| RHECIAT_ | Rhizobium etli CIAT 652 | ACE91723.1 |
| CH0002772 | | |
| BACINT_01142 | Bacteroides intestinalis DSM 17393 | EDV06057.1 |
| IMP (possible | Hydrogenivirga sp. 128-5-R1-1 | EDP74595.1 |
| OST) | | |
| OST (PglB) | Campylobacter coli RM2228 | EAL57053.1 |
| OST (PglB) | Campylobacter upsaliensis RM3195 | EAL53100.1 |

Oligosaccharide Compositions

Recently, several eukaryotic expression hosts have been introduced as alternatives to mammalian cell culture for making N-glycoproteins. These include the genetically engineered yeast Pichia pastoris (Hamilton, S. R., et al., Humanization of yeast to produce complex terminally sialylated glycoproteins. Science, 2006. 313(5792): p. 1441-3), cultured insect cells as hosts for recombinant baculovirus (Aumiller, J. J., J. R. Hollister, and D. L. Jarvis, A transgenic insect cell line engineered to produce CMP-sialic acid and sialylated glycoproteins. Glycobiology, 2003. 13(6): p. 497-507), and plant cells (Aviezer, D., et al., A plant-derived recombinant human glucocerebrosidase enzyme—a preclinical and phase I investigation. PLoS One, 2009. 4(3): p. e4792). Unfortunately, nonhuman glycoforms arise from native glycosylation pathways when using any eukaryotic host cell including mammalian, plant, insect, and yeast cells. Mammalian host cells have been shown to add uncontrollable levels of mannose-6-phosphate and fucose to glycans and often lack terminal sialic acid (Van Patten, S. M., et al., Effect of mannose chain length on targeting of glucocerebrosidase for enzyme replacement therapy of Gaucher disease. Glycobiology, 2007. 17(5): p. 467-78.). Plant cells add immunogenic beta-1,2 xylose and core alpha-1,3 fucose (Bardor, M., et al., Immunoreactivity in mammals of two typical plant glyco-epitopes, core alpha(1,3)-fucose and core xylose. Glycobiology, 2003. 13(6): p. 427-34), the latter is also found in insect cells (Bencurova, M., et al., Specificity of IgG and IgE antibodies against plant and insect glycoprotein glycans determined with artificial glycoforms of human transferrin. Glycobiology, 2004. 14(5): p. 457-66). O-linked glycosylation is also an essential process in yeast (Gentzsch, M. and W. Tanner, The PMT gene family: protein O-glycosylation in Saccharomyces cerevisiae is vital. Embo J, 1996. 15(21): p. 5752-9) and undesired O-glycans can be covalently attached to target glycoproteins.

The oligosaccharide chain attached by the prokaryotic glycosylation machinery is structurally distinct from that attached by higher eukaryotic and human glycosylation pathways (Weerapana et al., “Asparagine-linked Protein Glycosylation: From Eukaryotic to Prokaryotic Systems,” Glycobiology 16:91R-101R (2006)). The oligosaccharide compositions produced in the prokaryotes and from the methods of the present invention are also distinguishable from eukaryotic systems such as yeast, insect, mammalian and even human cells.

Several features distinguish oligosaccharide compositions produced by the methods of the invention in comparison to eukaryotic host cell expression systems, e.g., CHO, NS0, lemna, carrot, tobacco, Sf9. For instance, the oligosaccharide compositions of the present invention lack fucose. The absence of fucose in antibodies has been associated with increased ADCC and CDC activities (Shinkawa T et al., The absence of fucose but not the presence of galactose or bisecting N-acetylglucosamine of human IgG1 complex-type oligosaccharides shows the critical role of enhancing antibody-dependent cellular cytotoxicity. J Bio Chem, 278, 3466-73, 2003). Furthermore, prokaryotes inherently lack O-linked glycans, which is associated with immunogenicity. The oligosaccharide compositions of the present invention do not express abhorrent glycans that are present in many eukaryotic expression systems such as high-mannose or mannose phosphates. In addition, glycoengineered E. coli provides (i) control of the specific site and stoichiometry of glycosylation including at the N- or C-terminus, (ii) selection of the glycoform (iii) ability to engineer novel glycoforms because glycosylation is not an essential process in E. coli, and (iv) lack of competing glycosylation pathways including O-glycosylation and mannose 6-phosphate which improves product uniformity and may help avoid mislocalization to other receptors within the human host such as the mannose 6-phosphate receptor (Hayette, M. P. et al. Presence of human antibodies reacting with Candida albicans O-linked oligomannosides revealed by using an enzyme-linked immunosorbent assay and neoglycolipids. J Clin Microbiol 30, 411-417 (1992). Podzorski, R. P., Gray, G. R. & Nelson, R. D. Different effects of native Candida albicans mannan and mannan-derived oligosaccharides on antigen-stimulated lymphoproliferation in vitro. J Immunol 144, 707-716 (1990).).

The oligosaccharide compositions of the present invention can be uniform and also be enriched so as to boost anti-inflammatory properties, e.g., enriching for α2,6 sialic acid on Fc of intravenous Ig (IVIG) (Anthony et al., Identification of a receptor required for the anti-inflammatory activity of IVIG. Natl Acad Sci USA 2008 Dec. 16; 105(50):19571-8). Additional studies have indicated the presence of Neu5Gc-specific antibodies in all humans, sometimes at high levels (Ghaderi et al., Implications of the presence of N-glycolylneuraminic acid in recombinant therapeutic glycoproteins. Nat Biotechnol, 2010 August; 28(8): 863-7). Thus, enriching for therapeutic proteins, e.g., antibodies with specific sialic acid residues (e.g., NeuNAc as opposed to Neu5Ac, Neu5Gc) may reduce adverse reaction such as immunogenicity or inefficacy of protein therapeutics.

As reflected herein, the prokaryotic system can yield homogenous glycans at a relatively high yield. In preferred embodiments, the oligosaccharide composition consists essentially of a single glycoform in at least 50, 60, 70, 80, 90, 95, 99 mole %. In further embodiments, the oligosaccharide composition consists essentially of two desired glycoforms of at least 50, 60, 70, 80, 90, 95, 99 mole %. In yet further embodiments, the oligosaccharide composition consists essentially of three desired glycoforms of at least 50, 60, 70, 80, 90, 95, 99 mole %.

In certain embodiments, the oligosaccharide compositions produced are GlcNAc₁₋₅Man₃GlcNAc₂ and Man₃GlcNAc₂. Certain glyco-engineered host cells produce oligosaccharide composition that is predominantly GlcNAcMan₃GlcNAc₂ or GlcNAc₂Man₃GlcNAc₂.

In other embodiments, the oligosaccharide compositions produced are Gal₁₋₅GlcNAc₁₋₅Man₃GlcNAc₂ and Man₃GlcNAc₂. Certain glyco-engineered host cells produce oligosaccharide composition that is predominantly GalGlcNAcMan₃GlcNAc₂, GalGlcNAc₂Man₃GlcNAc₂ or Gal₂GlcNAc₂Man₃GlcNAc₂.

In yet other embodiments, the oligosaccharide compositions produced are NANA₁₋₅Gal₁₋₅GlcNAc₁₋₅Man₃GlcNAc₂. Certain glyco-engineered host cells produce oligosaccharide composition that is predominantly NANAGalGlcNAcMan₃GlcNAc₂ or NANA₂Gal₂GlcNAc₂Man₃GlcNAc₂.

In still other embodiments, the oligosaccharide compositions produced are Man₂GlcNAc₂, Man₄GlcNAc, Man₃GlcNAc₂, HexMan₃GlcNAc₂, HexMan₅GlcNAc Man₆GlcNAc and Man₅GlcNAc₂. Certain glycol-engineered host cells produce oligosaccharide composition that is predominantly Man₅GlcNAc₂.

The present invention, therefore, provides stereospecific biosynthesis of a vast array of novel oligosaccharide compositions and N-linked glycoproteins. In certain embodiments, reconstitution of a eukaryotic N-glycosylation pathway in E. coli using metabolic pathway and protein engineering techniques results in N-glycoproteins with structurally homogeneous human-like glycans. This ensures that each glycoengineered cell line corresponds to a unique carbohydrate signature.

The glycans can be analyzed by metabolic labeling of cells with ³H-GlcNAc and ³H-mannose or with fluorescent lectins (e.g., AlexaFluor-ConA). Glycans can also be released with PNGase and detected under MALDI/TOF-MS.

Quantification of the glycans can be estimated with the MS or more exactly done through HPLC. NMR can determine the glycosidic linkages of the glycan structures.

Target Glycoproteins

To produce various glycoproteins of interest, a gene encoding a target protein is introduced into the host cell.

“Target proteins”, “proteins of interest”, or “therapeutic proteins” include without limitation cytokines such as interferons, G-CSF, coagulation factors such as factor VIII, factor IX, and human protein C, soluble IgE receptor α-chain, IgG, IgG fragments, IgM, interleukins, urokinase, chymase, and urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II, α-feto proteins, AAT, rhTBP-1 (aka TNF binding protein 1), TACI-Ig (transmembrane activator and calcium modulator and cyclophilin ligand interactor), FSH (follicle stimulating hormone), GM-CSF, glucagon, glucagon peptides, GLP-1 w/ and w/o FC (glucagon like protein 1) IL-1 receptor agonist, sTNFr (aka soluble TNF receptor Fc fusion), CTLA4-Ig (Cytotoxic T Lymphocyte associated Antigen 4-Ig), receptors, hormones such as human growth hormone, erythropoietin, peptides, stapled peptides, human vaccines, animal vaccines, serum albumin and enzymes such as ATIII, rhThrombin, glucocerebrosidase and asparaginase.

Already approved therapeutics from E. coli are also target proteins. They include hormones (human insulin and insulin analogues, calcitonin, parathyroid hormone, human growth hormone, glucagons, somatropin and insulin growth factor 1), interferons (α1, α2a, α2b and γ1b), interleukins 2 and 11, light and heavy chains raised against vascular endothelial growth factor-α, tumor necrosis factor α, cholera B subunit protein, B-type natriuretic peptide, granulocyte colony stimulating factor and tissue plasminogen activator.

Target proteins also include a glycoprotein conjugate comprising a protein and at least one peptide comprising a D-X₁-N-X₂-T motif fused to the protein, wherein D is aspartic acid, X₁ and X₂ are any amino acid other than proline, N is asparagine, and T is threonine.

In preferred embodiments, at least 30, 50, 70, 90, 95 and preferably 100 mol % of glycans are transferred onto a target protein by an OST.

Culture Conditions

In other embodiments, the methods provide culturing the host cells under oxidative conditions. Preferably, an oxidative bacterial strain is used. Culture conditions may result in increased yield and titre of glycoproteins and glycans. Such process conditions and parameters include regulating pH, temperature, osmolality, culture duration, media, nutrients, concentration of dissolved oxygen, nitrogen, level or availability of nucleotide sugars and even carbon source, e.g., glycerol (FIG. 9B) can influence the production system. Culture conditions may vary depending on the product and the specific host cell utilized. Productivity of the system is also likely to be affected by the culture conditions. Additional metabolic engineering may be required for maximum or optimum productivity and to limit growth-inhibiting metabolites.

Enzymatic Synthesis of Oligosaccharides

In alternative aspects of the invention, glycans are synthesized in a cell-free extract using an acceptor glycan, purified enzyme/lysate and adding nucleotide sugars as described in Example 7.

In certain embodiments, the present invention provides a cell culture comprising a recombinant prokaryote, UDP-GlcNAc and a GnT (EC 2.4.1.101; EC 2.4.1.143; EC 2.4.1.145) wherein said GnT catalyzes the transfer of a UDP-GlcNAc residue onto said terminal mannose residue, cultured under conditions effective to produce an oligosaccharide composition having a terminal GlcNAc residue.

In further embodiments, the present invention provides a cell culture comprising a recombinant prokaryote, UDP-Galactose and a GalT (EC 2.4.1.38) wherein said GalT catalyzes the transfer of a UDP-Galactose residue onto said terminal GlcNAc residue, cultured under conditions effective to produce an oligosaccharide composition having a terminal galactose residue.

In preferred embodiments, the present invention provides a cell culture comprising a recombinant prokaryote, CMP-NANA and a sialyltransferase (EC 2.4.99.4 and EC 2.4.99.1) wherein said sialyltransferase catalyzes the transfer of a CMP-NANA residue onto said terminal galactose residue, cultured under conditions effective to produce an oligosaccharide composition having a terminal sialic acid residue.

Aglycosylated Vs. Glycosylated IgGs

Another aspect of the present invention relates to a glycosylated antibody comprising an Fv portion which recognizes and binds to a native antigen and an Fc portion which is glycosylated at a conserved asparagine residue. Alternative embodiments include diabody, scFv, scFv-Fc, scFv-CH, Fab and scFab.

The glycosylated antibody of the present invention can be in the form of a monoclonal or polyclonal antibody.

A single immunoglobulin molecule is comprised of two identical light (L) chains and two identical heavy (H) chains. Light chains are composed of one constant domain (C_{L}) and one variable domain (V_{L}) while heavy chains are consist of three constant domains (C_{H}1, C_{H}2 and C_{H}3) and one variable domain (V_{H}). Together, the V_{H} and V_{L} domains compose the antigen-binding portion of the molecule known as the Fv. The Fc portion is glycosylated at a conserved Asn297 residue. Attachment of N-glycan at this position results in an “open” conformation that is essential for effector interaction.

Monoclonal antibodies can be made using recombinant DNA methods, as described in U.S. Pat. No. 4,816,567 to Cabilly et al. and Anderson et al., “Production Technologies for Monoclonal Antibodies and their Fragments,” Curr Opin Biotechnol. 15:456-62 (2004). The polynucleotides encoding a monoclonal antibody are isolated, such as from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which are then transfected into the host cells of the present invention, and monoclonal antibodies are generated. In one embodiment, recombinant DNA techniques are used to modify the heavy and light chains with N-terminal export signal peptides (e.g., PelB signal peptide) to direct the heavy and light chain polypeptides to the bacterial periplasm. Also, the heavy and light chains can be expressed from either a bicistronic construct (e.g., a single mRNA that is translated to yield the two polypeptides) or, alternatively, from a two cistron system (e.g., two separate mRNAs are produced for each of the heavy and light chains). To achieve high-level expression and efficient assembly of full-length IgGs in the bacterial periplasm, both the bicistronic and two cistron constructs can be manipulated to achieve a favorable expression ratio. For example, translation levels can be raised or lowered using a series of translation initiation regions (TIRs) inserted just upstream of the bicistronic and two cistron constructs in the expression vector (Simmons et al., “Translational Level is a Critical Factor for the Secretion of Heterologous Proteins in Escherichia coli,” Nat Biotechnol 14:629-34 (1996)). When this antibody producing plasmid is introduced into a bacterial host that also harbors plasmid- or genome-encoded genes for expressing glycosylation enzymes, the resulting antibodies are glycosylated in the periplasm. Recombinant monoclonal antibodies or fragments thereof of the desired species can also be isolated from phage display libraries as described (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991)).

The polynucleotide(s) encoding a monoclonal antibody can further be modified in a number of different ways using recombinant DNA technology to generate alternative antibodies. In one embodiment, the constant domains of the light and heavy chains of, for example, a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

In some embodiments, the antibody of the present invention is a humanized antibody. Humanized antibodies are antibodies that contain minimal sequences from non-human (e.g. murine) antibodies within the variable regions. Such antibodies are used therapeutically to reduce antigenicity and human anti-mouse antibody responses when administered to a human subject. In practice, humanized antibodies are typically human antibodies with minimal to no non-human sequences. A human antibody is an antibody produced by a human or an antibody having an amino acid sequence corresponding to an antibody produced by a human.

Humanized antibodies can be produced using various techniques known in the art. An antibody can be humanized by substituting the complementarity determining region (CDR) of a human antibody with that of a non-human antibody (e.g. mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988)). The humanized antibody can be further modified by the substitution of additional residues either in the Fv framework region and/or within the replaced non-human residues to refine and optimize antibody specificity, affinity, and/or capability.

Bispecific antibodies are also suitable for use in the methods of the present invention. Bispecific antibodies are antibodies that are capable of specifically recognizing and binding at least two different epitopes. Bispecific antibodies can be intact antibodies or antibody fragments. Techniques for making bispecific antibodies are common in the art (Traunecker et al., “Bispecific Single Chain Molecules (Janusins) Target Cytotoxic Lymphocytes on HIV Infected Cells,” EMBO J. 10:3655-3659 (1991) and Gruber et al., “Efficient Tumor Cell Lysis Mediated by a Bispecific Single Chain Antibody Expressed in Escherichia coli,” J. Immunol. 152:5368-74 (1994)).

Glycosylated Glucagon Peptide Production in Prokaryotes

Simple in vitro glycoconjugation techniques have been demonstrated to improve glucagon peptides, however drawbacks of therapeutic such peptides still exist as they are small and generally monomeric, have short half-lives of generally less than a few hours and PEGylation very rarely works well with small peptides. Current approaches still suffer from activity that is significantly inhibited.

The present invention relates to novel glycosylated peptides with desired glycans. Advantages of glycosylated glucagon peptide include improved solubility, improved physical stability toward gel and fibril formation, with increased half-life and improved activity and pharmacokinetic properties. Other advantages include the capability of a single or simultaneous in vivo process to produce both protein and glycans thereby avoiding multiple steps. In some embodiments, the novel glycosylated glucagon peptides have prolonged exposure in vivo due to prolonged plasma elimination half-life and a prolonged absorption phase and improved aqueous solubility at neutral pH or slightly basic pH. In other embodiments, the present invention has improved stability towards formation of gels and fibrils in aqueous solutions. In preferred embodiments, the predominant N-glycan is one that does not illicit immunogenicity to mammals. N-glycosylation site occupancy can vary in eukaryotic systems, e.g., CHO and yeast for any particular glycoproteins produced. Growth conditions can be made to control occupancy at sites.

Typically, glucagon peptide has no glycosylation. In certain embodiments, glycosylation sites are engineered onto the peptide. In an exemplary embodiment, the glucagon peptide of the present invention has one glycosylation site. In certain embodiments, the method provides adding multiple glycans per peptide to confer better activity. In further embodiments, the host cells are engineered to produce glucagon peptides, with specific N-glycan as the predominant species. Exemplary glycosylation patterns are shown in FIG. 11.

Accordingly, the methods of the present invention provide glycoproteins and glycopeptides comprising one or more glycoforms. Preferably, the glycoforms include, for example, M4, M5, G0, G0(1), G0(2), G0(3), G1, G2, G3, G4, G5, S1, S2, S3, S4, S5 which confer improved solubility or stability properties as well as increased receptor binding activity. In comparison to aglycosylated peptides, such as glucagon, the present invention is expected to increase half-life for the peptide. Additional peptides have been produced by the methods of the prevention invention such as hGH, ASNase, and IL1-Ra. Production of other peptides are within the scope of the invention. In preferred embodiments, at least 50 mol % of glucagon peptide is glycosylated.

Vaccine Preparation

A generalized method to enhance immunogenicity of candidate antigens would reduce the time and costs invested in the early stages of vaccine development and could be applied to nearly any disease of interest. One documented strategy to enhance immunogenicity is mannosylation, the conjugation of mannose-terminal glycans to proteins. Mannose targets antigens to specific receptors including CD206 and CD209 on antigen presenting cells (APC) for internalization by receptor-mediated endocytosis resulting in up to a 200-fold increase in antigen presentation compared to antigens taken up via pinocytosis (Engering, A., et al., The mannose receptor functions as a high capacity and broad specificity antigen receptor in human dendritic cells. Eur J Immunol, 1997. 27(9): p. 2417-25. Lam, J. S., et al., A Model Vaccine Exploiting Fungal Mannosylation to Increase Antigen Immunogenicity. The Journal of Immunology, 2005. 175(11): p. 7496-7503.). Mannosylation of antigens confers several advantages including: (i) increased antigen uptake by APC, (ii) enhanced MHC class II-mediated antigen presentation by up to 10,000-fold, (iii) promotion of T cell proliferation and maturation, and (iv) improved humoral immune response including bactericidal activity of serum (Arigita, C., et al., Liposomal Meningococcal B Vaccination: Role of Dendritic Cell Targeting in the Development of a Protective Immune Response. Infection and Immunity, 2003. 71(9): p. 5210-5218.).

In certain embodiments, the present invention provides methods and compositions for mannosylated vaccine antigens through glycoengineered strains of E. coli. The effect of mannosylation on immunogenicity is assessed in a mouse model. The ability to produce vaccine candidates in bacteria provides multiple advantages. E. coli is an excellent platform for expression of ExPEC (extraintestinal pathogenic E. coli) and other bacterial proteins, offers facile recombinant DNA manipulation, can be used to generate large combinatorial libraries, allows for rapid and low cost strain development and quick ramp-up to production, and eliminates the risk for viral contamination encountered with eukaryotic expression systems (Aguilar-Yanez, J., et al., An influenza A/H1N1/2009 hemagglutinin vaccine produced in Escherichia coli. PLoS One, 2010. 5(7): p. e11694. Choi, B.-K., et al., Use of combinatorial genetic libraries to humanize N-linked glycosylation in the yeast Pichia pastoris. Proceedings of the National Academy of Sciences, 2003. 100(9): p. 5022-5027.). Production of mannosylated candidate antigens in E. coli would allow for synthesis of the desired glycoprotein in vivo without the need for further chemical or enzymatic modification. Accordingly, a vaccine development is provided by a method to augment the efficacy of E. coli-produced vaccine candidates.

Glycoengineered E. coli of the present invention is contemplated to produce mannosylated proteins with enhanced immunogenicity. Synthesis of mannosylated antigens in E. coli represents a significant advance in vaccine development allowing for inexpensive, rapid production of candidate proteins with enhanced immunogenic properties. In the past, several strategies have been employed for mannosylating antigens including in vitro chemical conjugation of mannan or mannose-terminal glycans, in vivo expression of proteins in Pichia pastoris for glycosylation with yeast high mannose oligosaccharides, or in vitro encapsulation of antigen in a mannosylated liposome (Lam, J. S., et al., Arigita, C., et al., Apostolopoulos, V., et al., Oxidative/reductive conjugation of mannan to antigen selects for T1 or T2 immune responses. Proceedings of the National Academy of Sciences, 1995. 92(22): p. 10128-10132. Sheng, K., et al., Delivery of antigen using a novel mannosylated dendrimer potentiates immunogenicity in vitro and in vivo. Eur J Immunol, 2008. 38(2): p. 424-36.). However, to date, the direct in vivo conjugation of mannose-terminal glycans to proteins in bacteria for vaccine development has never been achieved. An E. coli expression platform would provide multiple advantages over existing technologies both in terms of general protein production and an ectopic host for expression of glycans.

Extraintestinal E. coli (ExPEC) proteins c1275, and ECOK1_{—}3473 were selected for preliminary expression and glycosylation (Example 11). Candidate antigens are modified with the various oligosaccharides such as Man₃GlcNAc₂. This can result in generation of antigens modified with a eukaryotic mannose-terminal glycan for use in vaccine formulations. Numerous target antigens are selected from a published assessment of ExPEC vaccine candidates that are known to confer protection in a mouse model. It should be pointed out, however, that the invention is highly modular and thus could be widely applied to enhance vaccine development for a variety of protein and peptide candidates.

Pharmaceutical Formulations

Therapeutic formulations of the glycoprotein can be prepared by mixing the glycoprotein having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICST™ or polyethylene glycol (PEG).

A glycan is a convenient anchor for a PEG polymer because certain sugars, such as mannose or galactose, can easily be converted to reactive aldehydes in the presence of a mild oxidizer such as sodium periodate (Soares, A. L., et al., Effects of polyethylene glycol attachment on physicochemical and biological stability of E. coli L-asparaginase. Int J Pharm, 2002. 237(1-2): p. 163-70). A PEG polymer functionalized with a hydrazine group can then be used to create a glycoPEGylated bioconjugate. This allows the synthesis of site-specific, highly controlled, homogeneous, and active protein conjugates. PEGylation often results in problems of heterogeneity and activity loss as a result of the often non-specific process. Site-specific PEGylation methods involve either: (i) mutating lysine residues to allow PEG targeting to a specific lysine (Narimatsu, S., et al., Lysine-deficient lymphotoxin-alpha mutant for site-specific PEGylation. Cytokine, 2011. 56(2): p. 489-93. Youn, Y. S. and K. C. Lee, Site-specific PEGylation for high-yield preparation of Lys(21)-amine PEGylated growth hormone-releasing factor (GRF) (1-29) using a GRF (1-29) derivative FMOC-protected at Tyr(1) and Lys(12). Bioconjug Chem, 2007. 18(2): p. 500-6) or to the amine group of the N-terminus (Lee, H., et al., N-terminal site-specific mono-PEGylation of epidermal growth factor. Pharm Res, 2003. 20(5): p. 818-25. Yamamoto, Y., et al., Site-specific PEGylation of a lysine-deficient TNF-alpha with full bioactivity. Nat Biotechnol, 2003. 21(5): p. 546-52) or (ii) adding unpaired cysteine residues to allow targeting of free thiol groups (Shaunak, S., et al., Site-specific PEGylation of native disulfide bonds in therapeutic proteins. Nat Chem Biol, 2006. 2(6): p. 312-3. Doherty, D. H., et al., Site-specific PEGylation of engineered cysteine analogues of recombinant human granulocyte-macrophage colony-stimulating factor. Bioconjug Chem, 2005. 16(5): p. 1291-8. Manjula, B. N., et al., Site-specific PEGylation of hemoglobin at Cys-93(beta): correlation between the colligative properties of the PEGylated protein and the length of the conjugated PEG chain. Bioconjug Chem, 2003. 14(2): p. 464-72). These approaches have some major drawbacks. First, positively charged lysines are often important for protein structure/function (Yoshioka, Y., et al., Optimal site-specific PEGylation of mutant TNF-alpha improves its antitumor potency. Biochem Biophys Res Commun, 2004. 315(4): p. 808-14). Second, adding cysteine residues creates serious problems with soluble expression and disulphide bond formation, and can even require moving to a mammalian expression host (Constantinou, A., et al., Site-specific polysialylation of an antitumor single-chain Fv fragment. Bioconjug Chem, 2009. 20(5): p. 924-31). Third, site-specific PEGylation severely limits the number of linked PEG molecules. GlycoPEGylation, involves conjugation of PEG to glycans that are already attached to specific residues within proteins. The advantages are that: (i) the process is site-specific, (ii) glycosylation sites can be engineered away from the active site(s), and (iii) the product can be highly active and relatively homogeneous.

The formulation herein may also contain more than one active compound as necessary for the particular indication being treated, preferably those with complementary activities that do not adversely affect each other. For instance, the formulation may further comprise another antibody or a chemotherapeutic agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes. Sustained-release preparations may be prepared. Suitable examples of sustained-release preparations include semipermeable matrices of solid hydrophobic polymers containing the glycoprotein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The pharmaceutical composition may be lyophilized. Lyophilized antibody formulations are described in U.S. Pat. No. 6,267,958. Stable aqueous antibody formulations are described in U.S. Pat. No. 6,171,586B1.

As used herein, the term “therapeutically effective amount” of a therapeutic protein refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient or subject for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The patient to be treated is preferably a mammal, in particular a human being, but treatment of other animals, such as dogs, cats, cows, horses, sheep, goats or pigs, is within the scope of the invention.

For example, a therapeutically effective amount of glucagon peptide of the present invention for a patient suffering from insulin coma or insulin reaction resulting from severe hypoglycemia (low blood sugar) is 1 mg (1 unit) for an adult. For children weighing less than 441b (20 kg), it is 0.5 mg. Glucagon is given if (1) the patient is unconscious, (2) the patient is unable to eat sugar or a sugar-sweetened product, (3) the patient is having a seizure, or (4) repeated administration of sugar or a sugar-sweetened product such as a regular soft drink or fruit juice does not improve the patient's condition. In other instances, the dose can be in the range of 0.25 units to 2 units, which can be administered by intramuscular, intravenously or subcutaneous injection. A milligram of pure glucagon is approximately equivalent to 1 unit. A dosing schedule can vary but can be from about once a day to as needed per event. The actual schedule will depend on a number of factors including the type of glucagon administered to a patient (glucagon or glycosylated-glucagon) and the response of the individual patient. The higher dose ranges are not typically used in hypoglycemia applications but may be useful on other therapeutic applications. The means of achieving and establishing an appropriate dose for a patient is well known and commonly practiced in the art.

As used herein, the term “pharmaceutically acceptable” is given its ordinary meaning Pharmaceutically acceptable compositions are generally compatible with other materials of the formulation and are not generally deleterious to the subject.

Any of the compositions of the present invention may be administered to the subject in a therapeutically effective dose. For vaccines, a “therapeutically effective” or an “effective” amount or dose, as used herein means that amount necessary to induce immunity or tolerance within the subject, and/or to enable the subject to more effectively resist a disease (e.g., against foreign pathogens, cancer, an autoimmune disease, etc.). When administered to a subject, effective amounts will depend on the particular condition being treated and the desired outcome. A therapeutically effective dose may be determined by those of ordinary skill in the art, for instance, employing factors such as those further described below and using no more than routine experimentation.

In some embodiments, a therapeutically effective amount can be initially determined from cell culture assays. For instance the effective amount of a composition of the invention useful for inducing dendritic cell response can be assessed using the in vitro assays with respect to a stimulation index. The stimulation index can be used to determine an effective amount of a particular composition of the invention for a particular subject, and the dosage can be adjusted upwards or downwards to achieve desired levels in the subject. Therapeutically effective amounts can also be determined from animal models. The applied dose can be adjusted based on the relative bioavailability and potency of the administered composition. Adjusting the dose to achieve maximal efficacy based on the methods described above and other methods are within the capabilities of those of ordinary skill in the art. These doses can be adjusted using no more than routine experimentation.

In administering the compositions of the invention to a subject, dosing amounts, dosing schedules, routes of administration, and the like may be selected so as to affect known activities of these compositions. Dosages may be estimated based on the results of experimental models, optionally in combination with the results of assays of compositions of the present invention. Dosage may be adjusted appropriately to achieve desired compositional levels, local or systemic, depending upon the mode of administration. The doses may be given in one or several administrations per day. In the event that the response of a particular subject is insufficient at such doses, even higher doses (or effectively higher doses by a different, more localized delivery route) may be employed to the extent that subject tolerance permits. Multiple doses per day are also contemplated in some cases to achieve appropriate systemic levels of the composition within the subject or within the active site of the subject.

The dose of the composition to the subject may be such that a therapeutically effective amount of the composition reaches the active site of the composition within the subject, i.e., dendritic cells and/or other antigen-presenting cells within the body. The dosage may be given in some cases at the maximum amount while avoiding or minimizing any potentially detrimental side effects within the subject. The dosage of the composition that is actually administered is dependent upon factors such as the final concentration desired at the active site, the method of administration to the subject, the efficacy of the composition, the longevity of the composition within the subject, the timing of administration, the effect of concurrent treatments (e.g., as in a cocktail), etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition at the active site. Variations in dosing may occur between different individuals or even within the same individual on different days. It may be preferred that a maximum dose be used, that is, the highest safe dose according to sound medical judgment. Preferably, the dosage form is such that it does not substantially deleteriously affect the subject. In certain embodiments, the composition may be administered to a subject as a preventive measure. In some embodiments, the inventive composition may be administered to a subject based on demographics or epidemiological studies, or to a subject in a particular field or career.

Administration of a composition of the invention may be accomplished by any medically acceptable method, which allows the composition to reach its target, i.e., dendritic cells and/or other antigen-presenting cells within the body. The particular mode selected will depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the composition within the subject without causing clinically unacceptable adverse effects.

Any medically acceptable method may be used to administer the composition to the subject. The administration may be localized (i.e., to a particular region, physiological system, tissue, organ, or cell type) or systemic, depending on the condition to be treated. For example, the composition may be administered pulmonary, nasally, transdermally, through parenteral injection or implantation, via surgical administration, or any other method of administration where access to the target by the composition of the invention is achieved. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system.

In certain embodiments of the invention, the administration of the composition of the invention may be designed so as to result in sequential exposures to the composition over a certain time period, for example, hours, days, weeks, months or years. This may be accomplished, for example, by repeated administrations of a composition of the invention by one of the methods described above, or by a sustained or controlled release delivery system in which the composition is delivered over a prolonged period without repeated administrations. Administration of the composition using such a delivery system may be, for example, by oral dosage forms, bolus injections, transdermal patches or subcutaneous implants. Maintaining a substantially constant concentration of the composition may be preferred in some cases.

The composition may also be administered on a routine schedule, but alternatively, may be administered as symptoms arise. A “routine schedule” as used herein, refers to a predetermined designated period of time. The routine schedule may encompass periods of time which are identical or which differ in length, as long as the schedule is predetermined. For instance, the routine schedule may involve administration of the composition on a daily basis, every two days, every three days, every four days, every five days, every six days, a weekly basis, a bi-weekly basis, a monthly basis, a bimonthly basis or any set number of days or weeks there-between, every two months, three months, four months, five months, six months, seven months, eight months, nine months, ten months, eleven months, twelve months, etc. Alternatively, the predetermined routine schedule may involve administration of the composition on a daily basis for the first week, followed by a monthly basis for several months, and then every three months after that. Any particular combination would be covered by the routine schedule as long as it is determined ahead of time that the appropriate schedule involves administration on a certain day.

In some cases, the composition is administered to the subject in anticipation of an allergic event in order to prevent an allergic event. The allergic event may be, but need not be limited to, an asthma attack, seasonal allergic rhinitis (e.g., hay-fever, pollen, ragweed hypersensitivity) or perennial allergic rhinitis (e.g., hypersensitivity to allergens such as those described herein). In some instances, the composition is administered substantially prior to an allergic event. As used herein, “substantially prior” means at least six months, at least five months, at least four months, at least three months, at least two months, at least one month, at least three weeks, at least two weeks, at least one week, at least 5 days, or at least 2 days prior to the allergic event.

Similarly, the composition may be administered immediately prior to an allergic event (e.g., within 48 hours, within 24 hours, within 12 hours, within 6 hours, within 4 hours, within 3 hours, within 2 hours, within 1 hour, within 30 minutes or within 10 minutes of an allergic event), substantially simultaneously with the allergic event (e.g., during the time the subject is in contact with the allergen or is experiencing the allergy symptoms) or following the allergic event. In order to desensitize a subject to a particular allergen, the conjugate containing that antigen or allergen may be administered in very small doses over a period of time, consistent with traditional desensitization therapy.

Other delivery systems suitable for use with the present invention include time-release, delayed release, sustained release, or controlled release delivery systems. Such systems may avoid repeated administrations of the composition in many cases, increasing convenience to the subject. Many types of release delivery systems are available and known to those of ordinary skill in the art. They include, for example, polymer-based systems such as polylactic and/or polyglycolic acids, polyanhydrides, polycaprolactones and/or combinations of these; nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the composition. In addition, a pump-based hardware delivery system may be used to deliver one or more embodiments of the invention.

Use of a long-term release device may be particularly suitable in some embodiments of the invention. “Long-term release,” as used herein, means that a device containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above

In certain aspects, the methods and compositions of the present invention can be used for non-therapeutic purposes, such as assays, diagnostics, reagents and kits.

Kits

The invention further provides an article of manufacture and kit containing oligosaccharide materials. The article of manufacture comprises a container with a label. Suitable containers include, for example, bottles, vials, and test tubes. The containers may be formed from a variety of materials such as glass or plastic. The container holds a composition comprising the oligosaccharide preparations described herein. In other embodiments, the kit includes the glycoprotein. The label on the container indicates that the composition is used for the treatment or prevention of a particular disease or disorder, and may also indicate directions for in vivo, such as those described above. The kit of the invention comprises the container described above and a second container comprising a buffer. It may further include other materials desirable from a commercial and user standpoint, including other buffers, diluents, filters, needles, syringes, and package inserts with instructions for use.

Ultimately, synthesis of the various glycoforms in prokaryotes (e.g., E. coli) facilitates attachment to a protein, incorporation into a glycan array, and utilization as a substrate to produce other human-like, N-linked glycans, diagnostics, kits or reagents.

The above disclosure generally describes the present invention. A more specific description is provided below in the following examples. The examples are described solely for the purpose of illustration and are not intended to limit the scope of the present invention. Changes in form and substitution of equivalents are contemplated as circumstances suggest or render expedient. Although specific terms have been employed herein, such terms are intended in a descriptive sense and not for purposes of limitation.

### EXAMPLES

### Example 1

### Plasmid Construction

Vaderrama-Rincon et al. recently disclosed a biosynthetic pathway for the biosynthesis and assembly of Man₃GlcNAc₂ on Und-PP in the cytoplasmic membrane of E. coli. The pathway, which comprises Alg13 Alg14 Alg1 and Alg2 activities with either wild-type nucleotide sequences or codon optimized sequences confers eukaryotic glycosyltransferase activity to the prokaryotic host cell. This pathway serves to add GlcNAc and mannose units to undecaprenol-linked carrier substrate yielding a trimannosyl core oligosaccharide structure. E. coli possesses an integral membrane protein WecA that mediates the transfer of GlcNAc-1-phosphate from UDP-GlcNAc onto undecaprenyl phosphate (Und-P) to form Und-PP-GlcNAc (Rick, P. D. & Silver, R. P. in Escherichia coli and Salmonella: Cellular and Molecular Biology. (ed. F. C. a.o. Neidhardt) 104-122 (American Society for Microbiology, Washingtion, D.C.; 1996). Thus, natively produced Und-PP-GlcNAc exists as a candidate precursor for the desired Man₃GlcNAc₂ glycan. For the addition of the second GlcNAc residue, the Saccharomyces cerevisiae β1,4-GlcNAc transferase that is comprised of two subunits, Alg13 and Alg14 was expressed. In yeast, Alg14 is an integral membrane protein that functions as a membrane anchor to recruit soluble Alg13 to the cytosolic face of the ER, where catalysis to Dol-PP-GlcNAc₂ occurs (Bickel, T. et al., Biosynthesis of lipid-linked oligosaccharides in Saccharomyces cerevisiae: Alg13p and Alg14p form a complex required for the formation of GlcNAc(2)-PP-dolichol. J Biol Chem 280, 34500-34506 (2005)). When co-expressed in E. coli, Alg14 was observed to localize in the membrane fraction while Alg13 was found in both the cytoplasm and membrane fractions, consistent with the situation in yeast. For the subsequent steps, S. cerevisiae β1,4-mannosyltransferase Alg1, which attaches the first mannose to the glycan, and the bifunctional mannosyltransferase Alg2, which catalyzes the addition of both the α1,3- and α1,6-mannose residues to the glycan was expressed (O'Reilly, M. K., et al., In vitro evidence for the dual function of Alg2 and Alg11: essential mannosyltransferases in N-linked glycoprotein biosynthesis. Biochemistry 45, 9593-9603 (2006)). Following expression in E. coli, both Alg1 and Alg2 localized in cell membranes. To determine if the correctly localized Alg enzymes were capable of producing Man₃GlcNAc₂ on Und-PP, a plasmid pYCG (Valderrama-Rincon et al.) that permits simultaneous expression of Alg13, Alg14, Alg1 and Alg2 was constructed.

Plasmid pMQ70 (Shanks et. al., 2006 AEM. 72(7)5027-5036.) was linearized with Ahd1 which is an isoschizomer of Eam11051. The p15a on and cat gene were amplified from pBAD33 and used to co-transform yeast with the linearized vector pMQ70. Homologous recombination in yeast resulted in replacement of the colE1 on and bla gene generating vector pMW07 (Valderrama-Rincon et al.). Table 3 lists the construction and genotype of various strains.

### Example 2

### Analytical Protocols

The method for extraction and purification of the N-linked oligosaccharide was followed as described in Gao et al. (Gao et al., “Non-radioactive analysis of lipid linked oligosaccharide composition by fluorophore-assisted carbohydrate electrophoresis,” Method Enzymol 415: 3-20). The purified oligosaccharides were analyzed by MALDI-TOF mass spectrometry using dihydroxybenzoic acid (DHB) as the matrix (AB Sciex TOF/TOF 5800).

The glycan figures are in standard CFG (Consortium for Functional Genomics) black and white notation, which were generated in GlycoWorkbench 2.0.

### Example 3

### Production of Human-Like N-Linked Man₅GlcNAc₂ High Mannose Oligosaccharide in E. coli

In humans, and other eukaryotes, the Man₅GlcNAc₂ glycoform is a key intermediate in glycan synthesis. In eukaryotes, this key glycoform is synthesized on the cytosolic side of the endoplasmic reticulum membrane. The enzyme Alg11 catalyzes the addition of two, α1,2-mannose residues to the α1,3 mannose of the Man₃GlcNAc₂ glycan core. The gene encoding Alg11 from Saccharomyces cerevisiae was cloned as a fusion to the gene (gst) encoding glutathione S-transferase into plasmid pMW07-YCG-PglB.CO which is used for production of the Man₃GlcNAc₂ trimannosyl core (Valderrama-Rincon et al.) The resulting plasmid was transformed into E. coli MC4100 ΔwaaL gmd::kan by electroporation (Gly02). Gly02 was grown in 100 mL of Luria-Bertani (LB) broth and induced by adding 0.2% (v/v) arabinose once the culture reached an optical density of 3.0.

Analysis of the purified oligosaccharides by mass spectrometry revealed a predominant peak (m/z 1257.6 Na+) consistent with the desired Man₅GlcNAc₂ glycoform (FIG. 1A). In some samples, a minor peak appeared, which was consistent with the Man₃GlcNAc₂ glycoform (m/z 933.5 Na+). In other examples, minor peaks including glycans consistent with Man₂GlcNAc₂, Man₄GlcNAc, Man₃GlcNAc₂, HexMan₃GlcNAc₂, HexMan₅GlcNAc Man₆GlcNAc appeared. To confirm the addition of the expected α1,2 mannose residues to the Man₃GlcNAc₂ glycan core, purified glycans were treated with a α1,2 mannosidase (Prozyme) according to manufacturer's protocol. Following incubation with the enzyme, glycans were labeled and analyzed by mass spectrometry and a FACE gel in the method of Gao et al. In the untreated sample, a predominant peak consistent with the Man₅GlcNAc₂ glycoform was observed (not shown). In the treated sample, a predominant peak (m/z 933.4 Na+) consistent with a Man₃GlcNAc₂ glycoform was observed (FIG. 1B). This confirms the expected addition of two α1,2-mannose residues to the Man₃GlcNAc₂ glycan core. As a result, the human-like Man₅GlcNAc₂ glycoform can be produced by expression of Alg11 in E. coli. Isolation of the Man₅GlcNAc₂ glycoform is challenging by other means since, in eukaryotes, it is a transient oligosaccharide. Synthesis of Man₅GlcNAc₂ in this system was also challenging due to difficulty in expression of a sufficient amount of active enzyme. Various fusion partners, along with Alg11 alone, were explored and resulted in the lack of efficient product formation for majority of the Alg11 moieties examined. Both the GST and MstX fused to Alg11 produced the Man₅GlcNAc₂ glycoform in this system.

### Example 4

### Production of Hybrid N-Linked GlcNAcMan₃GlcNAc₂ Oligosaccharide in E. coli

In humans, and other eukaryotes, the GlcNAcMan₃GlcNAc₂ glycoform is a key intermediate in glycan synthesis. This glycoform is typically only found on N-linked glycans attached to proteins in the Golgi of eukaryotes. Here the glycan was assembled on a lipid carrier in E. coli. To accomplish this, the gene encoding a truncated form (residues 30-446) of Nicotiana tabaccum N-acetylglucosaminyltransferase I (GnTI) was synthesized. The GnTI gene was amplified by PCR and subcloned into the plasmid pMQ70 as a fusion to the gene (malE) encoding E. coli maltose binding protein (MBP) lacking its native signal sequence. The resulting pMQ70-MBP-NtGnTI was transformed into E. coli MC4100 ΔwaaL gmd::kan (Gly03) and Origami2 gmd::kan (Gly03.1) by electroporation along with a second plasmid pMW07-YCG-PglB.CO for production of the Man₃GlcNAc₂ trimannosyl core (Valderrama-Rincon et al.) and grown in 100 mL of Luria-Bertani (LB) broth. Glycosyltransferase expression was induced by adding 0.2% (v/v) arabinose once the culture reached an optical density of 3.0.

Analysis of the purified oligosaccharides by mass spectrometry revealed a predominant peak (m/z 1136.5 Na+) consistent with the desired GlcNAcMan₃GlcNAc₂ glycoform (FIG. 2A). A minor peak was consistent with the Man₃GlcNAc₂ glycoform (m/z 933.4 Na+). To confirm the addition of the expected GlcNAc to the Man₃GlcNAc₂ glycan core, purified glycans were treated with a β-N-acetylglucosaminidase (New England Biolabs) according to manufacturer's protocol. Following incubation with the enzyme, glycans were labeled and analyzed by mass spectrometry, and a FACE gel in the method of Gao et al. In the untreated sample, a predominant peak consistent with the GlcNAcMan₃GlcNAc₂ glycoform was observed (not shown). In the treated sample, the predominant peak is consistent with a Man₃GlcNAc₂ glycoform (FIG. 2B). This confirms the expected addition of a β-GlcNAc to the Man₃GlcNAc₂ glycan core. As a result, the human-like GlcNAcMan₃GlcNAc₂ glycoform can be produced by expression of GnTI in E. coli. Isolation of the GlcNAcMan₃GlcNAc₂ glycoform is challenging by other means since, in eukaryotes, it is a transient oligosaccharide. Obstacles were also encountered using this system, where expression of human GnTI alone, or fused to mstX, in E. coli was first attempted and did not efficiently produce the desired GlcNAcMan₃GlcNAc₂ glycoform (figure not shown). Moreover, when not fused to MBP, the N. tabaccum GnTI failed to produce the desired product (figure not shown).

### Example 5

### Production of N-Linked GlcNAc₂Man₃GlcNAc₂ Complex Oligosaccharide in E. coli

In humans, and other eukaryotes, the GlcNAc₂Man₃GlcNAc₂ complex glycoform (“G0”) is a key intermediate in glycan synthesis, as it is the “core” by which the glycan is fully decorated. This glycoform is typically only found on N-linked glycans attached to proteins in the Golgi of eukaryotes. Here the glycan was assembled on a lipid carrier in E. coli. To accomplish this, the gene encoding a truncated form (residues 30-447) of human N-acetylglucosaminyltransferase II (GnTII) was synthesized. The GnTII gene was amplified by PCR and subcloned into the plasmid pMQ70 as a fusion to MBP lacking its native signal sequence. The resulting pMQ70-MBP-hGnTII was transformed into E. coli MC4100 ΔwaaL gmd::kan, (gly06) Origami2 gmd::kan (Gly06.1), DR473 gmd::kan (gly06.2) and Shuffle ΔwaaL gmd::kan (Gly06.3) by electroporation along with a second plasmid pMW07-YCG-MBP-NtGnTI for production of the GlcNAcMan₃GlcNAc₂ substrate oligosaccharide. Glycosyltransferase expression was induced with 0.2% (v/v) arabinose, added immediately upon inoculation into 1 L of Luria-Bertani (LB) broth.

Analysis of the purified oligosaccharides by mass spectrometry revealed a predominant peak (m/z 1339.8 Na+) consistent with the desired GlcNAc₂Man₃GlcNAc₂ glycoform (FIG. 3). A minor peak was consistent with the Man₃GlcNAc₂ glycoform (m/z 933.5 Na+). A second minor peak consistent with GlcNAcMan₃GlcNAc₂ (m/z 1136.6 Na+) was also observed in the spectrum. Expression of GnTII in the glycoengineered E. coli proved to be challenging, where GnTII from three organisms were examined by expression alone, or when fused to mstX or MBP. Additionally, GnTII expression was examined in both oxidative and non-oxidative bacterial strains. Of the six GnTII moieties and four bacterial strains examined, efficient production of the GlcNAc₂Man₃GlcNAc₂ glycan was seen with MBP-fused, human GnTII in one of the four bacterial strains (figure not shown).

### Example 6

### Production of Branched N-Linked GlcNAc₂Man₃GlcNAc₂ Hybrid Oligosaccharide in E. coli

Synthesis of multiantennary, N-linked glycans is a common feature in humans and other eukaryotes. Production of triantennary oligosaccharides is accomplished by the addition of a GlcNAc residue to GlcNAc₂Man₃GlcNAc₂ by N-acetylglucosaminyltransferase IV (GnTIV). GnTIV can also act on GlcNAcMan₃GlcNAc₂, producing a biantennary, hybrid oligosaccharide that is a structural isomer of the GlcNAc₂Man₃GlcNAc₂ complex glycan. The bacterial codon optimized gene encoding a truncated form (residues 93-535) of bovine GnTIV was synthesized. The GnTIV gene was amplified by PCR and subcloned into the plasmid pMQ70 as a fusion to MBP lacking its native signal sequence. The resulting pMQ70-MBP-hGnTIV was transformed into E. coli MC4100 ΔwaaL gmd::kan (Gly05) and Origami2 gmd::kan (Gly05.1) by electroporation along with a second plasmid pMW07-YCG-MBP-NtGnTI for production of the GlcNAcMan₃GlcNAc₂ substrate oligosaccharide. Glycosyltransferase expression was induced with 0.2% (v/v) arabinose, added immediately upon inoculation into 1 L of Luria-Bertani (LB) broth.

Analysis of the purified oligosaccharides by mass spectrometry revealed a predominant peak (m/z 1339.7 Na+) consistent with the desired GlcNAc₂Man₃GlcNAc₂ glycoform (FIG. 4A). In some samples, a minor peak was consistent with the Man₃GlcNAc₂ glycoform (m/z 933.5 Na+). To confirm the addition of the expected GlcNAc to the GlcNAcMan₃GlcNAc₂ glycan, purified glycans were treated with a β-N-acetylglucosaminidase (New England Biolabs) according to manufacturer's protocol. Following incubation with the enzyme, glycans were labeled and analyzed by mass spectrometry. In the untreated sample, a predominant peak consistent with the GlcNAc₂Man₃GlcNAc₂ glycoform was observed (not shown). In the treated sample, the predominant peak is consistent with a Man₃GlcNAc₂ glycoform (FIG. 4B). This confirms the expected addition of a β-GlcNAc to the GlcNAcMan₃GlcNAc₂ glycan core. Expression of GnTIV in the glycoengineered E. coli proved to be challenging, where GnTIV expression was examined in both oxidative and non-oxidative bacterial strains. Efficient production of the GlcNAc₂Man₃GlcNAc₂ glycan was only seen in the oxidative bacterial strain (figure not shown).

### Example 7

### Production of Multiple Antennary N-Linked GlcNAc₃Man₃GlcNAc₂ Complex Oligosaccharide in E. coli

Synthesis of triantennary, N-linked glycans is a feature found in humans and other eukaryotes. Production of one such triantennary oligosaccharide is accomplished by the addition of a UDP-GlcNAc residue to GlcNAc₂Man₃GlcNAc₂ by N-acetylglucosaminyltransferase IV (GnTIV). The codon optimized gene encoding bovine GnTIV was synthesized. The GnTIV gene was amplified by PCR and subcloned past the 3′-end of the human GnTII gene in the plasmid pMQ70-MBP-hGnTII. The resulting construct was transformed into E. coli cells (Origami2 gmd::kan) by electroporation along with a second plasmid pMW07-YCG-MBP-NtGnTI for production of the GlcNAcMan₃GlcNAc₂ substrate oligosaccharide to create strain GLY06.4. Glycosyltransferase expression was induced with 0.2% (v/v) arabinose, added immediately upon inoculation into 1 L of Luria-Bertani (LB) broth. The method for extraction and purification of the N-linked oligosaccharide was followed as described in Gao et al. The purified oligosaccharides were analyzed by MALDI-TOF mass spectrometry using DHB as the matrix (AB Sciex TOF/TOF 5800).

Analysis of sample glycans from GLY06.4 confirmed a peak consistent with the GlcNAc₃Man₃GlcNAc₂ glycoform (m/z 1543.1 Na+) and also showed a peak consistent with the G0 glycoform (m/z 1339.9 Na+) (FIG. 5B). To confirm the addition of the expected GlcNAc to the G0 glycan, purified glycans were treated with a β-N-acetylglucosaminidase (New England Biolabs) according to manufacturer's protocol. Following incubation with the enzyme, glycans were labeled and analyzed by mass spectrometry.

To generate the substrate oligosaccharide G0(1), a 1 L dense culture of GLY01.5 was induced with 0.2% v/v arabinose for 20 hr at 30° C. The oligosaccharide was isolated by following the methods described in Gao et al. The glycosyltransferases were expressed in a separate, 100 mL culture by induction with 0.2% v/v arabinose for 16 hr at 25° C. This culture was pelleted by centrifugation and resuspended in 2 ml of GnTIV activity buffer (50 mM tris, 10 mM MnCl₂, pH 7.5) and sonicated. The lysate that contained active GnTII was clarified by centrifugation and 20 uL was added to the dried substrate (˜5 μg). An excess of nucleotide-sugar (20 μg) was added to the reaction and subsequently incubated at 30° C. The reaction was monitored by MALDI-TOF mass spectrometry at various time points over a 24 hr period. Once the GnTII reaction was complete, 5 uL of clarified lysate that contained GnTIV was added to the reaction mixture and monitored by MALDI-TOF mass spectrometry at various time points over a 24 hr period.

Analysis of the purified oligosaccharides by mass spectrometry revealed a peak (m/z 1542.9 Na+) consistent with the desired GlcNAc₃Man₃GlcNAc₂ glycoform (FIG. 5A).

### Example 7

### Production of the N-Linked GalGlcNAcMan₃GlcNAc₂ Hybrid Oligosaccharide in E. coli

In humans, and other eukaryotes, GalGlcNAcMan₃GlcNAc₂ glycoform is an intermediate in glycan synthesis. This glycoform is somewhat atypical in healthy adults, but has been seen in individuals with prostate cancer (Kyselova et al., “Alterations in the serum glycome due to metastatic prostate cancer,” J. Proteome Res. (2007)). Here the glycan was assembled on a lipid carrier in E. coli. The gene encoding Helicobacter pylori β-1,4-galactosyltransferase (GalT) was synthesized, amplified by PCR, and subcloned into the plasmid pMQ70. The resulting pMQ70-HpGalT was transformed into MC4100 ΔwaaL gmd::kan (Gly04) and Origami2 gmd::kan (Gly04.1) by electroporation along with a second plasmid pMW07-YCG-MBP-NtGnTI for production of the GlcNAcMan₃GlcNAc₂ substrate oligosaccharide. Glycosyltransferase expression was induced with 0.2% (v/v) arabinose, added immediately upon inoculation into 1 L of Luria-Bertani (LB) broth.

Analysis of sample glycans from GLY04.1 confirmed a predominant speak consistent with the desired GalGlcNAcMan₃GlcNAc₂ glycoform (m/z 1298.7 Na+) (FIG. 6). In some samples, a minor peak was consistent with the Man₃GlcNAc₂ glycoform (m/z 933.5 Na+) (figure not shown). Expression of GalT in the glycoengineered E. coli proved to be challenging, where the GalT from bovine and human, both unfused and fused to MBP and MstX, and Neisseria meningitides did not produce the desired oligosaccharide in E. coli (not shown). Moreover, expression of H. pylori GalT was examined in both oxidative and non-oxidative bacterial strains and efficient galactosylation by was only seen in the oxidative bacterial strain.

### Example 8

### Production of N-Linked Gal₂GlcNAc₂Man₃GlcNAc₂ Complex Oligosaccharide in E. coli

In humans, and other eukaryotes, Gal₂GlcNAc₂Man₃GlcNAc₂ glycoform is a key intermediate in glycan synthesis. This glycoform is typically only found on N-linked glycans attached to proteins in eukaryotes.

The glycans that were assembled on a lipid carrier in E. coli were produced ex vivo using the methods as described in Example 7 with the exception of using GalT rather than GnTIV as the final enzymatic step. Analysis of the purified oligosaccharides by mass spectrometry revealed a predominant peak (m/z 1664.1 Na+) consistent with the desired Gal₂GlcNAc₂Man₃GlcNAc₂ glycoform (FIG. 7A).

For in vivo synthesis of terminally galactosylated glycans, the gene encoding Helicobacter pylori β-1,4-galactosyltransferase (GalT) was synthesized, amplified by PCR, and subcloned into the plasmid pMQ132. The resulting pMQ132-HpGalT was transformed into Origami2 gmd::kan (Gly04.2) by electroporation along with a second plasmid pMW07-YCG-MBP-NtGnTI and a third plasmid pMQ70-MBP-hGnTII for production of the GlcNAc₂Man₃GlcNAc₂ substrate oligosaccharide. Glycosyltransferase expression was induced with 0.2% (v/v) arabinose, added immediately upon inoculation into 1 L of Luria-Bertani (LB) broth.

Analysis of glycans synthesized in Gly04.2 revealed a peak (m/z 1662.2 Na+) consistent with G2 glycoform, a peak (m/z 1500.0 Na+) consistent with the G1 glycoform, and a peak (m/z 1337.9 Na+) consistent with G0 glycoform. The same challenges described in Example 7 were encountered when producing the Gal₂GlcNAc₂Man₃GlcNAc₂ glycoform in E. coli, since the same enzyme was used to produce both products.

### Example 8

### Production of N-Linked NANAGalGlcNAcMan₃GlcNAc₂ Hybrid Oligosaccharide in E. coli

To generate the substrate oligosaccharide GalGlcNAcMan₃GlcNAc₂, a 1 L dense culture of GLY04.1 was induced with 0.2% v/v arabinose for 20 hr at 30° C. The substrate oligosaccharide was isolated by following the methods described in Gao et al. The glycosyltransferases were expressed in a separate, 100 mL culture by induction with 0.2% v/v arabinose for 16 hr at 25° C. This culture was pelleted by centrifugation and resuspended in 2 ml of ST6 activity buffer (50 mM tris, 10 mM MnCl₂, pH 7.5) and sonicated. The lysate was clarified by centrifugation and 20 uL was added to the dried substrate (˜5 μg). An excess of nucleotide-sugar (20 μg) was added to the reaction and subsequently incubated at 30° C. The reaction was monitored by negative mode MALDI-TOF mass spectrometry at various time points over a 24 hr period.

Analysis of the purified oligosaccharides by mass spectrometry revealed a peak (m/z 1565.7 Na+) consistent with the desired NANAGalGlcNAcMan₃GlcNAc₂ glycoform (FIG. 8).

### Example 9

### Optimization of N-Linked Glycan Yield in E. coli

There are a number of advantages to increasing the amount N-linked glycans produced in the glycoengineered E. coli that include: (i) increased glycoprotein production (ii) and facilitating the production of glycoanalytical tools, such as glycan arrays. Therefore, improvement to the yield of the trimannosyl core glycan, the Man₅GlcNAc₂ glycan, and addition of GlcNAc residues to the trimannosyl core were undertaken. Understanding that the nucleotide-sugar pool in E. coli may be limiting, enzymes in the nucleotide-sugar biosynthesis pathway were targeted for overexpression in the glycoengineered E. coli. Specifically phosphomannomutase (ManB), mannose-1-phosphate guanylyltransferase (ManC), and glutamine-fructose-6-phosphate transaminase (GlmS) were investigated, where ManB and ManC are involved in the formation of GDP-Mannose and GlmS is involved in formation of UDP-GlcNAc.

The genes encoding ManB and ManC from E. coli were bicistronically (ManC/ManB) cloned into the plasmid pMQ70 and transformed into E. coli MC4100 ΔwaaL gmd::kan along with pMW07-YCG-PglB.CO for production of the Man₃GlcNAc₂ trimannosyl core (Valderrama-Rincon et al.) by electroporation (Gly01.2). The gene encoding GlmS from E. coli was cloned into the plasmid pTrc99Y (Valderrama-Rincon et al.) and transformed into E. coli MC4100 ΔwaaL gmd::kan along with pMW07-YCG-MBP-NtGnTI by electroporation (Gly01.3). E. coli MC4100 ΔwaaL gmd::kan containing pMW07-YCG-PglB.CO (Gly01) and E. coli MC4100 ΔwaaL gmd::kan containing pMW07-YCG-MBP-NtGnTI (Gly01.1) were used as controls. Gly01 and Gly01.2 were grown in 100 mL of Luria-Bertani (LB) broth and expression was induced with 0.2% (v/v) arabinose at an optical density (O.D.) of 3.0. Gly01.1 and Gly01.3 were grown in 100 mL LB broth and expression was induced with 0.2% (v/v) arabinose and 1 mM IPTG (Gly01.3 only) at an O.D. of 3.0. The method for extraction and purification of the N-linked oligosaccharide was followed as described in Gao et al. The purified oligosaccharides were analyzed by fluorophore-assisted carbohydrate electrophoresis (FACE) using the methods described in Gao et al.

In the case of Gly01.2, a large increase in the production of the trimannosyl core was observed when compared to Gly01 (FIG. 9A left panel). However, difficulty lied within quantifying the difference in yield, since the Gly01 trimannosyl core band was virtually undetectable. Similarly, in the case of Gly01.3, a large increase in glycan yield was observed when compared to Gly01.1 (FIG. 9A right panel). Additionally, a large increase in GlcNAcMan₃GlcNAc₂ was observed when compared to Gly01.1, which was the goal of targeting this enzyme for overexpression. Since there are a number of enzymes involved in nucleotide-sugar biosynthesis, careful consideration was made in determining which enzymes to target for overexpression in the glycoengineered E. coli, where a number of the enzymes may have little effect on glycan yields.

Glycerol provides a carbon source alternative to glucose so as not to effect gene expression from plasmids via promoter repression, as cAMP levels remain high in E. coli with excess glycerol. Use of glycerol appears to increase glycan yield as shown in FIG. 9B. Pyruvate plays a role in recycling GDP to GTP in the Krebs cycle. GTP is a substrate of GDP-mannose pyrophosphorylase that is required for GDP-mannose formation. Increased glycan yield is also shown with the addition of pyruvate FIG. 9C.

Analysis of the purified oligosaccharides by mass spectrometry of host cells with overexpression of ManC/B revealed virtual elimination of the minor peaks as compared to the host cells without ManC/B overexpression. GLY01.2 produced a single predominant peak (m/z 933.5 Na+) consistent with the desired M3 glycoform (FIG. 10D). GLY02.1 produced a single predominant peak (m/z 1257.7 Na+) consistent with the desired M5 glycoform (FIG. 10E). GLY01.5 produced a single predominant peak (m/z 1136.9 Na+) consistent with the desired hybrid GlcNAcMan₃GlcNAc₂ glycoform (FIG. 10F).

### Example 10

### Glycosylated Glucagon Production in E. coli

The glucagon construct consists of glucagon with an N-linked glycosylation site (DQNAT) (SEQ ID NO: 36) followed by a six-histidine tag (SEQ ID NO: 35) at the C-terminus. Glucagon is expressed as a fusion to the C-terminus of MBP after three consecutive C-terminal TEV protease sites in the vector pTrc99Y. The genes encoding for ManC and ManB were also cloned into this vector past the 3′ end of the glucagon coding region. The resulting plasmid was transformed into E. coli cells (Origami2ΔwaaL, gmd::kan) cells by electroporation along with a corresponding glycosyltransferase plasmid. A 100 mL culture of each strain was grown to an optical density at 600 nm of ˜2.0 and induced with 0.2% v/v arabinose for 16 hr followed by induction with 0.1 mM IPTG for 8 hr at 30° C. Cells were harvested by centrifugation and resuspended in lysis buffer (50 mM PO4 buffer, 300 mM NaCl, pH 8.0), sonicated, and spun to remove debris. The clarified cell lysate was loaded onto a pre-equilibrated Ni-NTA spin column (Qiagen) and washed with buffer containing 30 mM imidazole. The fusion protein was eluted with 200 μL of 300 mM imidazole. Eluted protein was subsequently incubated with 1 μg of TEV protease (Sigma Aldrich) at 30° C. Samples were analyzed by mass spectrometry at various time points over a 24 hr period.

Analysis of MALDI-TOF MS of partially purified glucagon appended with a C-terminal glycosylation site was as follows: from strain (FIG. 11A) GLY01.6 consistent with the expected Man₃GlcNAc₂ glycopeptide (m/z 6283), (FIG. 11B) GLY02.2 consistent with the expected GlcNAcMan₅GlcNAc₂ glycopeptide (m/z 6611), (FIG. 11C) GLY01.7 consistent with the expected GlcNAcMan₃GlcNAc₂ glycopeptide (m/z 6488), and (FIG. 11D) GLY04.3 consistent with the expected GalGlcNAcMan₃GlcNAc₂ glycopeptide (m/z 6649). Asterisks indicate background signals present in all samples independent of glycosyltransferases.

### Example 11

### Mannosylated Vaccine Production in E. coli

To glycosylate antigens with mannose terminal glycans, candidate antigens from pathogenic E. coli are mannosylated in vivo as set forth below. For this study we have selected two candidate antigens from extraintestinal pathogenic E. coli (ExPEC) including hypothetical protein c1275 from UPEC strain CFT073 (Lloyd, A. L., D. A. Rasko, and H. L. T. Mobley, Defining Genomic Islands and Uropathogen-Specific Genes in Uropathogenic Escherichia coli. Journal of Bacteriology, 2007. 189(9): p. 3532-3546.), and fimbrial protein ECOK1_{—}3473 (3473) from strain IHE3034 isolated from a patient with neonatal meningitis (Moriel, D. G., et al., Identification of protective and broadly conserved vaccine antigens from the genome of extraintestinal pathogenic Escherichia coli. Proceedings of the National Academy of Sciences, 2010. 107(20): p. 9072-9077.) These antigens were chosen based on a previous vaccination study that found c1275 and 3473 to be (i) unique to pathogenic versus commensal E. coli, (ii) soluble secreted proteins, (iii) protective to varying degrees in a mouse sepsis model and (iv) expressed in ExPEC (Moriel et al.). In addition, c1275 has a native DXNXT sequence that makes even the untagged protein amenable to glycosylation in our system.

Clone Genes with Candidate Antigens.

Successful expression of candidate antigens in preparation for glycosylation studies requires that proteins encode an acceptor asparagine and are expressed in the periplasm. A GlycTag containing four iterations of an N-glycosylation sequon optimized for the bacterial OST PglB is employed. The signal peptide from E. coli disulfide isomerase I (DsbA) is used which directs export via the SRP pathway and performs well in export of ectopic proteins for glycosylation (Fisher, A. C., et al., Production of Secretory and Extracellular N-Linked Glycoproteins in Escherichia coli. Applied and Environmental Microbiology, 2011. 77(3): p. 871-881.) Proteins are expressed from the isopropyl-β-D-thiogalactopyranoside (IPTG)-inducible TRC promoter to provide appropriate expression levels for use in glycosylation studies (Fisher et al.).

The gene sequence encoding 3473 is obtained (Genewiz) and cloned into plasmid pTrcY to include the signal peptide sequence from E. coli DsbA (ssDsbA) and E. coli MBP as an N-terminal translational fusion for periplasmic localization and solubility. A C-terminal GlycTag bearing the glycosylation sites followed by a 6×-His tag (SEQ ID NO: 35) for use in detection and purification is also included. The resulting plasmid is designated pMBP-3473-GT-6H-TrcY (“6H” disclosed as SEQ ID NO: 35). The c1275 antigen was similarly cloned using the same method to generate plasmid pMBP-1275-GT-6H-TrcY (“6H” disclosed as SEQ ID NO: 35).

Modify Antigens with an Asparagine-Linked Mannose-Terminal Glycan.

The paucimannose oligosaccharide structure is present as normal human N-glycans, and it is currently in use in a human therapeutic (Van Patten, S. M., et al., Effect of mannose chain length on targeting of glucocerebrosidase for enzyme replacement therapy of Gaucher disease. Glycobiology, 2007. 17(5): p. 467-478.). Candidate antigens MBP-1275-GT and MBP-3473-GT are individually co-expressed with pMW07-YCG-PglB.CO in glycosylation host strain MC4100 ΔwaaL Δgmd::kan. After inoculation, 10 liters of culture was grown to an approximate optical density at 600 nm of 3.0 and induced with the addition of 0.2% (v/v) arabinose and 1 mM IPTG. Glycoprotein was isolated by ConA affinity chromatography followed by Nickel affinity chromatography, as previously described (Valerrama-Rincon, et. al.). The partially purified samples were analyzed Western blot using an anti-hexahistidine antibody (“hexahistidine” disclosed as SEQ ID NO: 35) and the ConA lectin (FIG. 12).

TABLE 3
Strain and Plasmid List.

| Strain | Plasmid | Plasmid | Plasmid | E. coli | |
|---|---|---|---|---|---|
| name | 1 | 2 | 3 | strain | Product |
| GLY01 | pMW07- | — | — | MC4100 | Man₃GlcNAc₂ |
| | YCG- | | | ΔwaaL | |
| | PglB.CO | | | Δgmd::kan | |
| GLY01.1 | pMW07- | — | — | MC4100 | GlcNAcMan₃GlcNAc₂ |
| | YCG- | | | ΔwaaL | |
| | MBP- | | | Δgmd::kan | |
| | NtGnTI- | | | | |
| | PglB.CO | | | | |
| GLY01.2 | pMW07- | pMQ70- | — | MC4100 | Man₃GlcNAc₂ |
| | YCG- | ManC/B | | ΔwaaL | |
| | PglB.CO | | | Δgmd::kan | |
| GLY01.3 | pMW07- | pTrc99Y- | — | MC4100 | GlcNAcMan₃GlcNAc₂ |
| | YCG- | GlmS | | ΔwaaL | |
| | MBP- | | | Δgmd::kan | |
| | NtGnTI- | | | | |
| | PglB.CO | | | | |
| GLY01.4 | pMW07- | pMQ70- | | Origami2 | GlcNAcMan₃GlcNAc₂ |
| | YCG- | ManC/B | | Δgmd::kan | |
| | PglB.CO | | | | |
| GLY01.5 | pMW07- | pMQ70- | — | Origami2 | GlcNAcMan₃GlcNAc₂ |
| | YCG- | ManC/B | | Δgmd::kan | |
| | MBP- | | | | |
| | NtGnTI- | | | | |
| | PglB.CO | | | | |
| GLY01.6 | pMW07- | pTrc99Y- | — | Origami2 | Man₃GlcNAc₂- |
| | YCG- | MBP- | | Δgmd::kan | Glucagon |
| | PglB.CO | Glucagon- | | ΔwaaL | |
| | | ManC/B | | | |
| GLY01.7 | pMW07- | pTrc99Y- | — | Origami2 | GlcNAcMan₃GlcNAc₂- |
| | YCG- | MBP- | | Δgmd::kan | Glucagon |
| | MBP- | Glucagon- | | ΔwaaL | |
| | NtGnTI- | ManC/B | | ΔnanA | |
| | PglB.CO | | | | |
| GLY02 | pMW07- | — | — | MC4100 | Man₅GlcNAc₂ |
| | YCG- | | | ΔwaaL | |
| | mstX- | | | Δgmd::kan | |
| | Alg11- | | | | |
| | PglB.CO | | | | |
| GLY02.1 | pMW07- | pMQ70- | — | Origami2 | Man₅GlcNAc₂ |
| | YCG- | ManC/B | | Δgmd::kan | |
| | GST- | | | | |
| | Alg11- | | | | |
| | PglB.CO | | | | |
| GLY02.2 | pMW07- | pTrc99Y- | — | Origami2 | Man₅GlcNAc₂- |
| | YCG- | MBP- | | Δgmd::kan | Glucagon |
| | GST- | Glucagon- | | ΔwaaL | |
| | Alg11- | ManC/B | | ΔnanA | |
| | PglB.CO | | | | |
| GLY02.3 | pMW07- | | — | MC4100 | Man₅GlcNAc₂ |
| | YCG- | | | ΔwaaL | |
| | GST- | | | Δgmd::kan | |
| | Alg11- | | | | |
| | PglB.CO | | | | |
| GLY03 | pMW07- | pMQ70- | — | MC4100 | GlcNAcMan₃GlcNAc₂ |
| | YCG- | MBP- | | ΔwaaL | |
| | PglB.CO | NtGnTI | | Δgmd::kan | |
| GLY03.1 | pMW07- | pMQ70- | — | Origami2 | GlcNAcMan₃GlcNAc₂ |
| | YCG- | MBP- | | Δgmd::kan | |
| | PglB.CO | NtGnTI | | | |
| GLY04 | pMW07- | pMQ70- | — | MC4100 | GalGlcNAc- |
| | YCG- | HpGalT | | ΔwaaL | Man₃GlcNAc₂ |
| | MBP- | | | Δgmd::kan | |
| | NtGnTI- | | | | |
| | PglB.CO | | | | |
| GLY04.1 | pMW07- | pMQ70- | — | Origami2 | GalGlcNAc- |
| | YCG- | HpGalT | | Δgmd::kan | Man₃GlcNAc₂ |
| | MBP- | | | | |
| | NtGnTI- | | | | |
| | PglB.CO | | | | |
| GLY04.2 | pMW07- | pMQ70- | pMQ132- | Origami2 | Gal₂GlcNAc₂- |
| | YCG- | MBP- | HpGalT | Δgmd::kan | Man₃GlcNAc₂ |
| | MBP- | hGnTII | | | |
| | NtGnTI- | | | | |
| | PglB.CO | | | | |
| GLY04.3 | pMW07- | pTrc99Y- | — | Origami2 | GalGlcNAc- |
| | YCG- | MBP- | | Δgmd::kan | Man₃GlcNAc₂- |
| | MBP- | Glucagon- | | ΔwaaL | Glucagon |
| | NtGnTI- | ManC/B | | ΔnanA | |
| | HpGalT- | | | | |
| | PglB.CO | | | | |
| GLY05 | pMW07- | pMQ70- | — | MC4100 | GlcNAc₂- |
| | YCG- | MBP- | | ΔwaaL | Man₃GlcNAc₂ |
| | MBP- | bGnTIV | | Δgmd::kan | |
| | NtGnTI- | | | | |
| | PglB.CO | | | | |
| GLY05.1 | pMW07- | pMQ70- | — | Origami2 | GlcNAc₂- |
| | YCG- | MBP- | | Δgmd::kan | Man₃GlcNAc₂ |
| | MBP- | bGnTIV | | | |
| | NtGnTI- | | | | |
| | PglB.CO | | | | |
| GLY06 | pMW07- | pMQ70- | — | MC4100 | GlcNAc₂Man₃GlcNAc₂ |
| | YCG- | MBP- | | ΔwaaL | |
| | MBP- | hGnTII | | Δgmd::kan | |
| | NtGnTI- | | | | |
| | PglB.CO | | | | |
| GLY06.1 | pMW07- | pMQ70- | — | Origami2 | GlcNAc₂Man₃GlcNAc₂ |
| | YCG- | MBP- | | Δgmd::kan | |
| | MBP- | hGnTII | | | |
| | NtGnTI- | | | | |
| | PglB.CO | | | | |
| GLY06.2 | pMW07- | pMQ70- | — | DR473 | GlcNAc₂Man₃GlcNAc₂ |
| | YCG- | MBP- | | Δgmd::kan | |
| | MBP- | hGnTII | | | |
| | NtGnTI- | | | | |
| | PglB.CO | | | | |
| GLY06.3 | pMW07- | pMQ70- | — | Shuffle | GlcNAc₂Man₃GlcNAc₂ |
| | YCG- | MBP- | | ΔwaaL | |
| | MBP- | hGnTII | | Δgmd::kan | |
| | NtGnTI- | | | | |
| | PglB.CO | | | | |
| GLY06.4 | pMW07- | pMQ70- | — | Origami2 | GlcNAc₃Man₃GlcNAc₂ |
| | YCG- | MBP- | | Δgmd::kan | |
| | MBP- | hGnTII- | | | |
| | NtGnTI- | bGnTIV | | | |
| | PglB.CO | | | | |

Although preferred embodiments have been depicted and described in detail herein, it will be apparent to those skilled in the relevant art that various modifications, additions, substitutions, and the like can be made without departing from the spirit of the invention and these are therefore, considered to be within the scope of the present invention as defined the claims which follow.

Informal Sequence Listing

SEQ ID NO: 1 alg13 codon optimized
ATGGGTATCATCGAAGAAAAAGCTCTGTTCGTTACCTGCGGTGC
TACCGTTCCGTTCCCGAAACTGGTTTCTTGCGTTCTGTCTGACGAATTCTGCCA
GGAACTGATCCAGTACGGTTTCGTTCGTCTGATCATCCAGTTCGGTCGTAACT
ACTCTTCTGAATTCGAACACCTGGTTCAGGAACGTGGTGGTCAGCGTGAATCT
CAGAAAATCCCGATCGACCAGTTCGGTTGCGGTGACACCGCTCGTCAGTACG
TTCTGATGAACGGTAAACTGAAAGTTATCGGTTTCGACTTCTCTACCAAAATG
CAGTCTATCATCCGTGACTACTCTGACCTGGTTATCTCTCACGCTGGTACCGG
TTCTATCCTGGACTCTCTGCGTCTGAACAAACCGCTGATCGTTTGCGTTAACG
ACTCTCTGATGGACAACCACCAGCAGCAGATCGCTGACAAATTCGTTGAACT
GGGTTACGTTTGGTCTTGCGCTCCGACCGAAACCGGTCTGATCGCTGGTCTGC
GTGCTTCTCAGACCGAAAAACTGAAACCGTTCCCGGTTTCTCACAACCCGTCT
TTCGAACGTCTGCTGGTTGAAACCATCTACTCTTAA
SEQ ID NO: 2 alg13
MGIIEEKALFVTCGATVPFPKLVSCVLSDEFCQELIQYGFVRLIIQFGRN
YSSEFEHLVQERGGQRESQKIPIDQFGCGDTARQYVLMNGKLKVIGFDFSTKMQ
SIIRDYSDLVISHAGTGSILDSLRLNKPLIVCVNDSLMDNHQQQIADKFVELGYVW
SCAPTETGLIAGLRASQTEKLKPFPVSHNPSFERLLVETIYS*
SEQ ID NO: 3 alg14 codon optimized
ATGAAAACCGCTTACCTGGCTTCTCTGGTTCTGATCGTTTCTACC
GCTTACGTTATCCGTCTGATCGCTATCCTGCCGTTCTTCCACACCCAGGCTGG
TACCGAAAAAGACACCAAAGACGGTGTTAACCTGCTGAAAATCCGTAAATCT
TCTAAAAAACCGCTGAAAATCTTCGTTTTCCTGGGTTCTGGTGGTCACACCGG
TGAAATGATCCGTCTGCTGGAAAACTACCAGGACCTGCTGCTGGGTAAATCT
ATCGTTTACCTGGGTTACTCTGACGAAGCTTCTCGTCAGCGTTTCGCTCACTTC
ATCAAAAAATTCGGTCACTGCAAAGTTAAATACTACGAATTCATGAAAGCTC
GTGAAGTTAAAGCTACCCTGCTGCAGTCTGTTAAAACCATCATCGGTACCCTG
GTTCAGTCTTTCGTTCACGTTGTTCGTATCCGTTTCGCTATGTGCGGTTCTCCG
CACCTGTTCCTGCTGAACGGTCCGGGTACCTGCTGCATCATCTCTTTCTGGCT
GAAAATCATGGAACTGCTGCTGCCGCTGCTGGGTTCTTCTCACATCGTTTACG
TTGAATCTCTGGCTCGTATCAACACCCCGTCTCTGACCGGTAAAATCCTGTAC
TGGGTTGTTGACGAATTCATCGTTCAGTGGCAGGAACTGCGTGACAACTACCT
GCCGCGTTCTAAATGGTTCGGTATCCTGGTTTAA.
SEQ ID NO: 4 alg14
MKTAYLASLVLIVSTAYVIRLIAILPFFHTQAGTEKDTKDGVNLLKIRK
SSKKPLKIFVFLGSGGHTGEMIRLLENYQDLLLGKSIVYLGYSDEASRQRFAHFIK
KFGHCKVKYYEFMKAREVKATLLQSVKTIIGTLVQSFVHVVRIRFAMCGSPHLF
LLNGPGTCCIISFWLKIMELLLPLLGSSHIVYVESLARINTPSLTGKILYWVVDEFI
VQWQELRDNYLPRSKWFGILV*
SEQ ID NO: 5 alg1 codon optimized
ATGTTCCTGGAAATCCCGCGTTGGCTGCTGGCTCTGATCATCCT
GTACCTGTCTATCCCGCTGGTTGTTTACTACGTTATCCCGTACCTGTTCTACGG
TAACAAATCTACCAAAAAACGTATCATCATCTTCGTTCTGGGTGACGTTGGTC
ACTCTCCGCGTATCTGCTACCACGCTATCTCTTTCTCTAAACTGGGTTGGCAG
GTTGAACTGTGCGGTTACGTTGAAGACACCCTGCCGAAAATCATCTCTTCTGA
CCCGAACATCACCGTTCACCACATGTCTAACCTGAAACGTAAAGGTGGTGGT
ACCTCTGTTATCTTCATGGTTAAAAAAGTTCTGTTCCAGGTTCTGTCTATCTTC
AAACTGCTGTGGGAACTGCGTGGTTCTGACTACATCCTGGTTCAGAACCCGCC
GTCTATCCCGATCCTGCCGATCGCTGTTCTGTACAAACTGACCGGTTGCAAAC
TGATCATCGACTGGCACAACCTGGCTTACTCTATCCTGCAGCTGAAATTCAAA
GGTAACTTCTACCACCCGCTGGTTCTGATCTCTTACATGGTTGAAATGATCTT
CTCTAAATTCGCTGACTACAACCTGACCGTTACCGAAGCTATGCGTAAATACC
TGATCCAGTCTTTCCACCTGAACCCGAAACGTTGCGCTGTTCTGTACGACCGT
CCGGCTTCTCAGTTCCAGCCGCTGGCTGGTGACATCTCTCGTCAGAAAGCTCT
GACCACCAAAGCTTTCATCAAAAACTACATCCGTGACGACTTCGACACCGAA
AAAGGTGACAAAATCATCGTTACCTCTACCTCTTTCACCCCGGACGAAGACA
TCGGTATCCTGCTGGGTGCTCTGAAAATCTACGAAAACTCTTACGTTAAATTC
GACTCTTCTCTGCCGAAAATCCTGTGCTTCATCACCGGTAAAGGTCCGCTGAA
AGAAAAATACATGAAACAGGTTGAAGAATACGACTGGAAACGTTGCCAGAT
CGAATTCGTTTGGCTGTCTGCTGAAGACTACCCGAAACTGCTGCAGCTGTGCG
ACTACGGTGTTTCTCTGCACACCTCTTCTTCTGGTCTGGACCTGCCGATGAAA
ATCCTGGACATGTTCGGTTCTGGTCTGCCGGTTATCGCTATGAACTACCCGGT
TCTGGACGAACTGGTTCAGCACAACGTTAACGGTCTGAAATTCGTTGACCGTC
GTGAACTGCACGAATCTCTGATCTTCGCTATGAAAGACGCTGACCTGTACCA
GAAACTGAAAAAAAACGTTACCCAGGAAGCTGAAAACCGTTGGCAGTCTAA
CTGGGAACGTACCATGCGTGACCTGAAACTGATCCACTAA.
SEQ ID NO: 6 alg1
MFLEIPRWLLALIILYLSIPLVVYYVIPYLFYGNKSTKKRIIIFVLGDVGH
SPRICYHAISFSKLGWQVELCGYVEDTLPKIISSDPNITVHHMSNLKRKGGGTSVI
FMVKKVLFQVLSIFKLLWELRGSDYILVQNPPSIPILPIAVLYKLTGCKLIIDWHNL
AYSILQLKFKGNFYHPLVLISYMVEMIFSKFADYNLTVTEAMRKYLIQSFHLNPK
RCAVLYDRPASQFQPLAGDISRQKALTTKAFIKNYIRDDFDTEKGDKIIVTSTSFT
PDEDIGILLGALKIYENSYVKFDSSLPKILCFITGKGPLKEKYMKQVEEYDWKRC
QIEFVWLSAEDYPKLLQLCDYGVSLHTSSSGLDLPMKILDMFGSGLPVIAMNYPV
LDELVQHNVNGLKFVDRRELHESLIFAMKDADLYQKLKKNVTQEAENRWQSN
WERTMRDLKLIH*
SEQ ID NO: 7 alg2 codon optimized
ATGATCGAAAAAGACAAACGTACCATCGCTTTCATCCACCCGG
ACCTGGGTATCGGTGGTGCTGAACGTCTGGTTGTTGACGCTGCTCTGGGTCTG
CAGCAGCAGGGTCACTCTGTTATCATCTACACCTCTCACTGCGACAAATCTCA
CTGCTTCGAAGAAGTTAAAAACGGTCAGCTGAAAGTTGAAGTTTACGGTGAC
TTCCTGCCGACCAACTTCCTGGGTCGTTTCTTCATCGTTTTCGCTACCATCCGT
CAGCTGTACCTGGTTATCCAGCTGATCCTGCAGAAAAAAGTTAACGCTTACC
AGCTGATCATCATCGACCAGCTGTCTACCTGCATCCCGCTGCTGCACATCTTC
TCTTCTGCTACCCTGATGTTCTACTGCCACTTCCCGGACCAGCTGCTGGCTCA
GCGTGCTGGTCTGCTGAAAAAAATCTACCGTCTGCCGTTCGACCTGATCGAAC
AGTTCTCTGTTTCTGCTGCTGACACCGTTGTTGTTAACTCTAACTTCACCAAAA
ACACCTTCCACCAGACCTTCAAATACCTGTCTAACGACCCGGACGTTATCTAC
CCGTGCGTTGACCTGTCTACCATCGAAATCGAAGACATCGACAAAAAATTCT
TCAAAACCGTTTTCAACGAAGGTGACCGTTTCTACCTGTCTATCAACCGTTTC
GAAAAAAAAAAAGACGTTGCTCTGGCTATCAAAGCTTTCGCTCTGTCTGAAG
ACCAGATCAACGACAACGTTAAACTGGTTATCTGCGGTGGTTACGACGAACG
TGTTGCTGAAAACGTTGAATACCTGAAAGAACTGCAGTCTCTGGCTGACGAA
TACGAACTGTCTCACACCACCATCTACTACCAGGAAATCAAACGTGTTTCTGA
CCTGGAATCTTTCAAAACCAACAACTCTAAAATCATCTTCCTGACCTCTATCT
CTTCTTCTCTGAAAGAACTGCTGCTGGAACGTACCGAAATGCTGCTGTACACC
CCGGCTTACGAACACTTCGGTATCGTTCCGCTGGAAGCTATGAAACTGGGTA
AACCGGTTCTGGCTGTTAACAACGGTGGTCCGCTGGAAACCATCAAATCTTA
CGTTGCTGGTGAAAACGAATCTTCTGCTACCGGTTGGCTGAAACCGGCTGTTC
CGATCCAGTGGGCTACCGCTATCGACGAATCTCGTAAAATCCTGCAGAACGG
TTCTGTTAACTTCGAACGTAACGGTCCGCTGCGTGTTAAAAAATACTTCTCTC
GTGAAGCTATGACCCAGTCTTTCGAAGAAAACGTTGAAAAAGTTATCTGGAA
AGAAAAAAAATACTACCCGTGGGAAATCTTCGGTATCTCTTTCTCTAACTTCA
TCCTGCACATGGCTTTCATCAAAATCCTGCCGAACAACCCGTGGCCGTTCCTG
TTCATGGCTACCTTCATGGTTCTGTACTTCAAAAACTACCTGTGGGGTATCTA
CTGGGCTTTCGTTTTCGCTCTGTCTTACCCGTACGAAGAAATCTAA
SEQ ID NO: 8 alg2
MIEKDKRTIAFIHPDLGIGGAERLVVDAALGLQQQGHSVIIYTSHCDKS
HCFEEVKNGQLKVEVYGDFLPTNFLGRFFIVFATIRQLYLVIQLILQKKVNAYQLI
IIDQLSTCIPLLHIFSSATLMFYCHFPDQLLAQRAGLLKKIYRLPFDLIEQFSVSAAD
TVVVNSNFTKNTFHQTFKYLSNDPDVIYPCVDLSTIEIEDIDKKFFKTVFNEGDRF
YLSINRFEKKKDVALAIKAFALSEDQINDNVKLVICGGYDERVAENVEYLKELQS
LADEYELSHTTIYYQEIKRVSDLESFKTNNSKIIFLTSISSSLKELLLERTEMLLYTP
AYEHFGIVPLEAMKLGKPVLAVNNGGPLETIKSYVAGENESSATGWLKPAVPIQ
WATAIDESRKILQNGSVNFERNGPLRVKKYFSREAMTQSFEENVEKVIWKEKKY
YPWEIFGISFSNFILHMAFIKILPNNPWPFLFMATFMVLYFKNYLWGIYWAFVFAL
SYPYEEI*
SEQ ID NO: 9 alg11
ATGGGCAGTGCTTGGACAAACTACAATTTTGAAGAGGTTAAGT
CTCATTTTGGGTTCAAAAAATATGTTGTATCATCTTTAGTACTAGTGTATGGA
CTAATTAAGGTTCTCACGTGGATCTTCCGTCAATGGGTGTATTCCAGCTTGAA
TCCGTTCTCCAAAAAATCTTCATTACTGAACAGAGCAGTTGCCTCCTGTGGTG
AGAAGAATGTGAAAGTTTTTGGTTTTTTTCATCCGTATTGTAATGCTGGTGGT
GGTGGGGAAAAAGTGCTCTGGAAAGCTGTAGATATCACTTTGAGAAAAGATG
CTAAGAACGTTATTGTCATTTATTCAGGGGATTTTGTGAATGGAGAGAATGTT
ACTCCGGAGAATATTCTAAATAATGTGAAAGCGAAGTTCGATTACGACTTGG
ATTCGGATAGAATATTTTTCATTTCATTGAAGCTAAGATACTTGGTGGATTCT
TCAACATGGAAGCATTTCACGTTGATTGGACAAGCAATTGGATCAATGATTCT
CGCATTTGAATCCATTATTCAGTGTCCACCTGATATATGGATTGATACAATGG
GGTACCCTTTCAGCTATCCTATTATTGCTAGGTTTTTGAGGAGAATTCCTATC
GTCACATATACGCATTATCCGATAATGTCAAAAGACATGTTAAATAAGCTGTT
CAAAATGCCCAAGAAGGGTATCAAAGTTTACGGTAAAATATTATACTGGAAA
GTTTTTATGTTAATTTATCAATCCATTGGTTCTAAAATTGATATTGTAATCACA
AACTCAACATGGACAAATAACCACATAAAGCAAATTTGGCAATCCAATACGT
GTAAAATTATATATCCTCCATGCTCTACTGAGAAATTAGTAGATTGGAAGCA
AAAGTTTGGTACTGCAAAGGGTGAGAGATTAAATCAAGCAATTGTGTTGGCA
CAATTTCGTCCTGAGAAACGTCATAAGTTAATCATTGAGTCCTTTGCAACTTT
CTTGAAAAATTTACCGGATTCTGTATCGCCAATTAAATTGATAATGGCGGGGT
CCACTAGATCCAAGCAAGATGAAAATTATGTTAAAAGTTTACAAGACTGGTC
AGAAAATGTATTAAAAATTCCTAAACATTTGATATCATTCGAAAAAAATCTG
CCCTTCGATAAGATTGAAATATTACTAAACAAATCTACTTTCGGTGTTAATGC
CATGTGGAATGAGCACTTTGGAATTGCAGTTGTAGAGTATATGGCTTCCGGTT
TGATCCCCATAGTTCATGCCTCGGCGGGCCCATTGTTAGATATAGTTACTCCA
TGGGATGCCAACGGGAATATCGGAAAAGCTCCACCACAATGGGAGTTACAA
AAGAAATATTTTGCAAAACTCGAAGATGATGGTGAAACTACTGGATTTTTCTT
TAAAGAGCCGAGTGATCCTGATTATAACACAACCAAAGATCCTCTGAGATAC
CCTAATTTGTCCGACCTTTTCTTACAAATTACGAAACTGGACTATGACTGCCT
AAGGGTGATGGGCGCAAGAAACCAGCAGTATTCATTGTATAAATTCTCTGAT
TTGAAGTTTGATAAAGATTGGGAAAACTTTGTACTGAATCCTATTTGTAAATT
ATTAGAAGAGGAGGAAAGGGGCTGA
SEQ ID NO: 10 Alg11 protein
MGSAWTNYNFEEVKSHFGFKKYVVSSLVLVYGLIKVLTWIFRQW
VYSSLNPFSKKSSLLNRAVASCGEKNVKVFGFFHPYCNAGGGGEKVLWKAVDIT
LRKDAKNVIVIYSGDFVNGENVTPENILNNVKAKFDYDLDSDRIFFISLKLRYLVD
SSTWKHFTLIGQAIGSMILAFESIIQCPPDIWIDTMGYPFSYPIIARFLRRIPIVTYTH
YPIMSKDMLNKLFKMPKKGIKVYGKILYWKVFMLIYQSIGSKIDIVITNSTWTNN
HIKQIWQSNTCKIIYPPCSTEKLVDWKQKFGTAKGERLNQAIVLAQFRPEKRHKLI
IESFATFLKNLPDSVSPIKLIMAGSTRSKQDENYVKSLQDWSENVLKIPKHLISFEK
NLPFDKIEILLNKSTFGVNAMWNEHFGIAVVEYMASGLIPIVHASAGPLLDIVTPW
DANGNIGKAPPQWELQKKYFAKLEDDGETTGFFFKEPSDPDYNTTKDPLRYPNL
SDLFLQITKLDYDCLRVMGARNQQYSLYKFSDLKFDKDWENFVLNPICKLLEEE
ERG*
SEQ ID NO: 11 malE(MBP)
AAAATCGAAGAAGGTAAACTGGTAATCTGGATTAACGGCGATA
AAGGCTATAACGGTCTCGCTGAAGTCGGTAAGAAATTCGAGAAAGATACCGG
AATTAAAGTCACCGTTGAGCATCCGGATAAACTGGAAGAGAAATTCCCACAG
GTTGCGGCAACTGGCGATGGCCCTGACATTATCTTCTGGGCACACGACCGCTT
TGGTGGCTACGCTCAATCTGGCCTGTTGGCTGAAATCACCCCGGACAAAGCG
TTCCAGGACAAGCTGTATCCGTTTACCTGGGATGCCGTACGTTACAACGGCA
AGCTGATTGCTTACCCGATCGCTGTTGAAGCGTTATCGCTGATTTATAACAAA
GATCTGCTGCCGAACCCGCCAAAAACCTGGGAAGAGATCCCGGCGCTGGATA
AAGAACTGAAAGCGAAAGGTAAGAGCGCGCTGATGTTCAACCTGCAAGAAC
CGTACTTCACCTGGCCGCTGATTGCTGCTGACGGGGGTTATGCGTTCAAGTAT
GAAAACGGCAAGTACGACATTAAAGACGTGGGCGTGGATAACGCTGGCGCG
AAAGCGGGTCTGACCTTCCTGGTTGACCTGATTAAAAACAAACACATGAATG
CAGACACCGATTACTCCATCGCAGAAGCTGCCTTTAATAAAGGCGAAACAGC
GATGACCATCAACGGCCCGTGGGCATGGTCCAACATCGACACCAGCAAAGTG
AATTATGGTGTAACGGTACTGCCGACCTTCAAGGGTCAACCATCCAAACCGT
TCGTTGGCGTGCTGAGCGCAGGTATTAACGCCGCCAGTCCGAACAAAGAGCT
GGCGAAAGAGTTCCTCGAAAACTATCTGCTGACTGATGAAGGTCTGGAAGCG
GTTAATAAAGACAAACCGCTGGGTGCCGTAGCGCTGAAGTCTTACGAGGAAG
AGTTGGCGAAAGATCCACGTATTGCCGCCACCATGGAAAACGCCCAGAAAG
GTGAAATCATGCCGAACATCCCGCAGATGTCCGCTTTCTGGTATGCCGTGCGT
ACTGCGGTGATCAACGCCGCCAGCGGTCGTCAGACTGTCGATGAAGCCCTGA
AAGACGCGCAGACTCGTATCACCAAGTAA
SEQ ID NO: 12 MalEprotein (MBP)
KIEEGKLVIWINGDKGYNGLAEVGKKFEKDTGIKVTVEHPDKLEE
KFPQVAATGDGPDIIFWAHDRFGGYAQSGLLAEITPDKAFQDKLYPFTWDAVRY
NGKLIAYPIAVEALSLIYNKDLLPNPPKTWEEIPALDKELKAKGKSALMFNLQEP
YFTWPLIAADGGYAFKYENGKYDIKDVGVDNAGAKAGLTFLVDLIKNKHMNA
DTDYSIAEAAFNKGETAMTINGPWAWSNIDTSKVNYGVTVLPTFKGQPSKPFVG
VLSAGINAASPNKELAKEFLENYLLTDEGLEAVNKDKPLGAVALKSYEEELAKD
PRIAATMENAQKGEIMPNIPQMSAFWYAVRTAVINAASGRQTVDEALKDAQTRI
TK*
SEQ ID NO: 13 mstX
ATGTTTTGTACATTTTTTGAAAAACATCACCGGAAGTGGGACAT
ACTGTTAGAAAAAAGCACGGGTGTGATGGAAGCTATGAAAGTGACGAGTGA
GGAAAAGGAACAGCTGAGCACAGCAATCGACCGAATGAATGAAGGACTGGA
CGCGTTTATCCAGCTGTATAATGAATCGGAAATTGATGAACCGCTTATTCAGC
TTGATGATGATACAGCCGAGTTAATGAAGCAGGCCCGAGATATGTACGGCCA
GGAAAAGCTAAATGAGAAATTAAATACAATTATTAAACAGATTTTATCCATC
TCAGTATCTGAAGAAGGAGAAAAAGAA
SEQ ID NO: 14 MstX protein
MFCTFFEKHHRKWDILLEKSTGVMEAMKVTSEEKEQLSTAIDRMNEG
LDAFIQLYNESEIDEPLIQLDDDTAELMKQARDMYGQEKLNEKLNTIIKQILSISVS
EEGEKE*
SEQ ID NO: 15 GnTI (EC2.4.1.101)
GCGACACAGTCAGAATATGCAGATCGCCTTGCTGCTGCAATTG
AAGCAGAAAATCATTGTACAAGCCAGACCAGATTGCTTATTGACCAGATTAG
CCTGCAGCAAGGAAGAATAGTTGCTCTTGAAGAACAAATGAAGCGTCAGGAC
CAGGAGTGCCGACAATTAAGGGCTCTTGTTCAGGATCTTGAAAGTAAGGGCA
TAAAAAAGTTGATCGGAAATGTACAGATGCCAGTGGCTGCTGTAGTTGTTAT
GGCTTGCAATCGGGCTGATTACCTGGAAAAGACTATTAAATCCATCTTAAAA
TACCAAATATCTGTTGCGTCAAAATATCCTCTTTTCATATCCCAGGATGGATC
ACATCCTGATGTCAGGAAGCTTGCTTTGAGCTATGATCAGCTGACGTATATGC
AGCACTTGGATTTTGAACCTGTGCATACTGAAAGACCAGGGGAGCTGATTGC
ATACTACAAAATTGCACGTCATTACAAGTGGGCATTGGATCAGCTGTTTTACA
AGCATAATTTTAGCCGTGTTATCATACTAGAAGATGATATGGAAATTGCCCCT
GATTTTTTTGACTTTTTTGAGGCTGGAGCTACTCTTCTTGACAGAGACAAGTC
GATTATGGCTATTTCTTCTTGGAATGACAATGGACAAATGCAGTTTGTCCAAG
ATCCTTATGCTCTTTACCGCTCAGATTTTTTTCCCGGTCTTGGATGGATGCTTT
CAAAATCTACTTGGGACGAATTATCTCCAAAGTGGCCAAAGGCTTACTGGGA
CGACTGGCTAAGACTCAAAGAGAATCACAGAGGTCGACAATTTATTCGCCCA
GAAGTTTGCAGAACATATAATTTTGGTGAGCATGGTTCTAGTTTGGGGCAGTT
TTTCAAGCAGTATCTTGAGCCAATTAAACTAAATGATGTCCAGGTTGATTGGA
AGTCAATGGACCTTAGTTACCTTTTGGAGGACAATTACGTGAAACACTTTGGT
GACTTGGTTAAAAAGGCTAAGCCCATCCATGGAGCTGATGCTGTCTTGAAAG
CATTTAACATAGATGGTGATGTGCGTATTCAGTACAGAGATCAACTAGACTTT
GAAAATATCGCACGGCAATTTGGCATTTTTGAAGAATGGAAGGATGGTGTAC
CACGTGCAGCATATAAAGGAATAGTAGTTTTCCGGTACCAAACGTCCAGACG
TGTATTCCTTGTTGGCCATGATTCGCTTCAACAACTCGGAATTGAAGATACTT
AA
SEQ ID NO: 16 GnTI protein
ATQSEYADRLAAAIEAENHCTSQTRLLIDQISLQQGRIVALEEQMK
RQDQECRQLRALVQDLESKGIKKLIGNVQMPVAAVVVMACNRADYLEKTIKSIL
KYQISVASKYPLFISQDGSHPDVRKLALSYDQLTYMQHLDFEPVHTERPGELIAY
YKIARHYKWALDQLFYKHNFSRVIILEDDMEIAPDFFDFFEAGATLLDRDKSIMA
ISSWNDNGQMQFVQDPYALYRSDFFPGLGWMLSKSTWDELSPKWPKAYWDDW
LRLKENHRGRQFIRPEVCRTYNFGEHGSSLGQFFKQYLEPIKLNDVQVDWKSMD
LSYLLEDNYVKHFGDLVKKAKPIHGADAVLKAFNIDGDVRIQYRDQLDFENIAR
QFGIFEEWKDGVPRAAYKGIVVFRYQTSRRVFLVGHDSLQQLGIEDT*
SEQ ID NO: 17 GnT II (EC2.4.1.143)
ATGCGCTTTCGTATCTATAAACGTAAAGTGCTGATCCTGACACT
GGTTGTTGCCGCTTGTGGTTTTGTTCTGTGGAGCAGTAATGGTCGTCAGCGTA
AAAATGAAGCCCTGGCACCTCCTCTGCTGGATGCTGAACCGGCACGTGGTGC
TGGCGGTCGTGGTGGTGATCATCCGTCTGTTGCCGTTGGTATTCGTCGTGTGA
GCAATGTTTCGGCTGCCTCTCTGGTCCCGGCTGTTCCTCAACCTGAAGCTGAT
AACCTGACCCTGCGCTATCGCTCTCTGGTGTATCAACTGAACTTCGATCAAAC
TCTGCGTAACGTGGATAAAGCAGGCACATGGGCTCCTCGTGAACTGGTACTG
GTAGTCCAGGTCCATAATCGTCCGGAATATCTGCGTCTGCTGCTGGATTCTCT
GCGCAAAGCTCAAGGCATCGATAATGTCCTGGTCATCTTCTCTCATGATTTCT
GGAGCACGGAGATTAACCAGCTGATTGCCGGCGTGAATTTTTGTCCTGTGCTG
CAGGTGTTTTTTCCGTTTTCTATCCAACTGTATCCGAACGAATTTCCGGGTTCT
GATCCTCGTGATTGTCCTCGTGATCTGCCTAAAAATGCCGCTCTGAAACTGGG
CTGTATTAATGCCGAGTATCCTGATTCTTTTGGCCACTATCGTGAGGCGAAAT
TTTCTCAGACCAAACATCATTGGTGGTGGAAACTGCATTTCGTGTGGGAACGT
GTGAAAATCCTGCGCGACTATGCTGGCCTGATTCTGTTTCTGGAAGAAGATCA
CTATCTGGCTCCGGACTTTTATCATGTGTTCAAAAAAATGTGGAAACTGAAAC
AGCAGGAATGTCCAGAATGTGATGTGCTGTCACTGGGCACCTATAGTGCTTCT
CGCTCCTTCTATGGTATGGCCGACAAAGTGGACGTTAAAACATGGAAATCCA
CCGAGCACAACATGGGTCTGGCACTGACTCGTAATGCCTATCAAAAACTGAT
TGAGTGTACCGACACCTTTTGTACGTATGATGACTATAACTGGGACTGGACCC
TGCAATATCTGACCGTGAGCTGTCTGCCAAAATTTTGGAAAGTTCTGGTGCCT
CAGATTCCTCGTATCTTTCATGCTGGCGACTGTGGTATGCACCATAAAAAAAC
TTGCCGTCCGTCAACACAATCTGCTCAGATCGAGTCGCTGCTGAATAATAACA
AACAGTATATGTTCCCGGAGACTCTGACAATTTCTGAAAAATTCACCGTGGTC
GCCATTTCTCCGCCTCGTAAAAATGGAGGTTGGGGCGATATCCGTGACCATG
AACTGTGTAAAAGCTATCGTCGTCTGCAGTGA
SEQ ID NO: 18 GnT II (EC2.4.1.143)
MRFRIYKRKVLILTLVVAACGFVLWSSNGRQRKNEALAPPLLDAE
PARGAGGRGGDHPSVAVGIRRVSNVSAASLVPAVPQPEADNLTLRYRSLVYQLN
FDQTLRNVDKAGTWAPRELVLVVQVHNRPEYLRLLLDSLRKAQGIDNVLVIFSH
DFWSTEINQLIAGVNFCPVLQVFFPFSIQLYPNEFPGSDPRDCPRDLPKNAALKLG
CINAEYPDSFGHYREAKFSQTKHHWWWKLHFVWERVKILRDYAGLILFLEEDH
YLAPDFYHVFKKMWKLKQQECPECDVLSLGTYSASRSFYGMADKVDVKTWKS
TEHNMGLALTRNAYQKLIECTDTFCTYDDYNWDWTLQYLTVSCLPKFWKVLVP
QIPRIFHAGDCGMHHKKTCRPSTQSAQIESLLNNNKQYMFPETLTISEKFTVVAIS
PPRKNGGWGDIRDHELCKSYRRLQ*
SEQ ID NO: 19 GnTIV (EC2.4.1.145)
TTGAAAGAACTGACGTCCAAAAAGAGCTTGCAAGTCCCGTCCATCTACTATC
ACTTGCCGCACTTGCTGCAAAACGAGGGCTCTTTGCAACCGGCAGTTCAGAT
CGGCAATGGTCGCACCGGCGTGAGCATTGTTATGGGTATCCCGACCGTGAAA
CGTGAAGTGAAAAGCTATCTGATTGAAACGCTGCATAGCCTGATCGATAACC
TGTACCCGGAAGAAAAACTGGACTGCGTGATTGTCGTTTTCATTGGTGAAAC
CGACACGGATTATGTGAATGGCGTTGTTGCCAATCTGGAAAAAGAGTTCAGC
AAAGAGATCAGCAGCGGCCTGGTTGAGATCATTTCTCCGCCGGAGAGCTATT
ACCCGGATCTGACGAACCTGAAAGAAACCTTCGGTGATAGCAAAGAGCGTGT
CCGTTGGCGCACTAAGCAGAACCTGGACTATTGTTTTCTGATGATGTACGCGC
AAGAAAAGGGTACGTATTACATCCAACTGGAGGACGACATTATTGTGAAGCA
AAACTACTTCAACACCATTAAGAACTTCGCGCTGCAGCTGAGCAGCGAAGAG
TGGATGATTCTGGAGTTCAGCCAGCTGGGCTTCATTGGCAAGATGTTTCAGGC
ACCGGACTTGACCCTGATCGTGGAGTTTATCTTTATGTTCTACAAAGAGAAAC
CGATCGATTGGCTGCTGGATCATATCCTGTGGGTCAAGGTCTGCAATCCGGA
AAAAGATGCCAAGCATTGTGACCGCCAGAAAGCGAATCTGCGTATTCGTTTT
CGTCCTAGCCTGTTCCAACACGTGGGTCTGCACAGCTCTCTGACCGGTAAGAT
CCAAAAGCTGACCGACAAAGATTACATGAAACCGCTGCTGCTGAAGATCCAT
GTCAACCCGCCAGCAGAGGTGAGCACCTCGCTGAAAGTCTACCAGGGTCACA
CTCTGGAGAAAACCTATATGGGCGAGGACTTCTTTTGGGCGATTACGCCTGTT
GCGGGTGACTATATCTTGTTTAAGTTTGACAAGCCGGTTAATGTAGAGAGCTA
CTTGTTTCATAGCGGTAACCAGGATCACCCAGGTGACATTCTGCTGAACACCA
CCGTTGAAGTGTTGCCGCTGAAAAGCGAAGGTCTGGATATTTCGAAAGAAAC
GAAGGATAAGCGTCTGGAGGATGGTTACTTCCGTATCGGCAAGTTCGAGAAT
GGCGTGGCTGAAGGTATGGTCGACCCGAGCCTGAACCCGATTTCCGCATTTC
GCCTGTCCGTCATCCAGAATAGCGCGGTTTGGGCTATCCTGAATGAGATTCAC
ATCAAAAAGGTTACGAATTAA
SEQ ID NO: 20 GnTIV (EC2.4.1.145)
ILKELTSKKSLQVPSIYYHLPHLLQNEGSLQPAVQIGNGRTGVSIVM
GIPTVKREVKSYLIETLHSLIDNLYPEEKLDCVIVVFIGETDTDYVNGVVANLEKE
FSKEISSGLVEIISPPESYYPDLTNLKETFGDSKERVRWRTKQNLDYCFLMMYAQ
EKGTYYIQLEDDIIVKQNYFNTIKNFALQLSSEEWMILEFSQLGFIGKMFQAPDLT
LIVEFIFMFYKEKPIDWLLDHILWVKVCNPEKDAKHCDRQKANLRIRFRPSLFQH
VGLHSSLTGKIQKLTDKDYMKPLLLKIHVNPPAEVSTSLKVYQGHTLEKTYMGE
DFFWAITPVAGDYILFKFDKPVNVESYLFHSGNQDHPGDILLNTTVEVLPLKSEG
LDISKETKDKRLEDGYFRIGKFENGVAEGMVDPSLNPISAFRLSVIQNSAVWAILN
EIHIKKVTN*
SEQ ID NO: 21 GalT (EC2.4.1.38)
ATGCGTGTCTTTATTATCAGTCTGAACCAGAAAGTGTGTGACAA
ATTCGGCCTGGTGTTTCGTGATACCACAACCCTGCTGAATAACATCAATGCCA
CCCGCCACAAAGCACAGATTTTTGACGCCGTCTATAGCAAAACGTTCGAAGG
TGGGCTGCATCCACTGGTGAAAAAACATCTGCACCCGTATTTCATTACCCAGA
ACATCAAAGACATGGGCATTACCACCAACCTGATTAGCGGTGTATCCAAATT
CTATTATGCTCTGAAATATCACGCCAAATTCATGAGCCTGGGCGAACTGGGCT
GTTATGCCAGCCATTATAGCCTGTGGGAGAAATGTATTGAGCTGAACGAGGC
CATTTGTATCCTGGAAGATGACATTACGCTGAAAGAAGATTTCAAAGAGGGC
CTGGATTTCCTGGAAAAACACATTCAGGAGCTGGGCTATGTTCGTCTGATGCA
TCTGCTGTATGATGCCTCCGTTAAAAGCGAACCTCTGTCCCATAAAAACCACG
AGATTCAAGAGCGTGTCGGGATCATTAAAGCTTATAGTCACGGTGTTGGCAC
TCAGGGATATGTGATTACTCCGAAAATTGCCAAAGTGTTCAAAAAATGCTCC
CGTAAATGGGTTGTTCCGGTGGATACGATCATGGATGCCACGTTTATTCATGG
GGTGAAAAACCTGGTACTGCAACCGTTTGTGATTGCCGATGATGAGCAAATT
TCCACGATTGTCCGTAAAGAGGAGCCGTATTCCCCTAAAATTGCCCTGATGCG
CGAACTGCACTTCAAATATCTGAAATATTGGCAGTTTGTGTGA
SEQ ID NO: 22 GalT (EC2.4.1.38)
MRVFIISLNQKVCDKFGLVFRDTTTLLNNINATRHKAQIFDAVYSK
TFEGGLHPLVKKHLHPYFITQNIKDMGITTNLISGVSKFYYALKYHAKFMSLGEL
GCYASHYSLWEKCIELNEAICILEDDITLKEDFKEGLDFLEKHIQELGYVRLMHLL
YDASVKSEPLSHKNHEIQERVGIIKAYSHGVGTQGYVITPKIAKVFKKCSRKWVV
PVDTIMDATFIHGVKNLVLQPFVIADDEQISTIVRKEEPYSPKIALMRELHFKYLK
YWQFV*
SEQ ID NO: 23 manB (EC5.4.2.8)
ATGAAAAAATTAACCTGCTTTAAAGCCTATGATATTCGCGGGAAAT
TAGGCGAAGAACTGAATGAAGATATCGCCTGGCGCATTGGTCGCGCCTATGG
CGAATTTCTCAAACCGAAAACCATTGTGTTAGGCGGTGATGTCCGCCTCACCA
GCGAAACCTTAAAACTGGCGCTGGCGAAAGGTTTACAGGATGCGGGCGTTGA
CGTGCTGGATATTGGTATGTCCGGCACCGAAGAGATCTATTTCGCCACGTTCC
ATCTCGGCGTGGATGGCGGCATTGAAGTTACCGCCAGCCATAATCCGATGGA
TTATAACGGCATGAAGCTGGTTCGCGAGGGGGCTCGCCCGATCAGCGGAGAT
ACCGGACTGCGCGACGTCCAGCGTCTGGCTGAAGCCAACGACTTTCCTCCCG
TCGATGAAACCAAACGCGGTCGCTATCAGCAAATCAACCTGCGTGACGCTTA
CGTTGATCACCTGTTCGGTTATATCAATGTCAAAAACCTCACGCCGCTCAAGC
TGGTGATCAACTCCGGGAACGGCGCAGCGGGTCCGGTGGTGGACGCCATTGA
AGCCCGCTTTAAAGCCCTCGGCGCGCCCGTGGAATTAATCAAAGTGCACAAC
ACGCCGGACGGCAATTTCCCCAACGGTATTCCTAACCCACTACTGCCGGAAT
GCCGCGACGACACCCGCAATGCGGTCATCAAACACGGCGCGGATATGGGCAT
TGCTTTTGATGGCGATTTTGACCGCTGTTTCCTGTTTGACGAAAAAGGGCAGT
TTATTGAGGGCTACTACATTGTCGGCCTGTTGGCAGAAGCATTCCTCGAAAAA
AATCCCGGCGCGAAGATCATCCACGATCCACGTCTCTCCTGGAACACCGTTG
ATGTGGTGACTGCCGCAGGTGGCACGCCGGTAATGTCGAAAACCGGACACGC
CTTTATTAAAGAACGTATGCGCAAGGAAGACGCCATCTATGGTGGCGAAATG
AGCGCCCACCATTACTTCCGTGATTTCGCTTACTGCGACAGCGGCATGATCCC
GTGGCTGCTGGTCGCCGAACTGGTGTGCCTGAAAGATAAAACGCTGGGCGAA
CTGGTACGCGACCGGATGGCGGCGTTTCCGGCAAGCGGTGAGATCAACAGCA
AACTGGCGCAACCCGTTGAGGCGATTAACCGCGTGGAACAGCATTTTAGCCG
TGAGGCGCTGGCGGTGGATCGCACCGATGGCATCAGCATGACCTTTGCCGAC
TGGCGCTTTAACCTGCGCACCTCCAATACCGAACCGGTGGTGCGCCTGAATG
TGGAATCGCGCGGTGATGTGCCGCTGATGGAAGCGCGAACGCGAACTCTGCT
GACGTTGCTGAACGAGTAA
SEQ ID NO: 24 manB (EC5.4.2.8)
MKKLTCFKAYDIRGKLGEELNEDIAWRIGRAYGEFLKPKTIVLGG
DVRLTSETLKLALAKGLQDAGVDVLDIGMSGTEEIYFATFHLGVDGGIEVTASH
NPMDYNGMKLVREGARPISGDTGLRDVQRLAEANDFPPVDETKRGRYQQINLR
DAYVDHLFGYINVKNLTPLKLVINSGNGAAGPVVDAIEARFKALGAPVELIKVH
NTPDGNFPNGIPNPLLPECRDDTRNAVIKHGADMGIAFDGDFDRCFLFDEKGQFI
EGYYIVGLLAEAFLEKNPGAKIIHDPRLSWNTVDVVTAAGGTPVMSKTGHAFIKE
RMRKEDAIYGGEMSAHHYFRDFAYCDSGMIPWLLVAELVCLKDKTLGELVRDR
MAAFPASGEINSKLAQPVEAINRVEQHFSREALAVDRTDGISMTFADWRFNLRTS
NTEPVVRLNVESRGDVPLMEARTRTLLTLLNE*
SEQ ID NO: 25 manC (EC2.7.7.13)
ATGGCGCAGTCGAAACTCTATCCAGTTGTGATGGCAGGTGGCTCCGGTAGCC
GCTTATGGCCGCTTTCCCGCGTACTTTATCCCAAGCAGTTTTTATGCCTGAAA
GGCGATCTCACCATGCTGCAAACCACCATCTGCCGCCTGAACGGCGTGGAGT
GCGAAAGCCCGGTGGTGATTTGCAATGAGCAGCACCGCTTTATTGTCGCGGA
ACAGCTGCGTCAACTGAACAAACTTACCGAGAACATTATTCTCGAACCGGCA
GGGCGAAACACGGCACCTGCCATTGCGCTGGCGGCGCTGGCGGCAAAACGTC
ATAGCCCGGAGAGCGACCCGTTAATGCTGGTATTGGCGGCGGATCATGTGAT
TGCCGATGAAGACGCGTTCCGTGCCGCCGTGCGTAATGCCATGCCATATGCC
GAAGCGGGCAAGCTGGTGACCTTCGGCATTGTGCCGGATCTACCAGAAACCG
GTTATGGCTATATTCGTCGCGGTGAAGTGTCTGCGGGTGAGCAGGATATGGT
GGCCTTTGAAGTGGCGCAGTTTGTCGAAAAACCGAATCTGGAAACCGCTCAG
GCCTATGTGGCAAGCGGCGAATATTACTGGAACAGCGGTATGTTCCTGTTCC
GCGCCGGACGCTATCTCGAAGAACTGAAAAAATATCGCCCGGATATCCTCGA
TGCCTGTGAAAAAGCGATGAGCGCCGTCGATCCGGATCTCAATTTTATTCGCG
TGGATGAAGAAGCGTTTCTCGCCTGCCCGGAAGAGTCGGTGGATTACGCGGT
CATGGAACGTACGGCAGATGCTGTTGTGGTGCCGATGGATGCGGGCTGGAGC
GATGTTGGCTCCTGGTCTTCATTATGGGAGATCAGCGCCCACACCGCCGAGG
GCAACGTTTGCCACGGCGATGTGATTAATCACAAAACTGAAAACAGCTATGT
GTATGCTGAATCTGGCCTGGTCACCACCGTCGGGGTGAAAGATCTGGTAGTG
GTGCAGACCAAAGATGCGGTGCTGATTGCCGACCGTAACGCGGTACAGGATG
TGAAAAAAGTGGTCGAGCAGATCAAAGCCGATGGTCGCCATGAGCATCGGGT
GCATCGCGAAGTGTATCGTCCGTGGGGCAAATATGACTCTATCGACGCGGGC
GACCGCTACCAGGTGAAACGCATCACCGTGAAACCGGGCGAGGGCTTGTCGG
TACAGATGCACCATCACCGCGCGGAACACTGGGTGGTTGTCGCGGGAACGGC
AAAAGTCACCATTGATGGTGATATCAAACTGCTTGGTGAAAACGAGTCCATT
TATATTCCGCTGGGGGCGACGCATTGCCTGGAAAACCCGGGGAAAATTCCGC
TCGATTTAATTGAAGTGCGCTCCGGCTCTTATCTCGAAGAGGATGATGTGGTG
CGTTTCGCGGATCGCTACGGACGGGTGTAA
SEQ ID NO: 26 manC (EC2.7.7.13)
MAQSKLYPVVMAGGSGSRLWPLSRVLYPKQFLCLKGDLTMLQTT
ICRLNGVECESPVVICNEQHRFIVAEQLRQLNKLTENIILEPAGRNTAPAIALAALA
AKRHSPESDPLMLVLAADHVIADEDAFRAAVRNAMPYAEAGKLVTFGIVPDLPE
TGYGYIRRGEVSAGEQDMVAFEVAQFVEKPNLETAQAYVASGEYYWNSGMFLF
RAGRYLEELKKYRPDILDACEKAMSAVDPDLNFIRVDEEAFLACPEESVDYAVM
ERTADAVVVPMDAGWSDVGSWSSLWEISAHTAEGNVCHGDVINHKTENSYVY
AESGLVTTVGVKDLVVVQTKDAVLIADRNAVQDVKKVVEQIKADGRHEHRVH
REVYRPWGKYDSIDAGDRYQVKRITVKPGEGLSVQMHHHRAEHWVVVAGTAK
VTIDGDIKLLGENESIYIPLGATHCLENPGKIPLDLIEVRSGSYLEEDDVVRFADRY
GRV*
SEQ ID NO: 27 glmS (EC2.6.1.16)
ATGTGTGGAATTGTTGGCGCGATCGCGCAACGTGATGTAGCAG
AAATCCTTCTTGAAGGTTTACGTCGTCTGGAATACCGCGGATATGACTCTGCC
GGTCTGGCCGTTGTTGATGCAGAAGGTCATATGACCCGCCTGCGTCGCCTCGG
TAAAGTCCAGATGCTGGCACAGGCAGCGGAAGAACATCCTCTGCATGGCGGC
ACTGGTATTGCTCACACTCGCTGGGCGACCCACGGTGAACCTTCAGAAGTGA
ATGCGCATCCGCATGTTTCTGAACACATTGTGGTGGTGCATAACGGCATCATC
GAAAACCATGAACCGCTGCGTGAAGAGCTAAAAGCGCGTGGCTATACCTTCG
TTTCTGAAACCGACACCGAAGTGATTGCCCATCTGGTGAACTGGGAGCTGAA
ACAAGGCGGGACTCTGCGTGAGGCCGTTCTGCGTGCTATCCCGCAGCTGCGT
GGTGCGTACGGTACAGTGATCATGGACTCCCGTCACCCGGATACCCTGCTGG
CGGCACGTTCTGGTAGTCCGCTGGTGATTGGCCTGGGGATGGGCGAAAACTT
TATCGCTTCTGACCAGCTGGCGCTGTTGCCGGTGACCCGTCGCTTTATCTTCCT
TGAAGAGGGCGATATTGCGGAAATCACTCGCCGTTCGGTAAACATCTTCGAT
AAAACTGGCGCGGAAGTAAAACGTCAGGATATCGAATCCAATCTGCAATATG
ACGCGGGCGATAAAGGCATTTACCGTCACTACATGCAGAAAGAGATCTACGA
ACAGCCGAACGCGATCAAAAACACCCTTACCGGACGCATCAGCCACGGTCAG
GTTGATTTAAGCGAGCTGGGACCGAACGCCGACGAACTGCTGTCGAAGGTTG
AGCATATTCAGATCCTCGCCTGTGGTACTTCTTATAACTCCGGTATGGTTTCC
CGCTACTGGTTTGAATCGCTAGCAGGTATTCCGTGCGACGTCGAAATCGCCTC
TGAATTCCGCTATCGCAAATCTGCCGTGCGTCGTAACAGCCTGATGATCACCT
TGTCACAGTCTGGCGAAACCGCGGATACCCTGGCTGGCCTGCGTCTGTCGAA
AGAGCTGGGTTACCTTGGTTCACTGGCAATCTGTAACGTTCCGGGTTCTTCTC
TGGTGCGCGAATCCGATCTGGCGCTAATGACCAACGCGGGTACAGAAATCGG
CGTGGCATCCACTAAAGCATTCACCACTCAGTTAACTGTGCTGTTGATGCTGG
TGGCGAAGCTGTCTCGCCTGAAAGGTCTGGATGCCTCCATTGAACATGACAT
CGTGCATGGTCTGCAGGCGCTGCCGAGCCGTATTGAGCAGATGCTGTCTCAG
GACAAACGCATTGAAGCGCTGGCAGAAGATTTCTCTGACAAACATCACGCGC
TGTTCCTGGGCCGTGGCGATCAGTACCCAATCGCGCTGGAAGGCGCATTGAA
GTTGAAAGAGATCTCTTACATTCACGCTGAAGCCTACGCTGCTGGCGAACTG
AAACACGGTCCGCTGGCGCTAATTGATGCCGATATGCCGGTTATTGTTGTTGC
ACCGAACAACGAATTGCTGGAAAAACTGAAATCCAACATTGAAGAAGTTCGC
GCGCGTGGCGGTCAGTTGTATGTCTTCGCCGATCAGGATGCGGGTTTTGTAAG
TAGCGATAACATGCACATCATCGAGATGCCGCATGTGGAAGAGGTGATTGCA
CCGATCTTCTACACCGTTCCGCTGCAGCTGCTGGCTTACCATGTCGCGCTGAT
CAAAGGCACCGACGTTGACCAGCCGCGTAACCTGGCAAAATCGGTTACGGTT
GAGTAA
SEQ ID NO: 28 glmS (EC2.6.1.16)
MCGIVGAIAQRDVAEILLEGLRRLEYRGYDSAGLAVVDAEGHMT
RLRRLGKVQMLAQAAEEHPLHGGTGIAHTRWATHGEPSEVNAHPHVSEHIVVV
HNGIIENHEPLREELKARGYTFVSETDTEVIAHLVNWELKQGGTLREAVLRAIPQ
LRGAYGTVIMDSRHPDTLLAARSGSPLVIGLGMGENFIASDQLALLPVTRRFIFLE
EGDIAEITRRSVNIFDKTGAEVKRQDIESNLQYDAGDKGIYRHYMQKEIYEQPNAI
KNTLTGRISHGQVDLSELGPNADELLSKVEHIQILACGTSYNSGMVSRYWFESLA
GIPCDVEIASEFRYRKSAVRRNSLMITLSQSGETADTLAGLRLSKELGYLGSLAIC
NVPGSSLVRESDLALMTNAGTEIGVASTKAFTTQLTVLLMLVAKLSRLKGLDASI
EHDIVHGLQALPSRIEQMLSQDKRIEALAEDFSDKHHALFLGRGDQYPIALEGAL
KLKEISYIHAEAYAAGELKHGPLALIDADMPVIVVAPNNELLEKLKSNIEEVRAR
GGQLYVFADQDAGFVSSDNMHIIEMPHVEEVIAPIFYTVPLQLLAYHVALIKGTD
VDQPRNLAKSVTVE*
SEQ ID NO: 29 (EC2.4.99.1) ST6
ATGAAAAAAATCCTGACCGTGCTGTCCATCTTTATCCTGTCTGCCT
GTAATAGCGACAATACCAGCCTGAAAGAGACTGTTAGCAGCAATTCAGCGGA
TGTTGTGGAAACCGAAACTTATCAACTGACGCCGATCGATGCTCCTTCTTCGT
TCCTGAGCCATTCTTGGGAACAGACCTGTGGTACACCAATTCTGAACGAGTCC
GACAAACAGGCCATTTCCTTCGATTTTGTTGCCCCGGAACTGAAACAAGACG
AGAAATATTGCTTCACCTTCAAAGGCATTACCGGTGATCATCGTTATATCACG
AACACCACTCTGACTGTCGTAGCACCGACACTGGAAGTGTATATCGACCATG
CCAGCCTGCCTAGTCTGCAGCAACTGATCCATATTATCCAGGCGAAAGACGA
ATATCCGAGCAACCAGCGTTTTGTGAGCTGGAAACGTGTTACTGTGGATGCC
GACAACGCCAATAAACTGAACATTCACACCTATCCTCTGAAAGGCAATAACA
CCAGCCCTGAGATGGTAGCGGCGATTGATGAGTATGCCCAGAGCAAAAACCG
TCTGAACATTGAGTTCTATACCAATACGGCCCACGTGTTTAATAACCTGCCGC
CAATCATTCAACCTCTGTATAACAACGAGAAAGTGAAAATCAGCCACATTTC
GCTGTATGATGATGGCAGTAGCGAGTATGTTAGCCTGTATCAGTGGAAAGAC
ACCCCGAATAAAATCGAGACTCTGGAGGGTGAAGTTTCTCTGCTGGCCAACT
ATCTGGCCGGTACAAGTCCTGATGCTCCGAAAGGGATGGGTAACCGCTATAA
TTGGCACAAACTGTATGACACCGACTATTATTTTCTGCGCGAGGATTATCTGG
ACGTGGAAGCCAATCTGCATGATCTGCGCGATTATCTGGGTTCTAGCGCCAA
ACAAATGCCGTGGGATGAATTTGCTAAACTGTCCGATTCTCAGCAAACCCTGT
TCCTGGACATCGTTGGCTTTGATAAAGAGCAGCTGCAACAGCAGTATAGCCA
GTCACCGCTGCCGAACTTCATTTTTACTGGCACCACCACATGGGCAGGGGGT
GAGACAAAAGAGTATTATGCTCAACAACAGGTGAACGTCATCAACAATGCCA
TTAACGAAACCTCCCCATATTATCTGGGTAAAGACTATGACCTGTTCTTTAAA
GGCCATCCGGCTGGAGGAGTGATTAATGATATTATCCTGGGCTCCTTTCCTGA
CATGATTAACATTCCGGCGAAAATCTCATTTGAGGTGCTGATGATGACTGATA
TGCTGCCGGATACCGTTGCTGGAATTGCCTCTTCCCTGTATTTCACCATTCCTG
CCGACAAAGTGAACTTCATCGTGTTCACCAGCAGTGATACCATTACAGACCG
TGAAGAAGCGCTGAAATCTCCTCTGGTTCAGGTGATGCTGACACTGGGTATC
GTGAAAGAAAAAGACGTCCTGTTTTGGGCCGACCATAAAGTGAATAGCATGG
AGGTGGCCATCGACGAAGCGTGTACTCGTATTATCGCCAAACGTCAGCCTAC
CGCTTCAGATCTGCGTCTGGTTATCGCCATTATCAAAACGATCACCGATCTGG
AGCGTATTGGAGATGTTGCCGAAAGCATTGCCAAAGTTGCCCTGGAGAGCTT
TTCTAACAAACAGTATAATCTGCTGGTCAGCCTGGAATCTCTGGGTCAACACA
CCGTTCGTATGCTGCATGAAGTGCTGGATGCTTTTGCCCGTATGGATGTGAAA
GCAGCCATTGAAGTCTATCAGGAGGATGACCGTATCGATCAGGAATATGAGA
GCATTGTCCGTCAACTGATGGCCCATATGATGGAAGATCCGTCTAGCATTCCG
AATGTGATGAAAGTGATGTGGGCAGCTCGTAGTATTGAACGTGTGGGTGACC
GCTGCCAGAACATTTGTGAGTATATCATCTATTTCGTAAAAGGCAAAGATGTT
CGCCACACCAAACCGGATGACTTCGGTACTATGCTGGACTGA
SEQ ID NO: 30 (EC2.4.99.1) ST6
MKKILTVLSIFILSACNSDNTSLKETVSSNSADVVETETYQLTPIDAPSS
FLSHSWEQTCGTPILNESDKQAISFDFVAPELKQDEKYCFTFKGITGDHRYITNTT
LTVVAPTLEVYIDHASLPSLQQLIHIIQAKDEYPSNQRFVSWKRVTVDADNANKL
NIHTYPLKGNNTSPEMVAAIDEYAQSKNRLNIEFYTNTAHVFNNLPPIIQPLYNNE
KVKISHISLYDDGSSEYVSLYQWKDTPNKIETLEGEVSLLANYLAGTSPDAPKGM
GNRYNWHKLYDTDYYFLREDYLDVEANLHDLRDYLGSSAKQMPWDEFAKLSD
SQQTLFLDIVGFDKEQLQQQYSQSPLPNFIFTGTTTWAGGETKEYYAQQQVNVIN
NAINETSPYYLGKDYDLFFKGHPAGGVINDIILGSFPDMINIPAKISFEVLMMTDM
LPDTVAGIASSLYFTIPADKVNFIVFTSSDTITDREEALKSPLVQVMLTLGIVKEKD
VLFWADHKVNSMEVAIDEACTRIIAKRQPTASDLRLVIAIIKTITDLERIGDVAESI
AKVALESFSNKQYNLLVSLESLGQHTVRMLHEVLDAFARMDVKAAIEVYQEDD
RIDQEYESIVRQLMAHMMEDPSSIPNVMKVMWAARSIERVGDRCQNICEYIIYFV
KGKDVRHTKPDDFGTMLD*
SEQ ID NO: 31 MBP-GnTI fusion
ATGAAAATCGAAGAAGGTAAACTGGTAATCTGGATTAACGGCGAT
AAAGGCTATAACGGTCTCGCTGAAGTCGGTAAGAAATTCGAGAAAGATACCG
GAATTAAAGTCACCGTTGAGCATCCGGATAAACTGGAAGAGAAATTCCCACA
GGTTGCGGCAACTGGCGATGGCCCTGACATTATCTTCTGGGCACACGACCGC
TTTGGTGGCTACGCTCAATCTGGCCTGTTGGCTGAAATCACCCCGGACAAAGC
GTTCCAGGACAAGCTGTATCCGTTTACCTGGGATGCCGTACGTTACAACGGC
AAGCTGATTGCTTACCCGATCGCTGTTGAAGCGTTATCGCTGATTTATAACAA
AGATCTGCTGCCGAACCCGCCAAAAACCTGGGAAGAGATCCCGGCGCTGGAT
AAAGAACTGAAAGCGAAAGGTAAGAGCGCGCTGATGTTCAACCTGCAAGAA
CCGTACTTCACCTGGCCGCTGATTGCTGCTGACGGGGGTTATGCGTTCAAGTA
TGAAAACGGCAAGTACGACATTAAAGACGTGGGCGTGGATAACGCTGGCGC
GAAAGCGGGTCTGACCTTCCTGGTTGACCTGATTAAAAACAAACACATGAAT
GCAGACACCGATTACTCCATCGCAGAAGCTGCCTTTAATAAAGGCGAAACAG
CGATGACCATCAACGGCCCGTGGGCATGGTCCAACATCGACACCAGCAAAGT
GAATTATGGTGTAACGGTACTGCCGACCTTCAAGGGTCAACCATCCAAACCG
TTCGTTGGCGTGCTGAGCGCAGGTATTAACGCCGCCAGTCCGAACAAAGAGC
TGGCGAAAGAGTTCCTCGAAAACTATCTGCTGACTGATGAAGGTCTGGAAGC
GGTTAATAAAGACAAACCGCTGGGTGCCGTAGCGCTGAAGTCTTACGAGGAA
GAGTTGGCGAAAGATCCACGTATTGCCGCCACCATGGAAAACGCCCAGAAA
GGTGAAATCATGCCGAACATCCCGCAGATGTCCGCTTTCTGGTATGCCGTGCG
TACTGCGGTGATCAACGCCGCCAGCGGTCGTCAGACTGTCGATGAAGCCCTG
AAAGACGCGCAGACTCGTATCACCAAGGCGACACAGTCAGAATATGCAGAT
CGCCTTGCTGCTGCAATTGAAGCAGAAAATCATTGTACAAGCCAGACCAGAT
TGCTTATTGACCAGATTAGCCTGCAGCAAGGAAGAATAGTTGCTCTTGAAGA
ACAAATGAAGCGTCAGGACCAGGAGTGCCGACAATTAAGGGCTCTTGTTCAG
GATCTTGAAAGTAAGGGCATAAAAAAGTTGATCGGAAATGTACAGATGCCAG
TGGCTGCTGTAGTTGTTATGGCTTGCAATCGGGCTGATTACCTGGAAAAGACT
ATTAAATCCATCTTAAAATACCAAATATCTGTTGCGTCAAAATATCCTCTTTT
CATATCCCAGGATGGATCACATCCTGATGTCAGGAAGCTTGCTTTGAGCTATG
ATCAGCTGACGTATATGCAGCACTTGGATTTTGAACCTGTGCATACTGAAAG
ACCAGGGGAGCTGATTGCATACTACAAAATTGCACGTCATTACAAGTGGGCA
TTGGATCAGCTGTTTTACAAGCATAATTTTAGCCGTGTTATCATACTAGAAGA
TGATATGGAAATTGCCCCTGATTTTTTTGACTTTTTTGAGGCTGGAGCTACTCT
TCTTGACAGAGACAAGTCGATTATGGCTATTTCTTCTTGGAATGACAATGGAC
AAATGCAGTTTGTCCAAGATCCTTATGCTCTTTACCGCTCAGATTTTTTTCCCG
GTCTTGGATGGATGCTTTCAAAATCTACTTGGGACGAATTATCTCCAAAGTGG
CCAAAGGCTTACTGGGACGACTGGCTAAGACTCAAAGAGAATCACAGAGGTC
GACAATTTATTCGCCCAGAAGTTTGCAGAACATATAATTTTGGTGAGCATGGT
TCTAGTTTGGGGCAGTTTTTCAAGCAGTATCTTGAGCCAATTAAACTAAATGA
TGTCCAGGTTGATTGGAAGTCAATGGACCTTAGTTACCTTTTGGAGGACAATT
ACGTGAAACACTTTGGTGACTTGGTTAAAAAGGCTAAGCCCATCCATGGAGC
TGATGCTGTCTTGAAAGCATTTAACATAGATGGTGATGTGCGTATTCAGTACA
GAGATCAACTAGACTTTGAAAATATCGCACGGCAATTTGGCATTTTTGAAGA
ATGGAAGGATGGTGTACCACGTGCAGCATATAAAGGAATAGTAGTTTTCCGG
TACCAAACGTCCAGACGTGTATTCCTTGTTGGCCATGATTCGCTTCAACAACT
CGGAATTGAAGATACTTAA
SEQ ID NO: 32 MBP-GnTII fusion
ATGAAAATCGAAGAAGGTAAACTGGTAATCTGGATTAACGGCGAT
AAAGGCTATAACGGTCTCGCTGAAGTCGGTAAGAAATTCGAGAAAGATACCG
GAATTAAAGTCACCGTTGAGCATCCGGATAAACTGGAAGAGAAATTCCCACA
GGTTGCGGCAACTGGCGATGGCCCTGACATTATCTTCTGGGCACACGACCGC
TTTGGTGGCTACGCTCAATCTGGCCTGTTGGCTGAAATCACCCCGGACAAAGC
GTTCCAGGACAAGCTGTATCCGTTTACCTGGGATGCCGTACGTTACAACGGC
AAGCTGATTGCTTACCCGATCGCTGTTGAAGCGTTATCGCTGATTTATAACAA
AGATCTGCTGCCGAACCCGCCAAAAACCTGGGAAGAGATCCCGGCGCTGGAT
AAAGAACTGAAAGCGAAAGGTAAGAGCGCGCTGATGTTCAACCTGCAAGAA
CCGTACTTCACCTGGCCGCTGATTGCTGCTGACGGGGGTTATGCGTTCAAGTA
TGAAAACGGCAAGTACGACATTAAAGACGTGGGCGTGGATAACGCTGGCGC
GAAAGCGGGTCTGACCTTCCTGGTTGACCTGATTAAAAACAAACACATGAAT
GCAGACACCGATTACTCCATCGCAGAAGCTGCCTTTAATAAAGGCGAAACAG
CGATGACCATCAACGGCCCGTGGGCATGGTCCAACATCGACACCAGCAAAGT
GAATTATGGTGTAACGGTACTGCCGACCTTCAAGGGTCAACCATCCAAACCG
TTCGTTGGCGTGCTGAGCGCAGGTATTAACGCCGCCAGTCCGAACAAAGAGC
TGGCGAAAGAGTTCCTCGAAAACTATCTGCTGACTGATGAAGGTCTGGAAGC
GGTTAATAAAGACAAACCGCTGGGTGCCGTAGCGCTGAAGTCTTACGAGGAA
GAGTTGGCGAAAGATCCACGTATTGCCGCCACCATGGAAAACGCCCAGAAA
GGTGAAATCATGCCGAACATCCCGCAGATGTCCGCTTTCTGGTATGCCGTGCG
TACTGCGGTGATCAACGCCGCCAGCGGTCGTCAGACTGTCGATGAAGCCCTG
AAAGACGCGCAGACTCGTATCACCAAGCGTCAGCGTAAAAATGAAGCCCTG
GCACCTCCTCTGCTGGATGCTGAACCGGCACGTGGTGCTGGCGGTCGTGGTG
GTGATCATCCGTCTGTTGCCGTTGGTATTCGTCGTGTGAGCAATGTTTCGGCT
GCCTCTCTGGTCCCGGCTGTTCCTCAACCTGAAGCTGATAACCTGACCCTGCG
CTATCGCTCTCTGGTGTATCAACTGAACTTCGATCAAACTCTGCGTAACGTGG
ATAAAGCAGGCACATGGGCTCCTCGTGAACTGGTACTGGTAGTCCAGGTCCA
TAATCGTCCGGAATATCTGCGTCTGCTGCTGGATTCTCTGCGCAAAGCTCAAG
GCATCGATAATGTCCTGGTCATCTTCTCTCATGATTTCTGGAGCACGGAGATT
AACCAGCTGATTGCCGGCGTGAATTTTTGTCCTGTGCTGCAGGTGTTTTTTCC
GTTTTCTATCCAACTGTATCCGAACGAATTTCCGGGTTCTGATCCTCGTGATT
GTCCTCGTGATCTGCCTAAAAATGCCGCTCTGAAACTGGGCTGTATTAATGCC
GAGTATCCTGATTCTTTTGGCCACTATCGTGAGGCGAAATTTTCTCAGACCAA
ACATCATTGGTGGTGGAAACTGCATTTCGTGTGGGAACGTGTGAAAATCCTG
CGCGACTATGCTGGCCTGATTCTGTTTCTGGAAGAAGATCACTATCTGGCTCC
GGACTTTTATCATGTGTTCAAAAAAATGTGGAAACTGAAACAGCAGGAATGT
CCAGAATGTGATGTGCTGTCACTGGGCACCTATAGTGCTTCTCGCTCCTTCTA
TGGTATGGCCGACAAAGTGGACGTTAAAACATGGAAATCCACCGAGCACAAC
ATGGGTCTGGCACTGACTCGTAATGCCTATCAAAAACTGATTGAGTGTACCG
ACACCTTTTGTACGTATGATGACTATAACTGGGACTGGACCCTGCAATATCTG
ACCGTGAGCTGTCTGCCAAAATTTTGGAAAGTTCTGGTGCCTCAGATTCCTCG
TATCTTTCATGCTGGCGACTGTGGTATGCACCATAAAAAAACTTGCCGTCCGT
CAACACAATCTGCTCAGATCGAGTCGCTGCTGAATAATAACAAACAGTATAT
GTTCCCGGAGACTCTGACAATTTCTGAAAAATTCACCGTGGTCGCCATTTCTC
CGCCTCGTAAAAATGGAGGTTGGGGCGATATCCGTGACCATGAACTGTGTAA
AAGCTATCGTCGTCTGCAGTGA
SEQ ID NO: 33 MBP-GnTIV fusion
ATGAAAATCGAAGAAGGTAAACTGGTAATCTGGATTAACGGCGAT
AAAGGCTATAACGGTCTCGCTGAAGTCGGTAAGAAATTCGAGAAAGATACCG
GAATTAAAGTCACCGTTGAGCATCCGGATAAACTGGAAGAGAAATTCCCACA
GGTTGCGGCAACTGGCGATGGCCCTGACATTATCTTCTGGGCACACGACCGC
TTTGGTGGCTACGCTCAATCTGGCCTGTTGGCTGAAATCACCCCGGACAAAGC
GTTCCAGGACAAGCTGTATCCGTTTACCTGGGATGCCGTACGTTACAACGGC
AAGCTGATTGCTTACCCGATCGCTGTTGAAGCGTTATCGCTGATTTATAACAA
AGATCTGCTGCCGAACCCGCCAAAAACCTGGGAAGAGATCCCGGCGCTGGAT
AAAGAACTGAAAGCGAAAGGTAAGAGCGCGCTGATGTTCAACCTGCAAGAA
CCGTACTTCACCTGGCCGCTGATTGCTGCTGACGGGGGTTATGCGTTCAAGTA
TGAAAACGGCAAGTACGACATTAAAGACGTGGGCGTGGATAACGCTGGCGC
GAAAGCGGGTCTGACCTTCCTGGTTGACCTGATTAAAAACAAACACATGAAT
GCAGACACCGATTACTCCATCGCAGAAGCTGCCTTTAATAAAGGCGAAACAG
CGATGACCATCAACGGCCCGTGGGCATGGTCCAACATCGACACCAGCAAAGT
GAATTATGGTGTAACGGTACTGCCGACCTTCAAGGGTCAACCATCCAAACCG
TTCGTTGGCGTGCTGAGCGCAGGTATTAACGCCGCCAGTCCGAACAAAGAGC
TGGCGAAAGAGTTCCTCGAAAACTATCTGCTGACTGATGAAGGTCTGGAAGC
GGTTAATAAAGACAAACCGCTGGGTGCCGTAGCGCTGAAGTCTTACGAGGAA
GAGTTGGCGAAAGATCCACGTATTGCCGCCACCATGGAAAACGCCCAGAAA
GGTGAAATCATGCCGAACATCCCGCAGATGTCCGCTTTCTGGTATGCCGTGCG
TACTGCGGTGATCAACGCCGCCAGCGGTCGTCAGACTGTCGATGAAGCCCTG
AAAGACGCGCAGACTCGTATCACCAAGATTTTGAAAGAACTGACGTCCAAAA
AGAGCTTGCAAGTCCCGTCCATCTACTATCACTTGCCGCACTTGCTGCAAAAC
GAGGGCTCTTTGCAACCGGCAGTTCAGATCGGCAATGGTCGCACCGGCGTGA
GCATTGTTATGGGTATCCCGACCGTGAAACGTGAAGTGAAAAGCTATCTGAT
TGAAACGCTGCATAGCCTGATCGATAACCTGTACCCGGAAGAAAAACTGGAC
TGCGTGATTGTCGTTTTCATTGGTGAAACCGACACGGATTATGTGAATGGCGT
TGTTGCCAATCTGGAAAAAGAGTTCAGCAAAGAGATCAGCAGCGGCCTGGTT
GAGATCATTTCTCCGCCGGAGAGCTATTACCCGGATCTGACGAACCTGAAAG
AAACCTTCGGTGATAGCAAAGAGCGTGTCCGTTGGCGCACTAAGCAGAACCT
GGACTATTGTTTTCTGATGATGTACGCGCAAGAAAAGGGTACGTATTACATCC
AACTGGAGGACGACATTATTGTGAAGCAAAACTACTTCAACACCATTAAGAA
CTTCGCGCTGCAGCTGAGCAGCGAAGAGTGGATGATTCTGGAGTTCAGCCAG
CTGGGCTTCATTGGCAAGATGTTTCAGGCACCGGACTTGACCCTGATCGTGGA
GTTTATCTTTATGTTCTACAAAGAGAAACCGATCGATTGGCTGCTGGATCATA
TCCTGTGGGTCAAGGTCTGCAATCCGGAAAAAGATGCCAAGCATTGTGACCG
CCAGAAAGCGAATCTGCGTATTCGTTTTCGTCCTAGCCTGTTCCAACACGTGG
GTCTGCACAGCTCTCTGACCGGTAAGATCCAAAAGCTGACCGACAAAGATTA
CATGAAACCGCTGCTGCTGAAGATCCATGTCAACCCGCCAGCAGAGGTGAGC
ACCTCGCTGAAAGTCTACCAGGGTCACACTCTGGAGAAAACCTATATGGGCG
AGGACTTCTTTTGGGCGATTACGCCTGTTGCGGGTGACTATATCTTGTTTAAG
TTTGACAAGCCGGTTAATGTAGAGAGCTACTTGTTTCATAGCGGTAACCAGG
ATCACCCAGGTGACATTCTGCTGAACACCACCGTTGAAGTGTTGCCGCTGAA
AAGCGAAGGTCTGGATATTTCGAAAGAAACGAAGGATAAGCGTCTGGAGGA
TGGTTACTTCCGTATCGGCAAGTTCGAGAATGGCGTGGCTGAAGGTATGGTC
GACCCGAGCCTGAACCCGATTTCCGCATTTCGCCTGTCCGTCATCCAGAATAG
CGCGGTTTGGGCTATCCTGAATGAGATTCACATCAAAAAGGTTACGAATTAA
SEQ ID NO: 34 GST-alg11 fusion
ATGAAATTGTTCTACAAACCGGGTGCCTGCTCTCTCGCTTCCCATAT
CACCCTGCGTGAGAGCGGAAAGGATTTTACCCTCGTCAGTGTGGATTTAATG
AAAAAACGTCTCGAAAACGGTGACGATTACTTTGCCGTTAACCCTAAGGGGC
AGGTGCCTGCATTGCTGCTGGATGACGGTACTTTGCTGACGGAAGGCGTAGC
GATTATGCAGTATCTTGCCGACAGCGTCCCCGACCGCCAGTTGCTGGCACCG
GTAAACAGTATTTCCCGCTATAAAACCATCGAATGGCTGAATTACATCGCCA
CCGAGCTGCATAAAGGTTTCACACCTCTGTTTCGCCCTGATACACCGGAAGA
GTACAAACCGACAGTTCGCGCGCAGCTGGAGAAGAAGCTGCAATATGTGAAC
GAGGCACTGAAGGATGAGCACTGGATCTGCGGGCAAAGATTTACAATTGCTG
ATGCCTATCTGTTTACGGTTCTGCGCTGGGCATACGCGGTGAAACTGAATCTG
GAAGGGTTAGAGCACATTGCAGCATTTATGCAACGTATGGCTGAACGTCCGG
AAGTACAAGACGCGCTGTCAGCGGAAGGCTTAAAGGGCAGTGCTTGGACAA
ACTACAATTTTGAAGAGGTTAAGTCTCATTTTGGGTTCAAAAAATATGTTGTA
TCATCTTTAGTACTAGTGTATGGACTAATTAAGGTTCTCACGTGGATCTTCCG
TCAATGGGTGTATTCCAGCTTGAATCCGTTCTCCAAAAAATCTTCATTACTGA
ACAGAGCAGTTGCCTCCTGTGGTGAGAAGAATGTGAAAGTTTTTGGTTTTTTT
CATCCGTATTGTAATGCTGGTGGTGGTGGGGAAAAAGTGCTCTGGAAAGCTG
TAGATATCACTTTGAGAAAAGATGCTAAGAACGTTATTGTCATTTATTCAGGG
GATTTTGTGAATGGAGAGAATGTTACTCCGGAGAATATTCTAAATAATGTGA
AAGCGAAGTTCGATTACGACTTGGATTCGGATAGAATATTTTTCATTTCATTG
AAGCTAAGATACTTGGTGGATTCTTCAACATGGAAGCATTTCACGTTGATTGG
ACAAGCAATTGGATCAATGATTCTCGCATTTGAATCCATTATTCAGTGTCCAC
CTGATATATGGATTGATACAATGGGGTACCCTTTCAGCTATCCTATTATTGCT
AGGTTTTTGAGGAGAATTCCTATCGTCACATATACGCATTATCCGATAATGTC
AAAAGACATGTTAAATAAGCTGTTCAAAATGCCCAAGAAGGGTATCAAAGTT
TACGGTAAAATATTATACTGGAAAGTTTTTATGTTAATTTATCAATCCATTGG
TTCTAAAATTGATATTGTAATCACAAACTCAACATGGACAAATAACCACATA
AAGCAAATTTGGCAATCCAATACGTGTAAAATTATATATCCTCCATGCTCTAC
TGAGAAATTAGTAGATTGGAAGCAAAAGTTTGGTACTGCAAAGGGTGAGAG
ATTAAATCAAGCAATTGTGTTGGCACAATTTCGTCCTGAGAAACGTCATAAGT
TAATCATTGAGTCCTTTGCAACTTTCTTGAAAAATTTACCGGATTCTGTATCG
CCAATTAAATTGATAATGGCGGGGTCCACTAGATCCAAGCAAGATGAAAATT
ATGTTAAAAGTTTACAAGACTGGTCAGAAAATGTATTAAAAATTCCTAAACA
TTTGATATCATTCGAAAAAAATCTGCCCTTCGATAAGATTGAAATATTACTAA
ACAAATCTACTTTCGGTGTTAATGCCATGTGGAATGAGCACTTTGGAATTGCA
GTTGTAGAGTATATGGCTTCCGGTTTGATCCCCATAGTTCATGCCTCGGCGGG
CCCATTGTTAGATATAGTTACTCCATGGGATGCCAACGGGAATATCGGAAAA
GCTCCACCACAATGGGAGTTACAAAAGAAATATTTTGCAAAACTCGAAGATG
ATGGTGAAACTACTGGATTTTTCTTTAAAGAGCCGAGTGATCCTGATTATAAC
ACAACCAAAGATCCTCTGAGATACCCTAATTTGTCCGACCTTTTCTTACAAAT
TACGAAACTGGACTATGACTGCCTAAGGGTGATGGGCGCAAGAAACCAGCA
GTATTCATTGTATAAATTCTCTGATTTGAAGTTTGATAAAGATTGGGAAAACT
TTGTACTGAATCCTATTTGTAAATTATTAGAAGAGGAGGAAAGGGGCTGA

What is claimed is:

1. A recombinant prokaryotic host cell that produces an oligosaccharide composition having a terminal mannose residue comprising: one or more N-acetylglucosaminyl transferase enzyme activity (EC 2.4.1.101, EC 2.4.1.143, EC 2.4.1.145, EC 2.4.1.155, EC 2.4.1.201) that catalyzes the transfer of a UDP-GlcNAc residue onto the terminal mannose residue, wherein the host cell produces an oligosaccharide composition having a terminal GlcNAc residue.

2. The host cell of claim 1, wherein the host cell further comprises one or more galactosyltransferase enzyme activity (EC 2.4.1.38) that catalyzes the transfer of a UDP-Galactose residue onto the terminal GlcNAc residue, wherein the host cell produces an oligosaccharide composition having a terminal galactose residue.

3. The host cell of claim 2, wherein the host cell further comprises one or more sialyltransferase enzyme activity SialylT (EC 2.4.99.4, EC 2.4.99.1) that catalyzes the transfer of a CMP-NANA residue onto the terminal galactose residue, wherein the host cell produces an oligosaccharide composition having a terminal sialic acid residue.

4. The host cell of claim 1, 2 or 3, wherein the host cell comprises one or more eukaryotic UDP-GlcNAc transferase enzyme activity (EC 2.4.1.141, EC 2.4.1.145) and one or more eukaryotic mannosyltransferase enzyme activity (EC 2.4.1.142, EC 2.4.1.132).

5. The host cell of claim 1, 2 or 3, wherein the host cell further comprises a fusion of one or more of the enzymes wherein the fusion comprises at least one of the following: DsbA, GlpE, GST, MBP, MstX, NusA and TrxA.

6. The host cell of claim 1, 2 or 3, wherein the host cell is an oxidative host.

7. The host cell of claim 1, 2 or 3, wherein the host cell comprises one or more of the following enzymes: phosphomannomutase enzyme activity (ManB) (EC 5.4.2.8), mannose-1-phosphate guanylyltransferase enzyme activity (ManC) (EC 2.7.7.13) and glutamine-fructose-6-phosphate transaminase enzyme activity (GlmS) (EC 2.6.1.16), wherein the ManB and ManC catalyze GDP-Mannose synthesis and wherein the GlmS catalyzes UDP-GlcNAc synthesis.

8. The host cell of claim 1, 2 or 3, wherein the host cell further comprises an attenuation in GDP-D-mannose dehydratase enzyme activity (EC 4.2.1.47).

9. The host cell of claim 1, 2 or 3, wherein the host cell further comprises a flippase enzyme activity.

10. The host cell of claim 1, 2 or 3, wherein the host cell further comprises an oligosaccharyl transferase enzyme activity (EC 2.4.1.119).

11. The host cell of claim 1, 2 or 3, wherein the host cell further comprises a gene encoding a protein of interest, whereby the host cell produces a glycosylated protein.

12. The host cell of claim 1, 2 or 3, wherein the host cell produces an oligosaccharide composition that is N-linked to a protein.

13. The host cell of claim 1, 2 or 3, wherein the host cell produces a glycosylated protein comprising at least one of the following: an antibody, Fv portion which binds to a native antigen and an Fc portion which is glycosylated at a conserved asparagine residue, diabody, scFv, scFv-Fc, scFv-CH, Fab and scFab.

14. The host cell of claim 1, 2 or 3, wherein the host cell produces a glycosylated protein selected from the following: cytokines such as interferons, G-CSF, coagulation factors such as factor VIII, factor IX, and human protein C, soluble IgE receptor α-chain, IgG, IgG fragments, IgM, interleukins, urokinase, chymase, and urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II, α-feto proteins, AAT, rhTBP-1 (aka TNF binding protein 1), TACI-Ig (transmembrane activator and calcium modulator and cyclophilin ligand interactor), FSH (follicle stimulating hormone), GM-CSF, glucagon, glucagon peptides, GLP-1 w/ and w/o FC (glucagon like protein 1) IL-1 receptor agonist, sTNFr (aka soluble TNF receptor Fc fusion), CTLA4-Ig (Cytotoxic T Lymphocyte associated Antigen 4-Ig), receptors, hormones such as human growth hormone, erythropoietin, peptides, stapled peptides, human vaccines, animal vaccines, serum albumin and enzymes such as ATIII, rhThrombin, glucocerebrosidase and asparaginase.

15. The host cell of claim 1, wherein the host cell expresses a mannosyltransferase, N-acetylglucosaminyl transferase, galactosyltransferase or sialyltransferase operably fused to MBP.

16. The host cell of claim 1, wherein the host cell comprises an oxidative host cell capable of expressing the galactosyltransferase enzyme activity.

17. The host cell of claim 1, wherein the host cell produces oligosaccharide compositions comprising GlcNAc₁₋₅Man₃GlcNAc₂ and Man₃GlcNAc₂.

18. The host cell of claim 1, wherein the oligosaccharide composition is predominantly GlcNAcMan₃GlcNAc₂ or GlcNAc₂Man₃GlcNAc₂.

19. The host cell of claim 2, wherein the host cell produces oligosaccharide compositions comprising Gal₁₋₅GlcNAc₁₋₅Man₃GlcNAc₂ and Man₃GlcNAc₂.

20. The host cell of claim 2, wherein the oligosaccharide composition comprises predominantly GalGlcNAcMan₃GlcNAc₂, GalGlcNAc₂Man₃GlcNAc₂ or Gal₂GlcNAc₂Man₃GlcNAc₂.

21. The host cell of claim 3, wherein the oligosaccharide composition comprises NANA₁₋₅Gal₁₋₅GlcNAc₁₋₅Man₃GlcNAc₂.

22. The host cell of claim 3, wherein the oligosaccharide composition comprises predominantly NANAGalGlcNAcMan₃GlcNAc₂ or NANA₂Gal₂GlcNAc₂Man₃GlcNAc₂.


SEQUENCE LISTING (36 sequences)

SEQ ID NO: 1 - DNA, 609 bp, Artificial Sequence
  Description of Artificial Sequence Synthetic polynucleotide
atgggtatcatcgaagaaaaagctctgttcgttacctgcggtgctaccgttccgttcccgaaactggtttcttgcgttctgtctgacgaattctgccaggaactgatccagtacggtttcgttcgtctgatcatccagttcggtcgtaactactcttctgaattcgaacacctggttcaggaacgtggtggtcagcgtgaatctcagaaaatcccgatcgaccagttcggttgcggtgacaccgctcgtcagtacgttctgatgaacggtaaactgaaagttatcggtttcgacttctctaccaaaatgcagtctatcatccgtgactactctgacctggttatctctcacgctggtaccggttctatcctggactctctgcgtctgaacaaaccgctgatcgtttgcgttaacgactctctgatggacaaccaccagcagcagatcgctgacaaattcgttgaactgggttacgtttggtcttgcgctccgaccgaaaccggtctgatcgctggtctgcgtgcttctcagaccgaaaaactgaaaccgttcccggtttctcacaacccgtctttcgaacgtctgctggttgaaaccatctactcttaa

SEQ ID NO: 2 - protein, 202 aa, Saccharomyces cerevisiae
MGIIEEKALFVTCGATVPFPKLVSCVLSDEFCQELIQYGFVRLIIQFGRNYSSEFEHLVQERGGQRESQKIPIDQFGCGDTARQYVLMNGKLKVIGFDFSTKMQSIIRDYSDLVISHAGTGSILDSLRLNKPLIVCVNDSLMDNHQQQIADKFVELGYVWSCAPTETGLIAGLRASQTEKLKPFPVSHNPSFERLLVETIYS

SEQ ID NO: 3 - DNA, 714 bp, Artificial Sequence
  Description of Artificial Sequence Synthetic polynucleotide
atgaaaaccgcttacctggcttctctggttctgatcgtttctaccgcttacgttatccgtctgatcgctatcctgccgttcttccacacccaggctggtaccgaaaaagacaccaaagacggtgttaacctgctgaaaatccgtaaatcttctaaaaaaccgctgaaaatcttcgttttcctgggttctggtggtcacaccggtgaaatgatccgtctgctggaaaactaccaggacctgctgctgggtaaatctatcgtttacctgggttactctgacgaagcttctcgtcagcgtttcgctcacttcatcaaaaaattcggtcactgcaaagttaaatactacgaattcatgaaagctcgtgaagttaaagctaccctgctgcagtctgttaaaaccatcatcggtaccctggttcagtctttcgttcacgttgttcgtatccgtttcgctatgtgcggttctccgcacctgttcctgctgaacggtccgggtacctgctgcatcatctctttctggctgaaaatcatggaactgctgctgccgctgctgggttcttctcacatcgtttacgttgaatctctggctcgtatcaacaccccgtctctgaccggtaaaatcctgtactgggttgttgacgaattcatcgttcagtggcaggaactgcgtgacaactacctgccgcgttctaaatggttcggtatcctggtttaa

SEQ ID NO: 4 - protein, 237 aa, Saccharomyces cerevisiae
MKTAYLASLVLIVSTAYVIRLIAILPFFHTQAGTEKDTKDGVNLLKIRKSSKKPLKIFVFLGSGGHTGEMIRLLENYQDLLLGKSIVYLGYSDEASRQRFAHFIKKFGHCKVKYYEFMKAREVKATLLQSVKTIIGTLVQSFVHVVRIRFAMCGSPHLFLLNGPGTCCIISFWLKIMELLLPLLGSSHIVYVESLARINTPSLTGKILYWVVDEFIVQWQELRDNYLPRSKWFGILV

SEQ ID NO: 5 - DNA, 1350 bp, Artificial Sequence
  Description of Artificial Sequence Synthetic polynucleotide
atgttcctggaaatcccgcgttggctgctggctctgatcatcctgtacctgtctatcccgctggttgtttactacgttatcccgtacctgttctacggtaacaaatctaccaaaaaacgtatcatcatcttcgttctgggtgacgttggtcactctccgcgtatctgctaccacgctatctctttctctaaactgggttggcaggttgaactgtgcggttacgttgaagacaccctgccgaaaatcatctcttctgacccgaacatcaccgttcaccacatgtctaacctgaaacgtaaaggtggtggtacctctgttatcttcatggttaaaaaagttctgttccaggttctgtctatcttcaaactgctgtgggaactgcgtggttctgactacatcctggttcagaacccgccgtctatcccgatcctgccgatcgctgttctgtacaaactgaccggttgcaaactgatcatcgactggcacaacctggcttactctatcctgcagctgaaattcaaaggtaacttctaccacccgctggttctgatctcttacatggttgaaatgatcttctctaaattcgctgactacaacctgaccgttaccgaagctatgcgtaaatacctgatccagtctttccacctgaacccgaaacgttgcgctgttctgtacgaccgtccggcttctcagttccagccgctggctggtgacatctctcgtcagaaagctctgaccaccaaagctttcatcaaaaactacatccgtgacgacttcgacaccgaaaaaggtgacaaaatcatcgttacctctacctctttcaccccggacgaagacatcggtatcctgctgggtgctctgaaaatctacgaaaactcttacgttaaattcgactcttctctgccgaaaatcctgtgcttcatcaccggtaaaggtccgctgaaagaaaaatacatgaaacaggttgaagaatacgactggaaacgttgccagatcgaattcgtttggctgtctgctgaagactacccgaaactgctgcagctgtgcgactacggtgtttctctgcacacctcttcttctggtctggacctgccgatgaaaatcctggacatgttcggttctggtctgccggttatcgctatgaactacccggttctggacgaactggttcagcacaacgttaacggtctgaaattcgttgaccgtcgtgaactgcacgaatctctgatcttcgctatgaaagacgctgacctgtaccagaaactgaaaaaaaacgttacccaggaagctgaaaaccgttggcagtctaactgggaacgtaccatgcgtgacctgaaactgatccactaa

SEQ ID NO: 6 - protein, 449 aa, Saccharomyces cerevisiae
MFLEIPRWLLALIILYLSIPLVVYYVIPYLFYGNKSTKKRIIIFVLGDVGHSPRICYHAISFSKLGWQVELCGYVEDTLPKIISSDPNITVHHMSNLKRKGGGTSVIFMVKKVLFQVLSIFKLLWELRGSDYILVQNPPSIPILPIAVLYKLTGCKLIIDWHNLAYSILQLKFKGNFYHPLVLISYMVEMIFSKFADYNLTVTEAMRKYLIQSFHLNPKRCAVLYDRPASQFQPLAGDISRQKALTTKAFIKNYIRDDFDTEKGDKIIVTSTSFTPDEDIGILLGALKIYENSYVKFDSSLPKILCFITGKGPLKEKYMKQVEEYDWKRCQIEFVWLSAEDYPKLLQLCDYGVSLHTSSSGLDLPMKILDMFGSGLPVIAMNYPVLDELVQHNVNGLKFVDRRELHESLIFAMKDADLYQKLKKNVTQEAENRWQSNWERTMRDLKLIH

SEQ ID NO: 7 - DNA, 1512 bp, Artificial Sequence
  Description of Artificial Sequence Synthetic polynucleotide
atgatcgaaaaagacaaacgtaccatcgctttcatccacccggacctgggtatcggtggtgctgaacgtctggttgttgacgctgctctgggtctgcagcagcagggtcactctgttatcatctacacctctcactgcgacaaatctcactgcttcgaagaagttaaaaacggtcagctgaaagttgaagtttacggtgacttcctgccgaccaacttcctgggtcgtttcttcatcgttttcgctaccatccgtcagctgtacctggttatccagctgatcctgcagaaaaaagttaacgcttaccagctgatcatcatcgaccagctgtctacctgcatcccgctgctgcacatcttctcttctgctaccctgatgttctactgccacttcccggaccagctgctggctcagcgtgctggtctgctgaaaaaaatctaccgtctgccgttcgacctgatcgaacagttctctgtttctgctgctgacaccgttgttgttaactctaacttcaccaaaaacaccttccaccagaccttcaaatacctgtctaacgacccggacgttatctacccgtgcgttgacctgtctaccatcgaaatcgaagacatcgacaaaaaattcttcaaaaccgttttcaacgaaggtgaccgtttctacctgtctatcaaccgtttcgaaaaaaaaaaagacgttgctctggctatcaaagctttcgctctgtctgaagaccagatcaacgacaacgttaaactggttatctgcggtggttacgacgaacgtgttgctgaaaacgttgaatacctgaaagaactgcagtctctggctgacgaatacgaactgtctcacaccaccatctactaccaggaaatcaaacgtgtttctgacctggaatctttcaaaaccaacaactctaaaatcatcttcctgacctctatctcttcttctctgaaagaactgctgctggaacgtaccgaaatgctgctgtacaccccggcttacgaacacttcggtatcgttccgctggaagctatgaaactgggtaaaccggttctggctgttaacaacggtggtccgctggaaaccatcaaatcttacgttgctggtgaaaacgaatcttctgctaccggttggctgaaaccggctgttccgatccagtgggctaccgctatcgacgaatctcgtaaaatcctgcagaacggttctgttaacttcgaacgtaacggtccgctgcgtgttaaaaaatacttctctcgtgaagctatgacccagtctttcgaagaaaacgttgaaaaagttatctggaaagaaaaaaaatactacccgtgggaaatcttcggtatctctttctctaacttcatcctgcacatggctttcatcaaaatcctgccgaacaacccgtggccgttcctgttcatggctaccttcatggttctgtacttcaaaaactacctgtggggtatctactgggctttcgttttcgctctgtcttacccgtacgaagaaatctaa

SEQ ID NO: 8 - protein, 503 aa, Saccharomyces cerevisiae
MIEKDKRTIAFIHPDLGIGGAERLVVDAALGLQQQGHSVIIYTSHCDKSHCFEEVKNGQLKVEVYGDFLPTNFLGRFFIVFATIRQLYLVIQLILQKKVNAYQLIIIDQLSTCIPLLHIFSSATLMFYCHFPDQLLAQRAGLLKKIYRLPFDLIEQFSVSAADTVVVNSNFTKNTFHQTFKYLSNDPDVIYPCVDLSTIEIEDIDKKFFKTVFNEGDRFYLSINRFEKKKDVALAIKAFALSEDQINDNVKLVICGGYDERVAENVEYLKELQSLADEYELSHTTIYYQEIKRVSDLESFKTNNSKIIFLTSISSSLKELLLERTEMLLYTPAYEHFGIVPLEAMKLGKPVLAVNNGGPLETIKSYVAGENESSATGWLKPAVPIQWATAIDESRKILQNGSVNFERNGPLRVKKYFSREAMTQSFEENVEKVIWKEKKYYPWEIFGISFSNFILHMAFIKILPNNPWPFLFMATFMVLYFKNYLWGIYWAFVFALSYPYEEI

SEQ ID NO: 9 - DNA, 1647 bp, Saccharomyces cerevisiae
atgggcagtgcttggacaaactacaattttgaagaggttaagtctcattttgggttcaaaaaatatgttgtatcatctttagtactagtgtatggactaattaaggttctcacgtggatcttccgtcaatgggtgtattccagcttgaatccgttctccaaaaaatcttcattactgaacagagcagttgcctcctgtggtgagaagaatgtgaaagtttttggtttttttcatccgtattgtaatgctggtggtggtggggaaaaagtgctctggaaagctgtagatatcactttgagaaaagatgctaagaacgttattgtcatttattcaggggattttgtgaatggagagaatgttactccggagaatattctaaataatgtgaaagcgaagttcgattacgacttggattcggatagaatatttttcatttcattgaagctaagatacttggtggattcttcaacatggaagcatttcacgttgattggacaagcaattggatcaatgattctcgcatttgaatccattattcagtgtccacctgatatatggattgatacaatggggtaccctttcagctatcctattattgctaggtttttgaggagaattcctatcgtcacatatacgcattatccgataatgtcaaaagacatgttaaataagctgttcaaaatgcccaagaagggtatcaaagtttacggtaaaatattatactggaaagtttttatgttaatttatcaatccattggttctaaaattgatattgtaatcacaaactcaacatggacaaataaccacataaagcaaatttggcaatccaatacgtgtaaaattatatatcctccatgctctactgagaaattagtagattggaagcaaaagtttggtactgcaaagggtgagagattaaatcaagcaattgtgttggcacaatttcgtcctgagaaacgtcataagttaatcattgagtcctttgcaactttcttgaaaaatttaccggattctgtatcgccaattaaattgataatggcggggtccactagatccaagcaagatgaaaattatgttaaaagtttacaagactggtcagaaaatgtattaaaaattcctaaacatttgatatcattcgaaaaaaatctgcccttcgataagattgaaatattactaaacaaatctactttcggtgttaatgccatgtggaatgagcactttggaattgcagttgtagagtatatggcttccggtttgatccccatagttcatgcctcggcgggcccattgttagatatagttactccatgggatgccaacgggaatatcggaaaagctccaccacaatgggagttacaaaagaaatattttgcaaaactcgaagatgatggtgaaactactggatttttctttaaagagccgagtgatcctgattataacacaaccaaagatcctctgagataccctaatttgtccgaccttttcttacaaattacgaaactggactatgactgcctaagggtgatgggcgcaagaaaccagcagtattcattgtataaattctctgatttgaagtttgataaagattgggaaaactttgtactgaatcctatttgtaaattattagaagaggaggaaaggggctga

SEQ ID NO: 10 - protein, 548 aa, Saccharomyces cerevisiae
MGSAWTNYNFEEVKSHFGFKKYVVSSLVLVYGLIKVLTWIFRQWVYSSLNPFSKKSSLLNRAVASCGEKNVKVFGFFHPYCNAGGGGEKVLWKAVDITLRKDAKNVIVIYSGDFVNGENVTPENILNNVKAKFDYDLDSDRIFFISLKLRYLVDSSTWKHFTLIGQAIGSMILAFESIIQCPPDIWIDTMGYPFSYPIIARFLRRIPIVTYTHYPIMSKDMLNKLFKMPKKGIKVYGKILYWKVFMLIYQSIGSKIDIVITNSTWTNNHIKQIWQSNTCKIIYPPCSTEKLVDWKQKFGTAKGERLNQAIVLAQFRPEKRHKLIIESFATFLKNLPDSVSPIKLIMAGSTRSKQDENYVKSLQDWSENVLKIPKHLISFEKNLPFDKIEILLNKSTFGVNAMWNEHFGIAVVEYMASGLIPIVHASAGPLLDIVTPWDANGNIGKAPPQWELQKKYFAKLEDDGETTGFFFKEPSDPDYNTTKDPLRYPNLSDLFLQITKLDYDCLRVMGARNQQYSLYKFSDLKFDKDWENFVLNPICKLLEEEERG

SEQ ID NO: 11 - DNA, 1113 bp, Escherichia coli
aaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgtatcaccaagtaa

SEQ ID NO: 12 - protein, 370 aa, Escherichia coli [sequence omitted]

SEQ ID NO: 13 - DNA, 330 bp, Bacillus sp.
atgttttgtacattttttgaaaaacatcaccggaagtgggacatactgttagaaaaaagcacgggtgtgatggaagctatgaaagtgacgagtgaggaaaaggaacagctgagcacagcaatcgaccgaatgaatgaaggactggacgcgtttatccagctgtataatgaatcggaaattgatgaaccgcttattcagcttgatgatgatacagccgagttaatgaagcaggcccgagatatgtacggccaggaaaagctaaatgagaaattaaatacaattattaaacagattttatccatctcagtatctgaagaaggagaaaaagaa

SEQ ID NO: 14 - protein, 110 aa, Bacillus sp.
MFCTFFEKHHRKWDILLEKSTGVMEAMKVTSEEKEQLSTAIDRMNEGLDAFIQLYNESEIDEPLIQLDDDTAELMKQARDMYGQEKLNEKLNTIIKQILSISVSEEGEKE

SEQ ID NO: 15 - DNA, 1254 bp, Nicotiana tabacum
gcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcggaattgaagatacttaa

SEQ ID NO: 16 - protein, 417 aa, Nicotiana tabacum
ATQSEYADRLAAAIEAENHCTSQTRLLIDQISLQQGRIVALEEQMKRQDQECRQLRALVQDLESKGIKKLIGNVQMPVAAVVVMACNRADYLEKTIKSILKYQISVASKYPLFISQDGSHPDVRKLALSYDQLTYMQHLDFEPVHTERPGELIAYYKIARHYKWALDQLFYKHNFSRVIILEDDMEIAPDFFDFFEAGATLLDRDKSIMAISSWNDNGQMQFVQDPYALYRSDFFPGLGWMLSKSTWDELSPKWPKAYWDDWLRLKENHRGRQFIRPEVCRTYNFGEHGSSLGQFFKQYLEPIKLNDVQVDWKSMDLSYLLEDNYVKHFGDLVKKAKPIHGADAVLKAFNIDGDVRIQYRDQLDFENIARQFGIFEEWKDGVPRAAYKGIVVFRYQTSRRVFLVGHDSLQQLGIEDT

SEQ ID NO: 17 - DNA, 1344 bp, Homo sapiens
atgcgctttcgtatctataaacgtaaagtgctgatcctgacactggttgttgccgcttgtggttttgttctgtggagcagtaatggtcgtcagcgtaaaaatgaagccctggcacctcctctgctggatgctgaaccggcacgtggtgctggcggtcgtggtggtgatcatccgtctgttgccgttggtattcgtcgtgtgagcaatgtttcggctgcctctctggtcccggctgttcctcaacctgaagctgataacctgaccctgcgctatcgctctctggtgtatcaactgaacttcgatcaaactctgcgtaacgtggataaagcaggcacatgggctcctcgtgaactggtactggtagtccaggtccataatcgtccggaatatctgcgtctgctgctggattctctgcgcaaagctcaaggcatcgataatgtcctggtcatcttctctcatgatttctggagcacggagattaaccagctgattgccggcgtgaatttttgtcctgtgctgcaggtgttttttccgttttctatccaactgtatccgaacgaatttccgggttctgatcctcgtgattgtcctcgtgatctgcctaaaaatgccgctctgaaactgggctgtattaatgccgagtatcctgattcttttggccactatcgtgaggcgaaattttctcagaccaaacatcattggtggtggaaactgcatttcgtgtgggaacgtgtgaaaatcctgcgcgactatgctggcctgattctgtttctggaagaagatcactatctggctccggacttttatcatgtgttcaaaaaaatgtggaaactgaaacagcaggaatgtccagaatgtgatgtgctgtcactgggcacctatagtgcttctcgctccttctatggtatggccgacaaagtggacgttaaaacatggaaatccaccgagcacaacatgggtctggcactgactcgtaatgcctatcaaaaactgattgagtgtaccgacaccttttgtacgtatgatgactataactgggactggaccctgcaatatctgaccgtgagctgtctgccaaaattttggaaagttctggtgcctcagattcctcgtatctttcatgctggcgactgtggtatgcaccataaaaaaacttgccgtccgtcaacacaatctgctcagatcgagtcgctgctgaataataacaaacagtatatgttcccggagactctgacaatttctgaaaaattcaccgtggtcgccatttctccgcctcgtaaaaatggaggttggggcgatatccgtgaccatgaactgtgtaaaagctatcgtcgtctgcagtga

SEQ ID NO: 18 - protein, 447 aa, Homo sapiens
MRFRIYKRKVLILTLVVAACGFVLWSSNGRQRKNEALAPPLLDAEPARGAGGRGGDHPSVAVGIRRVSNVSAASLVPAVPQPEADNLTLRYRSLVYQLNFDQTLRNVDKAGTWAPRELVLVVQVHNRPEYLRLLLDSLRKAQGIDNVLVIFSHDFWSTEINQLIAGVNFCPVLQVFFPFSIQLYPNEFPGSDPRDCPRDLPKNAALKLGCINAEYPDSFGHYREAKFSQTKHHWWWKLHFVWERVKILRDYAGLILFLEEDHYLAPDFYHVFKKMWKLKQQECPECDVLSLGTYSASRSFYGMADKVDVKTWKSTEHNMGLALTRNAYQKLIECTDTFCTYDDYNWDWTLQYLTVSCLPKFWKVLVPQIPRIFHAGDCGMHHKKTCRPSTQSAQIESLLNNNKQYMFPETLTISEKFTVVAISPPRKNGGWGDIRDHELCKSYRRLQ

SEQ ID NO: 19 - DNA, 1329 bp, Bos taurus
ttgaaagaactgacgtccaaaaagagcttgcaagtcccgtccatctactatcacttgccgcacttgctgcaaaacgagggctctttgcaaccggcagttcagatcggcaatggtcgcaccggcgtgagcattgttatgggtatcccgaccgtgaaacgtgaagtgaaaagctatctgattgaaacgctgcatagcctgatcgataacctgtacccggaagaaaaactggactgcgtgattgtcgttttcattggtgaaaccgacacggattatgtgaatggcgttgttgccaatctggaaaaagagttcagcaaagagatcagcagcggcctggttgagatcatttctccgccggagagctattacccggatctgacgaacctgaaagaaaccttcggtgatagcaaagagcgtgtccgttggcgcactaagcagaacctggactattgttttctgatgatgtacgcgcaagaaaagggtacgtattacatccaactggaggacgacattattgtgaagcaaaactacttcaacaccattaagaacttcgcgctgcagctgagcagcgaagagtggatgattctggagttcagccagctgggcttcattggcaagatgtttcaggcaccggacttgaccctgatcgtggagtttatctttatgttctacaaagagaaaccgatcgattggctgctggatcatatcctgtgggtcaaggtctgcaatccggaaaaagatgccaagcattgtgaccgccagaaagcgaatctgcgtattcgttttcgtcctagcctgttccaacacgtgggtctgcacagctctctgaccggtaagatccaaaagctgaccgacaaagattacatgaaaccgctgctgctgaagatccatgtcaacccgccagcagaggtgagcacctcgctgaaagtctaccagggtcacactctggagaaaacctatatgggcgaggacttcttttgggcgattacgcctgttgcgggtgactatatcttgtttaagtttgacaagccggttaatgtagagagctacttgtttcatagcggtaaccaggatcacccaggtgacattctgctgaacaccaccgttgaagtgttgccgctgaaaagcgaaggtctggatatttcgaaagaaacgaaggataagcgtctggaggatggttacttccgtatcggcaagttcgagaatggcgtggctgaaggtatggtcgacccgagcctgaacccgatttccgcatttcgcctgtccgtcatccagaatagcgcggtttgggctatcctgaatgagattcacatcaaaaaggttacgaattaa

SEQ ID NO: 20 - protein, 443 aa, Bos taurus
ILKELTSKKSLQVPSIYYHLPHLLQNEGSLQPAVQIGNGRTGVSIVMGIPTVKREVKSYLIETLHSLIDNLYPEEKLDCVIVVFIGETDTDYVNGVVANLEKEFSKEISSGLVEIISPPESYYPDLTNLKETFGDSKERVRWRTKQNLDYCFLMMYAQEKGTYYIQLEDDIIVKQNYFNTIKNFALQLSSEEWMILEFSQLGFIGKMFQAPDLTLIVEFIFMFYKEKPIDWLLDHILWVKVCNPEKDAKHCDRQKANLRIRFRPSLFQHVGLHSSLTGKIQKLTDKDYMKPLLLKIHVNPPAEVSTSLKVYQGHTLEKTYMGEDFFWAITPVAGDYILFKFDKPVNVESYLFHSGNQDHPGDILLNTTVEVLPLKSEGLDISKETKDKRLEDGYFRIGKFENGVAEGMVDPSLNPISAFRLSVIQNSAVWAILNEIHIKKVTN

SEQ ID NO: 21 - DNA, 822 bp, Helicobacter pylori
atgcgtgtctttattatcagtctgaaccagaaagtgtgtgacaaattcggcctggtgtttcgtgataccacaaccctgctgaataacatcaatgccacccgccacaaagcacagatttttgacgccgtctatagcaaaacgttcgaaggtgggctgcatccactggtgaaaaaacatctgcacccgtatttcattacccagaacatcaaagacatgggcattaccaccaacctgattagcggtgtatccaaattctattatgctctgaaatatcacgccaaattcatgagcctgggcgaactgggctgttatgccagccattatagcctgtgggagaaatgtattgagctgaacgaggccatttgtatcctggaagatgacattacgctgaaagaagatttcaaagagggcctggatttcctggaaaaacacattcaggagctgggctatgttcgtctgatgcatctgctgtatgatgcctccgttaaaagcgaacctctgtcccataaaaaccacgagattcaagagcgtgtcgggatcattaaagcttatagtcacggtgttggcactcagggatatgtgattactccgaaaattgccaaagtgttcaaaaaatgctcccgtaaatgggttgttccggtggatacgatcatggatgccacgtttattcatggggtgaaaaacctggtactgcaaccgtttgtgattgccgatgatgagcaaatttccacgattgtccgtaaagaggagccgtattcccctaaaattgccctgatgcgcgaactgcacttcaaatatctgaaatattggcagtttgtgtga

SEQ ID NO: 22 - protein, 273 aa, Helicobacter pylori
MRVFIISLNQKVCDKFGLVFRDTTTLLNNINATRHKAQIFDAVYSKTFEGGLHPLVKKHLHPYFITQNIKDMGITTNLISGVSKFYYALKYHAKFMSLGELGCYASHYSLWEKCIELNEAICILEDDITLKEDFKEGLDFLEKHIQELGYVRLMHLLYDASVKSEPLSHKNHEIQERVGIIKAYSHGVGTQGYVITPKIAKVFKKCSRKWVVPVDTIMDATFIHGVKNLVLQPFVIADDEQISTIVRKEEPYSPKIALMRELHFKYLKYWQFV

SEQ ID NO: 23 - DNA, 1371 bp, Escherichia coli
atgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 24 - protein, 456 aa, Escherichia coli
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 25 - DNA, 1437 bp, Escherichia coli
atggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaa

SEQ ID NO: 26 - protein, 478 aa, Escherichia coli
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 27 - DNA, 1830 bp, Escherichia coli
atgtgtggaattgttggcgcgatcgcgcaacgtgatgtagcagaaatccttcttgaaggtttacgtcgtctggaataccgcggatatgactctgccggtctggccgttgttgatgcagaaggtcatatgacccgcctgcgtcgcctcggtaaagtccagatgctggcacaggcagcggaagaacatcctctgcatggcggcactggtattgctcacactcgctgggcgacccacggtgaaccttcagaagtgaatgcgcatccgcatgtttctgaacacattgtggtggtgcataacggcatcatcgaaaaccatgaaccgctgcgtgaagagctaaaagcgcgtggctataccttcgtttctgaaaccgacaccgaagtgattgcccatctggtgaactgggagctgaaacaaggcgggactctgcgtgaggccgttctgcgtgctatcccgcagctgcgtggtgcgtacggtacagtgatcatggactcccgtcacccggataccctgctggcggcacgttctggtagtccgctggtgattggcctggggatgggcgaaaactttatcgcttctgaccagctggcgctgttgccggtgacccgtcgctttatcttccttgaagagggcgatattgcggaaatcactcgccgttcggtaaacatcttcgataaaactggcgcggaagtaaaacgtcaggatatcgaatccaatctgcaatatgacgcgggcgataaaggcatttaccgtcactacatgcagaaagagatctacgaacagccgaacgcgatcaaaaacacccttaccggacgcatcagccacggtcaggttgatttaagcgagctgggaccgaacgccgacgaactgctgtcgaaggttgagcatattcagatcctcgcctgtggtacttcttataactccggtatggtttcccgctactggtttgaatcgctagcaggtattccgtgcgacgtcgaaatcgcctctgaattccgctatcgcaaatctgccgtgcgtcgtaacagcctgatgatcaccttgtcacagtctggcgaaaccgcggataccctggctggcctgcgtctgtcgaaagagctgggttaccttggttcactggcaatctgtaacgttccgggttcttctctggtgcgcgaatccgatctggcgctaatgaccaacgcgggtacagaaatcggcgtggcatccactaaagcattcaccactcagttaactgtgctgttgatgctggtggcgaagctgtctcgcctgaaaggtctggatgcctccattgaacatgacatcgtgcatggtctgcaggcgctgccgagccgtattgagcagatgctgtctcaggacaaacgcattgaagcgctggcagaagatttctctgacaaacatcacgcgctgttcctgggccgtggcgatcagtacccaatcgcgctggaaggcgcattgaagttgaaagagatctcttacattcacgctgaagcctacgctgctggcgaactgaaacacggtccgctggcgctaattgatgccgatatgccggttattgttgttgcaccgaacaacgaattgctggaaaaactgaaatccaacattgaagaagttcgcgcgcgtggcggtcagttgtatgtcttcgccgatcaggatgcgggttttgtaagtagcgataacatgcacatcatcgagatgccgcatgtggaagaggtgattgcaccgatcttctacaccgttccgctgcagctgctggcttaccatgtcgcgctgatcaaaggcaccgacgttgaccagccgcgtaacctggcaaaatcggttacggttgagtaa

SEQ ID NO: 28 - protein, 609 aa, Escherichia coli
MCGIVGAIAQRDVAEILLEGLRRLEYRGYDSAGLAVVDAEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYRHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLGRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 29 - DNA, 2028 bp, Photobacterium damselae
atgaaaaaaatcctgaccgtgctgtccatctttatcctgtctgcctgtaatagcgacaataccagcctgaaagagactgttagcagcaattcagcggatgttgtggaaaccgaaacttatcaactgacgccgatcgatgctccttcttcgttcctgagccattcttgggaacagacctgtggtacaccaattctgaacgagtccgacaaacaggccatttccttcgattttgttgccccggaactgaaacaagacgagaaatattgcttcaccttcaaaggcattaccggtgatcatcgttatatcacgaacaccactctgactgtcgtagcaccgacactggaagtgtatatcgaccatgccagcctgcctagtctgcagcaactgatccatattatccaggcgaaagacgaatatccgagcaaccagcgttttgtgagctggaaacgtgttactgtggatgccgacaacgccaataaactgaacattcacacctatcctctgaaaggcaataacaccagccctgagatggtagcggcgattgatgagtatgcccagagcaaaaaccgtctgaacattgagttctataccaatacggcccacgtgtttaataacctgccgccaatcattcaacctctgtataacaacgagaaagtgaaaatcagccacatttcgctgtatgatgatggcagtagcgagtatgttagcctgtatcagtggaaagacaccccgaataaaatcgagactctggagggtgaagtttctctgctggccaactatctggccggtacaagtcctgatgctccgaaagggatgggtaaccgctataattggcacaaactgtatgacaccgactattattttctgcgcgaggattatctggacgtggaagccaatctgcatgatctgcgcgattatctgggttctagcgccaaacaaatgccgtgggatgaatttgctaaactgtccgattctcagcaaaccctgttcctggacatcgttggctttgataaagagcagctgcaacagcagtatagccagtcaccgctgccgaacttcatttttactggcaccaccacatgggcagggggtgagacaaaagagtattatgctcaacaacaggtgaacgtcatcaacaatgccattaacgaaacctccccatattatctgggtaaagactatgacctgttctttaaaggccatccggctggaggagtgattaatgatattatcctgggctcctttcctgacatgattaacattccggcgaaaatctcatttgaggtgctgatgatgactgatatgctgccggataccgttgctggaattgcctcttccctgtatttcaccattcctgccgacaaagtgaacttcatcgtgttcaccagcagtgataccattacagaccgtgaagaagcgctgaaatctcctctggttcaggtgatgctgacactgggtatcgtgaaagaaaaagacgtcctgttttgggccgaccataaagtgaatagcatggaggtggccatcgacgaagcgtgtactcgtattatcgccaaacgtcagcctaccgcttcagatctgcgtctggttatcgccattatcaaaacgatcaccgatctggagcgtattggagatgttgccgaaagcattgccaaagttgccctggagagcttttctaacaaacagtataatctgctggtcagcctggaatctctgggtcaacacaccgttcgtatgctgcatgaagtgctggatgcttttgcccgtatggatgtgaaagcagccattgaagtctatcaggaggatgaccgtatcgatcaggaatatgagagcattgtccgtcaactgatggcccatatgatggaagatccgtctagcattccgaatgtgatgaaagtgatgtgggcagctcgtagtattgaacgtgtgggtgaccgctgccagaacatttgtgagtatatcatctatttcgtaaaaggcaaagatgttcgccacaccaaaccggatgacttcggtactatgctggactga

SEQ ID NO: 30 - protein, 675 aa, Photobacterium damselae
MKKILTVLSIFILSACNSDNTSLKETVSSNSADVVETETYQLTPIDAPSSFLSHSWEQTCGTPILNESDKQAISFDFVAPELKQDEKYCFTFKGITGDHRYITNTTLTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWADHKVNSMEVAIDEACTRIIAKRQPTASDLRLVIAIIKTITDLERIGDVAESIAKVALESFSNKQYNLLVSLESLGQHTVRMLHEVLDAFARMDVKAAIEVYQEDDRIDQEYESIVRQLMAHMMEDPSSIPNVMKVMWAARSIERVGDRCQNICEYIIYFVKGKDVRHTKPDDFGTMLD

SEQ ID NO: 31 - DNA, 2367 bp, Artificial Sequence
  Description of Artificial Sequence Synthetic polynucleotide
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgtatcaccaaggcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcggaattgaagatacttaa

SEQ ID NO: 32 - DNA, 2370 bp, Artificial Sequence
  Description of Artificial Sequence Synthetic polynucleotide
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgtatcaccaagcgtcagcgtaaaaatgaagccctggcacctcctctgctggatgctgaaccggcacgtggtgctggcggtcgtggtggtgatcatccgtctgttgccgttggtattcgtcgtgtgagcaatgtttcggctgcctctctggtcccggctgttcctcaacctgaagctgataacctgaccctgcgctatcgctctctggtgtatcaactgaacttcgatcaaactctgcgtaacgtggataaagcaggcacatgggctcctcgtgaactggtactggtagtccaggtccataatcgtccggaatatctgcgtctgctgctggattctctgcgcaaagctcaaggcatcgataatgtcctggtcatcttctctcatgatttctggagcacggagattaaccagctgattgccggcgtgaatttttgtcctgtgctgcaggtgttttttccgttttctatccaactgtatccgaacgaatttccgggttctgatcctcgtgattgtcctcgtgatctgcctaaaaatgccgctctgaaactgggctgtattaatgccgagtatcctgattcttttggccactatcgtgaggcgaaattttctcagaccaaacatcattggtggtggaaactgcatttcgtgtgggaacgtgtgaaaatcctgcgcgactatgctggcctgattctgtttctggaagaagatcactatctggctccggacttttatcatgtgttcaaaaaaatgtggaaactgaaacagcaggaatgtccagaatgtgatgtgctgtcactgggcacctatagtgcttctcgctccttctatggtatggccgacaaagtggacgttaaaacatggaaatccaccgagcacaacatgggtctggcactgactcgtaatgcctatcaaaaactgattgagtgtaccgacaccttttgtacgtatgatgactataactgggactggaccctgcaatatctgaccgtgagctgtctgccaaaattttggaaagttctggtgcctcagattcctcgtatctttcatgctggcgactgtggtatgcaccataaaaaaacttgccgtccgtcaacacaatctgctcagatcgagtcgctgctgaataataacaaacagtatatgttcccggagactctgacaatttctgaaaaattcaccgtggtcgccatttctccgcctcgtaaaaatggaggttggggcgatatccgtgaccatgaactgtgtaaaagctatcgtcgtctgcagtga

SEQ ID NO: 33 - DNA, 2445 bp, Artificial Sequence
  Description of Artificial Sequence Synthetic polynucleotide
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgtatcaccaagattttgaaagaactgacgtccaaaaagagcttgcaagtcccgtccatctactatcacttgccgcacttgctgcaaaacgagggctctttgcaaccggcagttcagatcggcaatggtcgcaccggcgtgagcattgttatgggtatcccgaccgtgaaacgtgaagtgaaaagctatctgattgaaacgctgcatagcctgatcgataacctgtacccggaagaaaaactggactgcgtgattgtcgttttcattggtgaaaccgacacggattatgtgaatggcgttgttgccaatctggaaaaagagttcagcaaagagatcagcagcggcctggttgagatcatttctccgccggagagctattacccggatctgacgaacctgaaagaaaccttcggtgatagcaaagagcgtgtccgttggcgcactaagcagaacctggactattgttttctgatgatgtacgcgcaagaaaagggtacgtattacatccaactggaggacgacattattgtgaagcaaaactacttcaacaccattaagaacttcgcgctgcagctgagcagcgaagagtggatgattctggagttcagccagctgggcttcattggcaagatgtttcaggcaccggacttgaccctgatcgtggagtttatctttatgttctacaaagagaaaccgatcgattggctgctggatcatatcctgtgggtcaaggtctgcaatccggaaaaagatgccaagcattgtgaccgccagaaagcgaatctgcgtattcgttttcgtcctagcctgttccaacacgtgggtctgcacagctctctgaccggtaagatccaaaagctgaccgacaaagattacatgaaaccgctgctgctgaagatccatgtcaacccgccagcagaggtgagcacctcgctgaaagtctaccagggtcacactctggagaaaacctatatgggcgaggacttcttttgggcgattacgcctgttgcgggtgactatatcttgtttaagtttgacaagccggttaatgtagagagctacttgtttcatagcggtaaccaggatcacccaggtgacattctgctgaacaccaccgttgaagtgttgccgctgaaaagcgaaggtctggatatttcgaaagaaacgaaggataagcgtctggaggatggttacttccgtatcggcaagttcgagaatggcgtggctgaaggtatggtcgacccgagcctgaacccgatttccgcatttcgcctgtccgtcatccagaatagcgcggtttgggctatcctgaatgagattcacatcaaaaaggttacgaattaa

SEQ ID NO: 34 - DNA, 2247 bp, Artificial Sequence
  Description of Artificial Sequence Synthetic polynucleotide
atgaaattgttctacaaaccgggtgcctgctctctcgcttcccatatcaccctgcgtgagagcggaaaggattttaccctcgtcagtgtggatttaatgaaaaaacgtctcgaaaacggtgacgattactttgccgttaaccctaaggggcaggtgcctgcattgctgctggatgacggtactttgctgacggaaggcgtagcgattatgcagtatcttgccgacagcgtccccgaccgccagttgctggcaccggtaaacagtatttcccgctataaaaccatcgaatggctgaattacatcgccaccgagctgcataaaggtttcacacctctgtttcgccctgatacaccggaagagtacaaaccgacagttcgcgcgcagctggagaagaagctgcaatatgtgaacgaggcactgaaggatgagcactggatctgcgggcaaagatttacaattgctgatgcctatctgtttacggttctgcgctgggcatacgcggtgaaactgaatctggaagggttagagcacattgcagcatttatgcaacgtatggctgaacgtccggaagtacaagacgcgctgtcagcggaaggcttaaagggcagtgcttggacaaactacaattttgaagaggttaagtctcattttgggttcaaaaaatatgttgtatcatctttagtactagtgtatggactaattaaggttctcacgtggatcttccgtcaatgggtgtattccagcttgaatccgttctccaaaaaatcttcattactgaacagagcagttgcctcctgtggtgagaagaatgtgaaagtttttggtttttttcatccgtattgtaatgctggtggtggtggggaaaaagtgctctggaaagctgtagatatcactttgagaaaagatgctaagaacgttattgtcatttattcaggggattttgtgaatggagagaatgttactccggagaatattctaaataatgtgaaagcgaagttcgattacgacttggattcggatagaatatttttcatttcattgaagctaagatacttggtggattcttcaacatggaagcatttcacgttgattggacaagcaattggatcaatgattctcgcatttgaatccattattcagtgtccacctgatatatggattgatacaatggggtaccctttcagctatcctattattgctaggtttttgaggagaattcctatcgtcacatatacgcattatccgataatgtcaaaagacatgttaaataagctgttcaaaatgcccaagaagggtatcaaagtttacggtaaaatattatactggaaagtttttatgttaatttatcaatccattggttctaaaattgatattgtaatcacaaactcaacatggacaaataaccacataaagcaaatttggcaatccaatacgtgtaaaattatatatcctccatgctctactgagaaattagtagattggaagcaaaagtttggtactgcaaagggtgagagattaaatcaagcaattgtgttggcacaatttcgtcctgagaaacgtcataagttaatcattgagtcctttgcaactttcttgaaaaatttaccggattctgtatcgccaattaaattgataatggcggggtccactagatccaagcaagatgaaaattatgttaaaagtttacaagactggtcagaaaatgtattaaaaattcctaaacatttgatatcattcgaaaaaaatctgcccttcgataagattgaaatattactaaacaaatctactttcggtgttaatgccatgtggaatgagcactttggaattgcagttgtagagtatatggcttccggtttgatccccatagttcatgcctcggcgggcccattgttagatatagttactccatgggatgccaacgggaatatcggaaaagctccaccacaatgggagttacaaaagaaatattttgcaaaactcgaagatgatggtgaaactactggatttttctttaaagagccgagtgatcctgattataacacaaccaaagatcctctgagataccctaatttgtccgaccttttcttacaaattacgaaactggactatgactgcctaagggtgatgggcgcaagaaaccagcagtattcattgtataaattctctgatttgaagtttgataaagattgggaaaactttgtactgaatcctatttgtaaattattagaagaggaggaaaggggctga

SEQ ID NO: 35 - protein, 6 aa, Artificial Sequence
  Description of Artificial Sequence Synthetic 6xHis tag
HHHHHH

SEQ ID NO: 36 - protein, 5 aa, Artificial Sequence
  Description of Artificial Sequence Synthetic peptide
DQNAT